(19) United States
(12) Patent Application Publication
(10) Pub. No.: US 2023/0348878 A1
(43) Pub. Date: Nov. 2, 2023

(54) ENHANCEMENT OF SAFETY AND PRECISION FOR CRISPR-Cas INDUCED GENE EDITING BY VARIANTS OF DNA POLYMERASE USING CAS-PLUS VARIANTS

(71) Applicant: New York University, New York, NY (US)

(72) Inventors: Chengzu Long, New York, NY (US); Qiaoyan Yang, New York, NY (US)

(21) Appl. No.: 18/308,530

(22) Filed: Apr. 27, 2023

Related U.S. Application Data
(60) Provisional application No. 63/433,353, filed on Dec. 16, 2022.
(60) Provisional application No. 63/335,625, filed on Apr. 27, 2022.

(51) Int. Cl. C12N 9/22; C07K 14/005; C12N 15/10

(52) U.S. Cl. CPC C12N 9/22; C07K 14/005; C12N 15/102; C12N 2310/20

(57) ABSTRACT

Provided are compositions and methods that include an engineered DNA polymerase used in combination with a Cas9 protein. The combination exhibits improved on-target chromosomal alterations, increases the proportion of precise 1- to 3-base-pair insertions at target sites, and reduces translocations caused by previously available systems.

### CROSS-REFERENCE TO RELATED APPLICATIONS

This application claims priority to U.S. provisional patent application No. 63/335,625, filed on Apr. 27, 2022, and to U.S. provisional patent application No. 63/433,353, filed on Dec. 16, 2022, the entire disclosures of each of which are incorporated herein by reference.

### SEQUENCE LISTING

The instant application contains a sequence listing which has been submitted in .xml format and is hereby incorporated by reference in its entirety. Said .xml file is named “058636_00597_ST26.xml”, was created on Apr. 26, 2023, and is 107,494 bytes in size.

### RELATED INFORMATION

The engineered CRISPR/Cas9 system is a powerful tool for sequence-specific gene editing⁽¹⁻⁴⁾. However, it can also generate undesired large deletions^{(5, 6)}, chromosomal translocations⁽⁷⁾, chromothripsis⁽⁸⁾, and other complex chromosome rearrangements as well as off-target effect. Although numerous strategies have been developed to minimize CRISPR/Cas9-mediated off-target effects⁽⁹⁾, few approaches can mitigate collateral on-target DNA damage. Cas9 cleaves target DNA to produce either blunt ends or staggered ends with 5′) overhangs⁽¹⁰⁾. Repair of these ends typically occurs through canonical non-homologous end joining (c-NHEJ) or microhomology-mediated end joining (MMEJ)⁽¹¹⁾. The choice of repair pathway determines CRISPR/Cas9 editing outcomes. MMEJ repair often results in deletions, particularly large deletions^{(12, 13)}. Systematic analyses of Cas9 target sites have revealed that insertions arising from the c-NHEJ pathway are precise and predictable⁽¹⁴⁻¹⁶⁾. The frequency and pattern of insertions depend highly on the local sequence surrounding the Cas9 cut site⁽¹⁷⁾. But methods that can enhance these outcomes are limited. Hence there remains an ongoing need for improved safety and precision of Cas-enzyme based DNA editing. The present disclosure is pertinent to this need.

### BRIEF SUMMARY

The present disclosure provides compositions and methods for precise genome editing. The compositions include DNA polymerases, representative examples of which are described further below. In embodiments, the disclosure provides a fusion protein comprising a DNA polymerase segment, which may comprise changes in amino acid sequence relative to a reference DNA polymerase sequence (i.e., a wild type DNA polymerase sequence), representative amino acid changes being described further herein, and a segment of an MS2 bacteriophage coat protein. The DNA polymerase alone or a described fusion protein operates with a Cas and one or more guide RNAs to produce one or more indels. The Cas may also comprise changes in amino acid sequences relative to a reference sequence (i.e., a wild type Cas sequence), representative amino acid changes being described further herein.

In embodiments, the indel is produced using non-homologous end joining (NHEJ), which is at least in part facilitated by the described DNA polymerase that is a component of a genome editing system encompassed by the disclosure. The disclosure provides for producing an indel in a DNA repair template free manner. The described protein(s) functions as a component of a CRISPR system in the nucleus of the cell. Accordingly, any protein described herein may include at least one nuclear localization signal. Where a described fusion protein is used it may also include one or more linkers that separate, for example, the DNA polymerase and the MS2, and/or that separate a segment of the fusion protein from the nuclear localization signal. In embodiments, a fusion protein comprises a self-cleaving peptide sequence, which can, for example, promote ribosomal skipping during translation. Thus, the fusion protein may be encoded by an mRNA that encodes additional amino acids on the N- or C-terminal ends of the fusion protein which, by operation of a self-cleaving peptide sequence, are not translated as a part of a contiguous polypeptide that comprises the DNA polymerase and the MS2 protein segment.

In an aspect, the disclosure comprises a complex comprising a Cas enzyme, a guide RNA optionally comprising MS2 bacteriophage coat protein binding sites, a protein comprising a DNA polymerase, and optionally also comprising an MS2 binding protein. In non-limiting embodiments the guide RNA comprises comprise MS2 protein binding sequences when the DNA polymerase is used with an MS2 protein component. Cells comprising a described DNA polymerase or fusion protein comprising the DNA polymerase and a guide RNA are also included. Pharmaceutical compositions comprising the described proteins are also provided. Such compositions may also comprise a guide RNA and a Cas enzyme. Cells comprising the described proteins and complexes are also included. The disclosure also provides expression vectors and cDNAs encoding the described proteins, as well as kits comprising the same and/or additional components.

In embodiments, the disclosure provides for reducing translocation events. For example, in situations where more than one chromosomal location is targeted by a Cas9 or other site-specific nuclease (other than a described CasPlus system), concurrent cleavage at more than one location on one or more chromosomes creates a demonstrated risk of translocation events. The present disclosure demonstrates that such translocation events can be reduced by using a described CasPlus system. Thus, the CasPlus system can be used, for example, to disrupt one or more genes with different targeting guide RNAs and creating indels at more than one location, while reducing the likelihood of a translocation relative to other DNA editing enzymes. In embodiments, a reduction in translocation events as compared to previous approaches is achieved in any eukaryotic cell type, including but not limited to lymphocytes and leukocytes, such as T cells, including but not necessarily limited to a chimeric antigen receptor (CAR) expressing T cell or other type of genetically modified T cell that may be modified using any other guide directed nuclease.

In another aspect, the disclosure provides a method for producing an indel at a selected chromosome locus in a cell. The method comprises introducing into the cell a described protein, a Cas enzyme, and a guide RNA optionally comprising MS2 protein binding sites, wherein the guide RNA directs the Cas enzyme, the DNA polymerase and optionally the MS2 binding protein to the selected chromosome locus, to thereby produce the indel. In embodiments, the indel corrects a mutation in an open reading frame encoded by the selected chromosome locus or converts a sequence into an open reading frame. In embodiments, the selected chromosome locus comprises a mutation in a gene that is correlated with a monogenic disease. In one non-limiting embodiment, the monogenic disease is muscular dystrophy, and wherein the selected chromosome locus includes a gene that includes a mutated dystrophin protein. In this regard, Duchenne muscular dystrophy (DMD) is a debilitating neuromuscular disorder leading to degeneration of cardiac and skeletal muscles⁽¹⁸⁾ and results from inactivating mutations in the X-linked dystrophin gene (DMD)⁽¹⁹⁾. Dilated cardiomyopathy (DCM) is a common and lethal feature of DMD⁽²⁰⁾ that lacks curative treatment. We have previously used CRISPR-Cas9 to rectify DMD mutations in cultured human cells and mdx mice⁽²¹⁻²³⁾; however, undesired DNA damage at edited DMD sites, a safety concern in human therapy, were not evaluated. Thus, in an embodiment, the indel corrects the gene encoding the mutated dystrophin protein with, for example, a lower frequency of off-target modifications, relative to previous approaches. In certain examples, the indel comprises a one or two base pair insertion. In embodiments, the monogenic disease cystic fibrosis, and wherein the selected chromosome locus includes a gene that includes a mutated protein gene that is correlated with cystic fibrosis. In one embodiment, the described system corrects a F508del in the gene that encodes cystic fibrosis transmembrane conductance regulator (CFTR) protein.

### BRIEF DESCRIPTION OF THE FIGURES

FIGS. 1A-1D. Identification of T4 and RB69 DNA polymerase as proteins that favor CasPlus editing. FIG. 1A. A schematic showing two functions of the wild-type T4 DNA polymerase-mediated CasPlus system in cells: enhancing 1-bp insertions via promoting staggered end fill-in (top DNA repair pathway) and inhibiting MMEJ-dependent deletions via disrupting the annealing of MHs (bottom DNA repair pathway). FIG. 1B. A workflow showing the DNA polymerase selection process in tdTomato reporter cells. Briefly, vectors that either expressed Cas9, GFP or tdTomato-sgRNA alone, or in combination with a distinct DNA polymerase, are transfected into tdTomato reporter cells. Transfected cells are sorted into populations expressing either only GFP (tdTomato⁻/GFP⁺) or both tdTomato and GFP (tdTomato⁺/GFP⁺), for DNA isolation and high-throughput sequencing. FIG. 1C. Frequency of Cas9-induced indels upon the overexpression of only Cas9 (termed CTR), or in combination with T4, RB69 and T7 DNA polymerase in tdTomato reporter cells. The tdTomato⁺/GFP⁺ and tdTomato⁻/GFP⁺ cells are sorted as described above. The upper and lower dashed lines show the frequency of deletions and 2-bp insertions, respectively, in cells with Cas9 only treatment (CTR). FIG. 1D. Template-dependent insertion of one or two base-pairs among all treatment groups. Templated 1-bp insertions indicate that the inserted one nucleotide is identical to the nucleotide at position −4 and templated 2-bp insertions indicate that the inserted two nucleotides are identical to the nucleotides at position −5 and −4, if counting the NGG PAM sequences as position 0-2. FIG. 1E. Western blot assay performed in tdTomato reporter cells overexpressing T4, RB69 and T7 DNA polymerase. The arrows point to the correct size bands for each DNA polymerase

FIGS. 2A-2H. T4 DNA polymerase mutant D219A (T4-D219A) improves T4 DNA polymerase-mediated CasPlus editing efficiency. FIG. 2A. A schematic showing that engineered T4 DNA polymerase mutants can promote the fill-in process and 1-bp insertions at Cas9-induced DSB ends with 1-bp overhangs. FIG. 2B. A schematic showing the location of all T4 DNA polymerase mutants tested and the corresponding DNA mutation frequency induced by the mutation(s) relative to T4-WT DNA polymerase. The mutation frequency was calculated according to published literatures (24-26). FIG. 2C. Frequency of Cas9-induced indels at TS11 in CTR or Cas9 and T4 DNA polymerase mutants co-overexpressed cells. The sequence of TS11 is shown in Table 1. The upper and lower dashed lines show the frequency of deletions and 1-bp insertions, respectively, in cells with Cas9-WT and T4-WT overexpression. The arrowheads point to the columns representing 1-bp insertions (left) and deletions (right) in cells with Cas9-WT and T4-D219A overexpression. FIGS. 2D-F. Frequency of Cas9-induced indels at TS2, TS10 and TS12 (FIG. 2D), TS17 and TS18 (FIG. 2E) or TS26 (FIG. 2F) in CTR, T4-WT or T4-D219A overexpressed cells. The T4-D219A mutant improves the insertions frequency at the expense of deletions across all genomic sites shown, relative to T4-WT. The target site sequences are shown in Table 1. FIG. 2G. A schematic demonstrating the capacity of T4 DNA polymerase to fill-in the 5-8 bp overhangs generated by Cas12a. FIG. 2H. Frequency of Cas12a-induced insertions and deletions in cells transfected with Cas12a alone or co-transfected with Cas12a and T4-WT or T4-D219A. The sequences of the guide RNA Lb1 is shown in Table 1.

FIGS. 3A-3B. RB69 DNA polymerase mutant D222A (RB69-D222A) improves RB69 DNA polymerase-mediated CasPlus editing efficiency. FIG. 3A. Frequency of Cas9-induced indels in tdTomato⁺/GFP⁺ cells and tdTomato⁻/GPF⁺ cells sorted from tdTomato reporter cells that were co-transfected with Cas9-WT and either RB69-WT or RB69-D222A. FIG. 3B. Frequency of Cas9-induced indels at TS2, TS11 and TS12 in cells co-transfected with Cas9-WT and either RB69-WT or RB69-D222A. The RB69-D222A mutant improves the frequency of insertions across these genomic sites.

FIGS. 4A-4F. Combination of Cas9 variants and T4 DNA polymerase enhances 1-bp insertions at Cas9 target sites that predominantly produce deletions with Cas9-WT and T4-WT. FIG. 4A. Schematics showing at the sites, where Cas9-WT induces blunt end DSBs, producing deletions, some engineered Cas9 variants can facilitate the generation of 1-bp overhangs at these sites, therefore the addition of T4 DNA polymerase can generate 1-bp insertions. FIG. 4B. A schematic demonstrating the mutation sites of the Cas9 variants tested. All the mutations are within the link II (L-II) region. FIG. 4C. Frequency of Cas9-induced indels at TS11 in cells transfected with Cas9-WT or Cas9 variants. The upper and lower dashed lines show the frequency of deletions and 1-bp insertions, respectively, in cells with Cas9-WT overexpression. The arrowheads point to the columns that represent 1-bp insertions or deletions in cells with overexpression of Cas9 variants F916P, F916del, F919P or Q920P. FIG. 4D. Frequency of Cas9-induced indels at TS11 in cells co-transfected with T4-WT and either Cas9-WT or Cas9 variants. FIG. 4E-FIG. 4F. Frequency of Cas9-induced indels at TS19 or TS22 (E), TS24, TS25 and TS26 (F) in cells transfected with Cas9-WT, Cas9 variants F916P or F916del alone, or in combination with either T4-WT or T4-D219A. The arrowheads point to the columns that represent 1-bp insertions and deletions in cells that exhibit an increase in 1-bp insertions at the expense of deletions, in comparison to cells with only Cas9-WT overexpression.

FIGS. 5A-5E. Combination of Cas9 variants and T4 DNA polymerase enhances the production of longer insertions (2 to 4 bps). FIG. 5A. Schematics showing at the sites where Cas9-WT produces DSB ends with 1-bp overhangs, leading to the production of edits with 1-bp insertions, engineered Cas9 variants can facilitate the generation of 2-bp overhangs at these sites, thereby generating 2-bp insertions in the presence of T4 DNA polymerase. FIG. 5B. Frequency of Cas9-induced indels for GFP⁺ populations isolated from tdTomato reporter cells transfected with Cas9 or Cas9 variants. FIG. 5C. Frequency of Cas9-induced indels for GFP⁺ populations isolated from tdTomato reporter cells co-transfected with T4-WT and either Cas9-WT or Cas9 variants. The arrowheads point to the column representing 3-bp insertions. FIG. 5D. Frequency of Cas9-induced indels at TS5, TS17 and TS18 in cells transfected with Cas9-WT, Cas9 variant F916P or Cas9 variant F916del alone, or in conjunction with either T4-WT or T4-D219A. The arrowheads point to the columns representing the significant increase in longer insertions in cells co-transfection with T4 DNA polymerase and Cas9 variants F916P or F916del in comparison to that in cells co-transfected with T4-WT and Cas9-WT. FIG. 5E. Designs of different version of T4 DNA polymerase-mediated CasPlus system. CasPlus-V1 is the combination of Cas9-WT and T4-WT. CasPlus-V2 labels the combination of Cas9-WT and T4-D219A. CasPlus-V3 and V4 use the combination of Cas9 variants and either T4-WT or T4-D219A, respectively. CasPlus-V3 and V4 are further divided into subcategories based on the Cas9 variant that is used. Cas9 variants F916P, F916del, R920P and Q920P are named V3.1, V3.2. V3.3 and V3.4, respectively, in CasPlus-V3; or V4.1, V4.2, V4.3 and V4.4, respectively, in CasPlus-V4. All T4 DNA polymerases are MS2-targeted.

FIGS. 6A-6G. CasPlus system efficiently represses large deletions. FIG. 6A. Schematics showing that CasPlus represses large deletions via inhibiting long-range end resection. FIG. 6B. Schematics showing the locations of the primers sets used for amplifying the distal or proximal region of TS10. FIG. 6C. Induced pluripotent stem cells (iPSCs) with DMD exon 52 deletion are transfected with Cas9, CasPlus-V1 or CasPlus-V2 to target DMD exon 51. GFP⁺ cells are sorted and isolated for PCR amplification. The PCR gel image is shown on the left whereas the Sanger sequencing result for the lower bands is shown on the right. The sequence in FIG. 6C is 5′-GGTGGGTGACCTGGGAATTGATTATT-3′ (SEQ ID NO: 1). FIG. 6D. Schematics showing the locations of the primers sets used for amplifying the distal or proximal region of TS9. FIG. 6E. Induced pluripotent stem cells (iPSCs) with DMD exon 52 deletion are transfected with Cas9, CasPlus-V1 or CasPlus-V2 to target DMD exon 53. GFP⁺ cells are sorted and isolated for PCR amplification. The PCR gel image is shown on the left whereas the Sanger sequencing result for the lower bands is shown on the right. FIGS. 6F-6G. Depth of PacBio reads at DMD exon 51 (FIG. 6F) or 53 (FIG. 6G) in untreated, Cas9-, CasPlus-V1-, CasPlus-V2-edited iPSCs with DMD exon 52 deletion. The sequence in FIG. 6C is: 5′-GGTGGGTGACCTGGGAATTGATTATT-3′(SEQ ID NO: 1). The sequence in FIG. 6E is: 5′-TATTTTAATATTTGTCAGTGGGATGA-3′(SEQ ID NO: 2).

FIGS. 7A-7F. Enhanced correction of DMD exon 52 deletion in iPSCs via CasPlus editing. FIG. 7A. DMD deletion of exon 52 results in generating a premature stop codon in exon 53 which disrupts dystrophin expression. Two strategies are available for the restoration of dystrophin expression via 1-bp insertions by CasPlus editing. FIG. 7B. All the available guide RNAs that contain a NGG as the PAM sequences are shown on DMD 3′ end of exon 51 (TS 10 and TS27) and 5′ end of exon 53 (TS9, TS28, TS29, TS30 and TS31). FIG. 7C. The frequency of 1-bp insertions, other reframed indels (3n+1, n≠0) or other indels (3n and 3n+2) induced by Cas9 in iPSCs transfected with Cas9, CasPlus-V1 or CasPlus-V2. FIG. 7D. The frequency of mRNA alleles with 1-bp insertions, other reframed indels or other indels in cardiomyocytes differentiated from iPSCs transfected with Cas9, CasPlus-V1 or CasPlus-V2. SC. Single clone with 1-bp insertion selected from TS10 or TS9 edited cell pool was here as positive control. FIG. 7E. RT-PCR analysis on cardiomyocytes differentiated from iPSCs transfected with Cas9, CasPlus-V1 or CasPlus-V2. Cells transfected with Cas9 induced whole exon 51 or exon 53 skipping (lower bands with arrows). The Sanger sequencing results of the lower bands are shown on the right. FIG. 7F. Western blot analysis on cardiomyocytes differentiated from iPSCs transfected with Cas9, CasPlus-V1 or CasPlus-V2. The sequences in FIG. 7B for Exon 51 are: Top: 5′-TGACCTTGAGGATATCAACGAGATGATCATCAAGCAGAAGGTATGA-3′ (SEQ ID NO: 3); Bot: 5′-TCATACCTTCTGCTTGATGATCATCTCGTTGATATCCTCAAGGTCA-3′ (SEQ ID NO: 4). For Exon 53 the sequences are: Top: 5′-aGTTGAAAGAATTCAGAATCAGTGGGATGAAGTACAAGAACACCTTCAGAACCG GAGGCAACAGTT; and GA-3′ (SEQ ID NO: 5) and Bot: 5′-TCAACTGTTGCCTCCGGTTCTGAAGGTGTTCTTGTACTTCATCCCACTGATTCTGA ATTCTTTCAACT-3′ (SEQ ID NO: 6). The sequence for in FIG. 7E for Exon 50-Exon is: 5′-CACTATTGGAGCCTTTGAAAGAATTCAG-3′ (SEQ ID NO: 7); The sequence in FIG. 7E for Exon 51-Exon 54: 5′-TCATCAAGCAGAAGCAGTTGGCCAAAGA-3′ (SEQ ID NO: 8).

FIGS. 8A-8J. Exogenous template-independent correction of CFTR F508del mutation via sequential CasPlus editing. FIG. 8A. Schematic showing the targeted exon with CFTR F508del mutation from the wild-type individual (upper sequence) and CFTR F508del patients (lower sequence). The deleted nucleotides in CFTR-F508del patients are marked with red dash line. FIG. 8B. Schematic showing the sequences of the guide RNA, PAM and single-stranded oligodeoxynucleotides (ssODN) template used for generation of CFTR-F508del knock-in HEK293T cell line. FIG. 8C. Schematic demonstrating four potential strategies for correction of CFTR mutation F508del via CasPlus. One-step insertion of 3 bps creates an allele with missense mutation. Two- or three-steps incorporation of 3 bps by sequential CasPlus editing corrects the mutant allele. FIG. 8D. Guide RNAs and PAM sequences used for sequential correction of CFTR-F508del mutation. TS32 is designed to target CFTR-F508del mutant allele, TS33 is utilized to target an intermediate mutant product with insertions of a thymidine, and TS34 and TS36 are used to target an intermediate mutant product with insertion of AT or TT, respectively. FIG. 8E. Indels profiles and frequency induced by Cas9 editing (including Cas9-NG-WT and Cas9-NG-F916del) and CasPlus editing with guide RNA TS32 in CFTR-F508del HEK293T cells. CasPlus editing predominantly promoted the generation of 1-bp and 2-bp insertions. Cas9-NG is a Cas9 variants that recognize NGN PAM sequences FIG. 8F-FIG. 8G. Indels profiles and frequency induced by two-step sequential CasPlus editing. The editing outcomes from CasPlus-V1 and CasPlus-V2 in combination with either guide RNA TS32 and TS33 or guide RNA TS32 and 34 was shown in FIG. 8F. The editing outcomes from CasPlus-V3.1 and CasPlus-V4.1 with combinations of guide RNA either TS32 and 33 or TS32 and 34 is shown in FIG. 8G. FIG. 8H. Indels profiles and frequency induced by sequential CasPlus editing with combinations of guide RNA either TS32, TS33 and TS34 or TS32, TS33 and TS35. FIG. 8I. The pattern of 3-bp insertions detected in FIG. 8F and FIG. 8G. FIG. 8J. The pattern of 3-bp insertion detected in FIG. 8H. For FIG. 8A the sequence for WT is: 5′-GCACCATTAAAGAAAATATCATCTTTGG-3′ (SEQ ID NO: 9); the sequence for F508del is: 5′-GCACCATTAAAGAAAATATCATTGG-3′ (SEQ ID NO: 10). For FIG. 8B the sequence for CFTR-WT is: 5′-CACCATTAAAGAAAATATCATCTTTGG-3′ (SEQ ID NO: 11); the sequence for ssODN is: 5′-CCAATGATATTTTCTTTAATGGTGC-3′ (SEQ ID NO: 12). For FIG. 8C the sequence for WT is: AATATCATCTTTGGTGTT (SEQ ID NO: 13); the sequence for missense is: AATATCATCATTGGTGTT (SEQ ID NO: 14); the sequence for corrected are AATATCATATTTGGTGTT (SEQ ID NO: 15) and AATATCATTTTTGGTGTT (SEQ ID NO: 16). For FIG. 8D the sequences for CFTR-F508del are: Top: 5′-ATTAAAGAAAATATCATTGGTGTTTCCTATGATGA-3′ (SEQ ID NO: 17); Bot: 5′-TCATCATAGGAAACACCAATGATATTTTCTTTAAT-3′ (SEQ ID NO: 18); the sequences for CFTR-F508del+T are: Top: 5′-ATTAAAGAAAATATCATTTGGTGTTTCCTATGATGA-3′ (SEQ ID NO: 19); Bot: 5′-TCATCATAGGAAACACCAAATGATATTTTCTTTAAT-3′(SEQ ID NO: 20); the sequences for CFTR-F508del+AT are: Top: 5′-ATTAAAGAAAATATCATATTGGTGTTTCCTATGATGA-3′ (SEQ ID NO: 21); Bot: 5′-TCATCATAGGAAACACCAATATGATATTTTCTTTAAT-3′(SEQ ID NO: 22); the sequences for CFTR-F508del+TT are: Top: 5′-ATTAAAGAAAATATCATTTTGGTGTTTCCTATGATGA-3′ (SEQ ID NO: 23); Bot: 5′-TCATCATAGGAAACACCAAAATGATATTTTCTTTAAT-3′ (SEQ ID NO: 24).

FIGS. 9A-9H. Repression of on-target balanced chromosomal translocations between two chromosomes by CasPlus editing. FIG. 9A. CasPlus editing represses Cas9-mediated chromosomal translocations. FIG. 9B. Schematic illustrating the generation of ROS1-CD74 or CD74-ROS1 fused chromosomes. FIG. 9C. Representative gel images showing ROS1-CD74 and CD74-ROS1 translocations in HEK293T cells during Cas9, CasPlus-V1, or CasPlus-V2 editing. HEK293T cells were transfected with vectors expressing Cas9 (with T2A-GFP) and guide RNAs targeting genes ROS1 and CD74 individually or alone with vectors expressing T4-WT or T4-D219A. Transfected Cells were sorted into GFP⁺ population 72 hr post-transfection and subjected to DNA isolation immediately. DMD is a control for intensity normalization. FIG. 9D. Normalized quantification of data in C. Band intensity obtained from Cas9-edited cells is set as 1. Value and error bar reflects mean±SEM of n=3 replicate. FIG. 9E. Frequency of indels at ROS1 and CD74 individual sites in HEK293T cells during Cas9, CasPlus-V1, or CasPlus-V2 editing. Value and error bar reflects mean±SEM of n=3 replicate. FIG. 9F. Representative gel images demonstrating the ROS1-CD74 and CD74-ROS1 translocations in iPSC cells. Induced pluripotent stem cells were transfected with vectors expressing Cas9 (with T2A-GFP) and guide RNAs targeting genes ROS1 and CD74 alone with vectors expressing T4-WT or T4-D219A. Transfected Cells were sorted into GFP⁺ population 72 hr post-transfection and subjected to DNA isolation immediately FIG. 9G. Normalized quantification of data in FIG. 9F. FIG. 9H. Frequency of indels at ROS1 and CD74 individual sites in iPSCs. For FIG. 9C, the sequence for Chr6-Chr5: ROS1-CD74 is: 5′-GAAGCAAAGGG-3′ (SEQ ID NO: 25); the sequence for Chr5-Chr6: CD74-ROS1 is: 5′-GAAGTACAGGCT-3′ (SEQ ID NO: 26).

FIGS. 10A-10D. Repression of on-target balanced chromosomal translocations among multiple chromosomes by CasPlus editing. FIG. 10A. Schematic illustrating the balanced translocations among the genes PDCD1, TRBC1/2, and TRAC. FIG. 10B. Representative gel images demonstrating the balanced translocations detected in HEK293T cells during Cas9, CasPlus-V1, or CasPlus-V2 editing. HEK293T cells were transfected with vectors expressing Cas9 (with T2A-GFP) and guide RNAs targeting genes PDCD1, TRBC1/2 and TRAC alone with vectors expressing T4-WT or T4-D219A. Transfected Cells were sorted into GFP⁺ population 72 hr post-transfection and subjected to DNA isolation immediately. Bands with expected size (red arrowhead) were purified, TA-cloned and sequenced. Balanced translocation of Chr14:Chr2, TRAC-PDCD1 was undetectable by PCR. FIG. 10C. Normalized quantification of data in FIG. 10B. Value and error bar reflects mean±SEM of n=2 replicate. FIG. 10D. Frequency of out-of-frame and in-frame indels at four individual sites in HEK293T cells during Cas9, CasPlus-V1, or CasPlus-V2 editing. Value and error bar reflects mean±SEM of n=2 replicate. For FIG. 10B, the sequence for Chr2-Chr7: PDCD1-TRBC1 is: 5′-CCCAGACCCAGG-3′ (SEQ ID NO: 27); the sequence for Chr2-Chr7: PDCD1-TRBC2: is: 5′-AGCCCACCCAGG-3′ (SEQ ID NO: 28); the sequence for Chr2-Chr14: PDCD1-TRAC: is 5′-CCCAGATCTATG-3′ (SEQ ID NO: 29); the sequence for Chr7-Chr2: TRBC1/2-PDCD1 is: 5′-AGTGGACGACTG-3′ (SEQ ID NO: 30); the sequence for Chr7-Chr14: TRBC1/2-TRAC is: 5′-AGTGGATCTATG-3′ (SEQ ID NO: 31); the sequence for Chr14-Chr7: TRAC-TRBC1 is: 5′-TGAGGTCCCAGG-3′ (SEQ ID NO: 32); the sequence for Chr14-Chr7: TRAC-TRBC2 is: 5′-TGAGGTCCCAGG-3′ (SEQ ID NO: 33).

FIGS. 11A-11C. Represses of on-target unbalanced chromosomal translocations among multiple chromosomes by CasPlus editing. FIG. 11A. Schematic illustrating 6 types of unbalanced inter-chromosomal translocations among the genes PDCD1, TRBC1/2, and TRAC. FIG. 11B. Gel images demonstrating the unbalanced translocations induced by Cas9, CasPlus-V1, or CasPlus-V2 with guide RNAs targeting PDCD1, TRBC1/2, and TRAC. Bands with expected size (red arrowhead) were purified, TA-cloned and sequenced. FIG. 11C. Quantitation of the data in FIG. 11B. Value and error bar reflects mean±SEM of n=2 replicate. For FIG. 11B, the sequence for Chr2-Chr7 (No centromere) (PDCD1-TRBC1) is: 5′-GCGCCCAGGATA-3′(SEQ ID NO: 34); the sequence for Chr2-Chr7 (No centromere) (PDCD1-TRBC2) is: 5′-CCAGTCCCCAGG-3′(SEQ ID NO: 35); the sequence for Chr2-Chr14 (No centromere) (PDCD1-TRAC) is: 5′-CCAGTCTATGGA-3′(SEQ ID NO: 36); the sequence for Chr2-Chr7 (Dicentromere) (TRBC1/2-PDCD1) is: 5′-AGTGGATCTGGG-3′ (SEQ ID NO: 37); the sequence for Chr2-Chr14 (Dicentromere) (TRAC-PDCD1) is: 5′-TGAGGTTCTGGG-3′ (SEQ ID NO: 38); the sequence for Chr7-Ch14 (No centromere) (TRBC1-TRAC) is: 5′-CCTGGGGACTTC-3′ (SEQ ID NO: 39); the sequence for Chr7-Chr14 (No centromere) (TRBC2-TRAC) is: 5′-CCTGGGCTATGG-3′ (SEQ ID NO: 40); the sequence for Chr7-Chr14 (Dicentromere) (TRBC1/2-TRAC) is: 5′-AGTGGAACCTCA-3′(SEQ ID NO: 41).

FIG. 12. Features of CasPlus editing. CasPlus editing utilizes T4 DNA polymerase to fill in the Cas9-created overhangs, thereby biasing insertions over small or large deletions. CasPlus editing can also repress chromosomal translocations that potentially occur between either on-target and off-target site during Cas9-mediated single site editing or different on-target genes during multiplex gene editing.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The disclosure includes all polynucleotide and all amino acid sequences that are identified herein by way of a database entry. Such sequences are incorporated herein as they exist in the database on the filing date of this application or patent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included. The nucleotide and amino acid sequences described herein include all contiguous segments of the described nucleotide sequences that are at least 10 nucleotides or 10 amino acids in length.

As used in the specification and the appended claims, the singular forms “a” “and” and “the” include plural referents unless the context clearly dictates otherwise. Ranges and other values may be expressed herein as from “about” or “approximately” one particular value, and/or to “about” or “approximately” another particular value. When values are expressed as approximations by the use of the antecedent “about” or “approximately” it will be understood that the particular value forms another embodiment. The term “about” and “approximately” in relation to a numerical value encompasses variations of +/−10%, to +/−1%.

The disclosure includes all steps and reagents such as proteins and nucleic acids, and all combinations of steps reagents, described herein, and as depicted on the accompanying figures. The described steps may be performed as described, including but not necessarily sequentially.

In certain embodiments, amino acid sequences described herein may refer to a sequence that lacks an initial Met. For example, for the T4 DNA polymerase amino acid sequence, the mutation described at position 219 may in the amino acid sequence at position 218 due to the expression vector cloning process.

In embodiments, the disclosure provides variations of a T4 DNA polymerase/Cas9 system referred to as “CasPlus.” The variations of the CasPlus system are referred to herein as CasPlus-V1, which comprises among other described components a combination of Cas9-WT and T4-WT. The Cas9 and the described variants refer to the amino acid sequence of Cas9 produced by Streptococcus pyogenes (“SpCas9”). CasPlus-V2 comprises among other described components a combination of Cas9-WT and T4-D219A. CasPlus-V3 and V4 comprises among other described components combinations of Cas9 variants as further described herein and either T4-WT or T4-D219A, respectively. T4 DNA polymerases described herein are MS2-targeted. CasPlus-V3 and V4 may comprise subcategories based on the Cas9 variant that is used. Cas9 variants F916P, F916del, R919P and Q920P are referred to herein as V3.1, V3.2. V3.3 and V3.4, respectively, in CasPlus-V3. For CasPlus-V4, the described Cas9 variants are described as V4.1, V4.2, V4.3 and V4.4, respectively. “F916del” means a deletion of the F residue at position 916. The described Cas9 variants may also be used in a composition, method, and system of the disclosure with an RB69 DNA polymerase, wherein the RB69 polymerase optionally comprises a mutation of D222, and wherein the mutation is optionally D222A.

As illustrated by the Examples and figures, the described systems are used to precisely model and correct mutations by producing predictable indels formed following Cas9 cleavage. The system creates indels in a DNA repair template free manner. The described systems have improved properties relative to other gene editing systems in that CasPlus editing in comparison to standard Cas9 editing is they reduce unwanted changes to on-target and off-target sites, such as large deletions, translocations, and other chromosomal rearrangements. In embodiments, the described systems and methods reduce microhomology-mediated end-joining. Instead, in embodiments, the indel is produced via non-homologous end joining (NHEJ) which is at least in part facilitated by a described T4 DNA polymerase that is a component of the system.

By designing the described CasPlus system and described variants with an enhanced probability of generating preferred indels, the disclosure includes generation of isogenic patient cells with greater efficiency as compared to traditional homology directed repair (HDR) methods. The presently provided results demonstrate the utility of CasPlus system and its variants with designed gRNAs for traits beyond cleavage efficiency and gene specificity and the capacity to harness predictable indel formation for modeling and correction of a wide-range of indel-based diseases. Thus, the present disclosure provides compositions and methods for producing precise insertion and/or deletions in a guide RNA targeted segment of a chromosome. Accordingly, the disclosure in certain embodiments is used to produce indels. Indels comprise an insertion or deletion of 1, 2, 3, 4, or 5, nucleotides, with concomitant changes on the complementary strand, thus resulting in an insertion or deletion of 1-10 base pairs (bp), inclusive. The indel may comprise any desired change by using one or more suitable guide RNAs in conjunction with the protein complexes as further described herein.

In non-limiting embodiments, the indel is produced within a protein coding segment of a chromosome, at a splice junction, in a promoter, in an enhancer element, or at any other location wherein generation of an indel is desirable, provided a suitable proto adjacent motif (PAM) is proximal to the location of the indel. In embodiments, the indel corrects a mutation that is associated with a condition or disorder. In embodiments, the indel corrects a frameshift mutation, a missense mutation, or a nonsense mutation. In embodiments, the indel changes a codon for at least one amino acid in a protein coding sequence, and thus may correct a mutation in an exon to a normal (e.g., non-disease associated) exon. In embodiments, a homozygous indel may be produced. In embodiments, the indel corrects a deleterious mutation that is a component of a monogenic disorder, e.g., a disorder caused by variation in a single gene. In embodiments, the monogenic disorder is an X-linked disorder. In non-limiting embodiments, the monogenic disorder is any of sickle cell anemia, cystic fibrosis, Huntington disease, Tay-Sachs disease, phenylketonuria, mucopolysaccharidoses, lysosomal acid lipase deficiency, glycogen storage diseases, galactosemia, Hemophilia A, Rett's syndrome, or any form of muscular dystrophy, such as Duchenne muscular dystrophy (DMD). In a non-limiting embodiment, the indel corrects a mutation in the human dystrophin gene. In embodiments, the indel corrects a mutation (including but not necessarily limited to a deletion) in the human dystrophin gene that is comprised by one or more human dystrophin gene exons 2-10 or 45-55, each inclusive. In embodiments, the indel corrects one or more out-frame mutations within exons by producing a single base pair insertion. Thus, the disclosure includes exon reshaping, such as reframing an out of frame reading frame. In embodiments, the indel restores functional dystrophin expression in cells in which the mutation is corrected. In non-limiting embodiments, the disclosure provides for introducing a 1 bp insertion in human dystrophin gene exon 43, 45, 49, 51 or 53. The amino acid sequence of human dystrophin and the sequence of the gene encoding human dystrophin is known in the art, such as via NCBI Gene ID: 1756, including all accession numbers therein, and in NCBI accession number NG 012232, which are incorporated herein as it exists in the NCBI database as of the effective filing date of this application or patent.

In non-limiting embodiments, the disclosure provides for correcting a mutation of a gene that is correlated with cystic fibrosis. In an embodiment, the disclosure provides for correcting a F508del in the gene that encodes the cystic fibrosis transmembrane conductance regulator protein (CFTR). The amino acid sequence of CFTR is known in the art and is available under NCBI Reference sequence: NP 000483.3, from which the amino acid sequence is incorporated herein as it exists in the NCBI database as of the effective filing date of this application or patent. The disclosure includes all polynucleotide sequences encoding the CFTR protein.

In embodiments, the disclosure provides fusion proteins that facilitate the association a DNA polymerase with a wild type of variant of a Cas nuclease, as further described herein. In embodiments, the fusion proteins comprise an MS2 domain and a T4 DNA polymerase domain, representative sequences of variations of which are described herein.

In embodiments, the disclosure provides for more frequent indel production relative to a control. In embodiments, the control comprises an indel production value obtained by using a DNA polymerase that is not a T4 DNA polymerase or an RB69 DNA polymerase that includes the described mutations, or a described system that includes a wild type Cas9 sequence, or a protein that does not exhibit nuclease activity, such as a detectable protein, non-limiting examples of which are provided herein and comprise Green Fluorescent Protein (GFP), but other proteins may be used, such a mCherry.

In embodiments, if the DNA polymerase is provided as a fusion protein, the fusion protein may comprise one or more ribosomal skipping sequences, which are also referred to in the art as “self-cleaving” amino acid sequences. These are typically about 18-22 amino acids long. Any suitable sequence can be used, non-limiting example of which include T2A, comprising the amino acid sequence: EGRGSLLTCGDVEENPGP (SEQ ID NO: 42); P2A, comprising the amino acid sequence ATNFSLLKQAGDVEENPGP (SEQ ID NO: 43); E2A, comprising the amino acid sequence QCTNYALLKLAGDVESNPGP (SEQ ID NO: 44); and F2A, comprising the amino acid sequence VKQTLNFDLLKLAGDVESNPGP (SEQ ID NO: 45).

In embodiments, the fusion proteins may comprise linking amino acids (e.g., linkers) that separate one or more protein domains. The linker is typically at least two amino acids long, and may include a GS sequence, but other sequences may be used. In embodiments, the linker is from 3-100 amino acids in length. In embodiments, a linker sequences comprises or consists of a “GS” sequence. In embodiments, the linker comprises or consists of the sequence SAGGGGSGGGGSGGGGSG (SEQ ID NO: 46).

In embodiments, a fusion protein of the disclosure includes one or more nuclear localization signals, representative and non-limiting examples of which are provided herein. In general, for eukaryotic purposes, a nuclear localization signal comprises one or more short sequences of positively charged lysines or arginines.

In non-limiting embodiments, the disclosure provides a fusion protein that comprise an MS2 segment and a DNA polymerase segment, which may also include the aforementioned linking amino acids, nuclear localization signals, and ribosome skipping/self-cleaving sequences. A segment means a section of the described protein that contains contiguous amino acid sequences. In embodiments, the segment is of sufficient length to retain the function of protein to participate in the described method and is thus a functional segment. In embodiments, a segment comprises a contiguous segment of a described protein that includes contiguously 80%-99% of a described amino acid sequence.

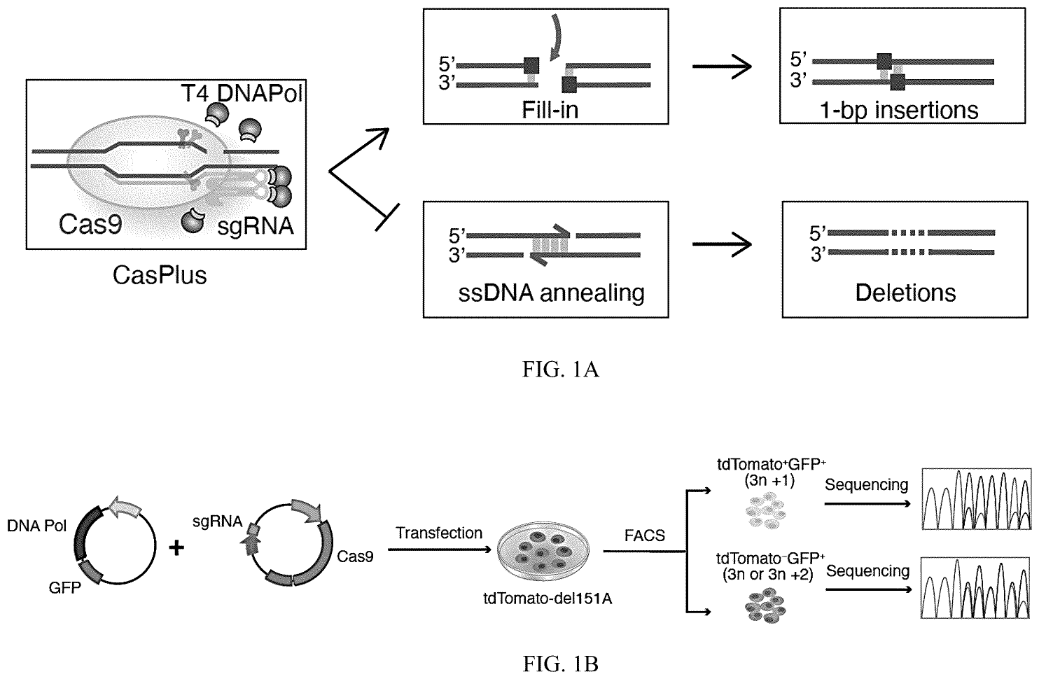

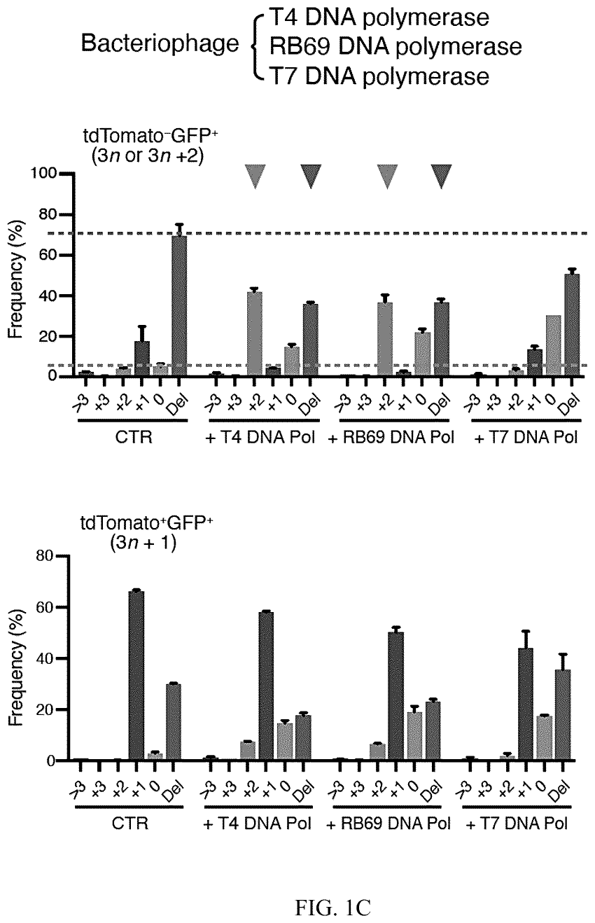

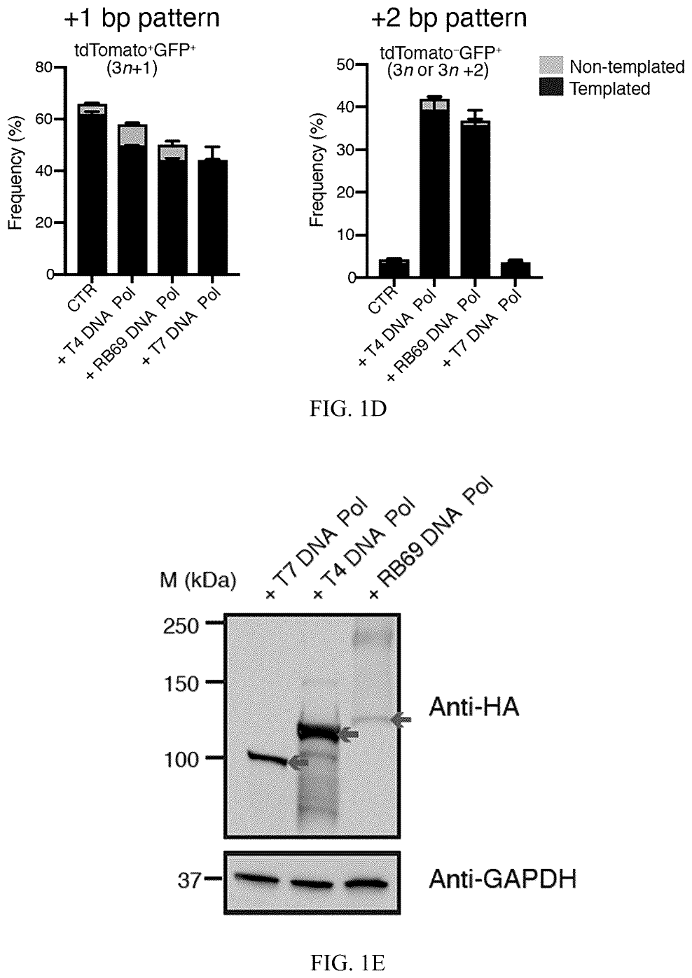

In an embodiment, whether present in a fusion protein or not, the DNA polymerase is T4 DNA polymerase, but other DNA polymerases that enable the fill in of overhang maybe used, such as T7 DNA polymerase, may be used. We have demonstrated that the following DNA polymerases do not function in the described system: DNA polymerase lambda, DNA polymerase Mu, DNA polymerase Beta, yeast derived DNA polymerase 4, bacteria derived DNA polymerase I and Klenow fragment all do not exhibit adequate or any detectable function (see, for example, FIGS. 1D-1E).

In an embodiment, the T4 DNA polymerase comprises the sequence:

(SEQ ID NO: 47)
KEFYISIETVGNNIVERYIDENGKERTREVEYLPTMFRHCKEESKYKDIY
GKNCAPQKFPSMKDARDWMKRMEDIGLEALGMNDFKLAYISDTYGSEIVY
DRKFVRVANCDIEVTGDKFPDPMKAEYEIDAITHYDSIDDRFYVFDLLNS
MYGSVSKWDAKLAAKLDCEGGDEVPQEILDRVIYMPFDNERDMLMEYINL
WEQKRPAIFTGWNIEGFDVPYIMNRVKMILGERSMKRFSPIGRVKSKLIQ
NMYGSKEIYSIDGVSILDYLDLYKKFAFTNLPSFSLESVAQHETKKGKLP
YDGPINKLRETNHQRYISYNIIDVESVQAIDKIRGFIDLVLSMSYYAKMP
FSGVMSPIKTWDAIIFNSLKGEHKVIPQQGSHVKQSFPGAFVFEPKPIAR
RYIMSFDLTSLYPSIIRQVNISPETIRGQFKVHPIHEYIAGTAPKPSDEY
SCSPNGWMYDKHQEGIIPKEIAKVFFQRKDWKKKMFAEEMNAEAIKKIIM
KGAGSCSTKPEVERYVKFSNATAITIFGQVGIQWIARKINEYLNKVCGTN
DEDFIAAGDTDSVYVCVDKVIEKVGLDRFKEQNDLVEFMNQFGKKKMEPM
IDVAYRELCDYMNNREHLMHMDREAISCPPLGSKGVGGFWKAKKRYALNV
YDMEDKRFAEPHLKIMGMETQQSSTPKAVQEALEESIRRILQEGEESVQE
YYKNFEKEYRQLDYKVIAEVKTANDIAKYDDKGWPGFKCPFHIRGVLTYR
RAVSGLGVAPILDGNKVMVLPLREGNPFGDKCIAWPSGTELPKEIRSDVL
SWIDHSTLFQKSFVKPLAGMCESAGMDYEEKASLDFLFG.

Any suitable MS2 sequence may be used that provides binding sites to MS2 bacteriophage coat protein. [Seminars in Virology 8, 176-185 (1997), article No. VI970120, from which the disclosure is incorporated herein by reference]. In an embodiment, a fusion protein of the disclosure comprises an MS2 sequence which comprises the sequence:

(SEQ ID NO: 48)
MASNFTQFVLVDNGGTGDVTVAPSNFANGVAEWISSNSRSQAYKVTCSVR
QSSAQKRKYTIKVEVPKVATQTVGGVELPVAAWRSYLNMELTIPIFATNS
DCELIVKAMQGLLKDGNPIPSAIAANSGIY.

Any suitable MS2 bacteriophage coat protein sequence may be used, including any MS2 bacteriophage coat protein sequence having between 80-99.99% sequence identity to the above sequence and that provides requisite binding sites to MS2 RNA aptamers. In an embodiment, the fusion protein comprises a first linker sequence that comprises the sequence SAGGGGSGGGGSGGGGSG (SEQ ID NO: 46). In an embodiment, the fusion protein comprises a second linker sequence that comprises the sequence GS.

In an embodiment, the fusion protein comprises one or more nuclear localization signals. In an embodiment, the one or more nuclear localization signals (NLSs) comprise the sequence:

| (SEQ ID NO: 49) | |
|---|---|
| | GPKKKRKVAAA |

In an embodiment, a system of the disclosure comprises a fusion protein comprising in an N->C terminal direction a contiguous polypeptide that comprises: an MS2 protein segment, a first linker, a first NLS, a T4 DNA polymerase segment, a second linker sequence, and a second NLS. This construct may also be used as a control to demonstrate improved properties of the described CasPlus variants. A representative construct is as follows, and as further described below:

(SEQ ID NO: 50)
MASNFTQFVLVDNGGTGDVTVAPSNFANGVAEWISSNSRSQAYKVTCSVRQSSA
QKRKYTIKVEVPKVATQTVGGVELPVAAWRSYLNMELTIPIFATNSDCELIVKA
MQGLLKDGNPIPSAIAANSGIYSAGGGGSGGGGSGGGGSGPKKKRKV

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GSGPKKKRKVAAA,

wherein the MS2 sequence is shown in bold, the linker sequences are shown in italics, the NLS sequences are shown in enlarged font, and the T4 DNA sequence is shown in bold and italics.

In an embodiment, the disclosure provides a fusion protein encoded by a sequence comprising or consisting of the following nucleic acid sequences, and/or encoding any of the following amino acid sequences as annotated:

T4-D219A Protein sequence
MS2-Linker-NLS-T4-D219A-NLS
(SEQ ID NO: 51)
MASNFTQFVLVDNGGTGDVTVAPSNFANGVAEWISSNSRSQAYKVTCSVRQSSA
QKRKYTIKVEVPKVATQTVGGVELPVAAWRSYLNMELTIPIFATNSDCELIVKA
MQGLLKDGNPIPSAIAANSGIYSAGGGGSGGGGSGGGGSGPKKKRKV

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

PKKKRKVAAA.
T4-D219A DNA sequences
MS2-Linker-NLS-T4-D219A-NLS
(SEQ ID NO: 52)
atggcttcaaactttactcagttcgtgctcgtggacaatggtgggacaggggat
gtgacagtggctccttctaatttcgctaatggggtggcagagtggatcagctcc
aactcacggagccaggcctacaaggtgacatgcagcgtcaggcagtctagtgcc
cagaagagaaagtataccatcaaggtggaggtccccaaagtggctacccagaca
gtgggcggagtcgaactgcctgtcgccgcttggaggtcctacctgaacatggag
ctcactatcccaattttcgctaccaattctgactgtgaactcatcgtgaaggca
atgcaggggctcctcaaagacggtaatcctatcccttccgccatcgccgctaac
tcaggtatctacagcgctggaggaggtggaagcggaggaggaggaagcggagga
ggaggtagcggacctaagaaaaagaggaaggtg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cctaagaaaaagaggaaggtg.
RB69 DNA polymerase protein sequences
MS2-Linker-NLS-T4-D219A-NLS
(SEQ ID NO: 53)
MASNFTQFVLVDNGGTGDVTVAPSNFANGVAEWISSNSRSQAYKVTCSVRQSSA
QKRKYTIKVEVPKVATQTVGGVELPVAAWRSYLNMELTIPIFATNSDCELIVKA
MQGLLKDGNPIPSAIAANSGIYSAGGGGSGGGGSGGGGSGPKKKRKV

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

PKKKRKVAAA.
RB69 DNA polymerase DNA sequences
MS2-Linker-NLS-RB69-NLS
(SEQ ID NO: 54)
atggcttcaaactttactcagttcgtgctcgtggacaatggtgggacaggggat
gtgacagtggctccttctaatttcgctaatggggtggcagagtggatcagctcc
aactcacggagccaggcctacaaggtgacatgcagcgtcaggcagtctagtgcc
cagaagagaaagtataccatcaaggtggaggtccccaaagtggctacccagaca
gtgggcggagtcgaactgcctgtcgccgcttggaggtcctacctgaacatggag
ctcactatcccaattttcgctaccaattctgactgtgaactcatcgtgaaggca
atgcaggggctcctcaaagacggtaatcctatcccttccgccatcgccgctaac
tcaggtatctacagcgctggaggaggtggaagcggaggaggaggaagcggagga
ggaggtagcggacctaagaaaaagaggaaggtg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cctaagaaaaagag
gaaggtg.
RB69-D222A Protein sequences
MS2-Linker-NLS-RB69-D222A-NLS
(SEQ ID NO: 55)
MASNFTQFVLVDNGGTGDVTVAPSNFANGVAEWISSNSRSQAYKVTCSVRQSSA
QKRKYTIKVEVPKVATQTVGGVELPVAAWRSYLNMELTIPIFATNSDCELIVKA
MQGLLKDGNPIPSAIAANSGIYSAGGGGSGGGGSGGGGSGPKKKRKV

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

PKKKRKVAAA.
RB69-D222A DNA sequences
MS2-Linker-NLS-RB69-D222A-NLS
(SEQ ID NO: 56)
atggcttcaaactttactcagttcgtgctcgtggacaatggtgggacaggggat
gtgacagtggctccttctaatttcgctaatggggtggcagagtggatcagctcc
aactcacggagccaggcctacaaggtgacatgcagcgtcaggcagtctagtgcc
cagaagagaaagtataccatcaaggtggaggtccccaaagtggctacccagaca
gtgggcggagtcgaactgcctgtcgccgcttggaggtcctacctgaacatggag
ctcactatcccaattttcgctaccaattctgactgtgaactcatcgtgaaggca
atgcaggggctcctcaaagacggtaatcctatcccttccgccatcgccgctaac
tcaggtatctacagcgctggaggaggtggaagcggaggaggaggaagcggagga
ggaggtagcggacctaagaaaaagaggaaggtg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cctaagaaaaagag
gaaggtg.
T7 DNA polymerase Protein sequence
MS2-Linker-NLS-T7-DNA-Pol-NLS
(SEQ ID NO: 57)
MASNFTQFVLVDNGGTGDVTVAPSNFANGVAEWISSNSRSQAYKVTCSVRQSSA
QKRKYTIKVEVPKVATQTVGGVELPVAAWRSYLNMELTIPIFATNSDCELIVKA
MQGLLKDGNPIPSAIAANSGIYSAGGGGSGGGGSGGGGSGPKKKRKV

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

PKKKRKVAAA.
T7 DNA polymerase DNA sequence
MS2-Linker-NLS-T7-DNA-Pol-NLS
(SEQ ID NO: 58)
atggcttcaaactttactcagttcgtgctcgtggacaatggtgggacaggggat
gtgacagtggctccttctaatttcgctaatggggtggcagagtggatcagctcc
aactcacggagccaggcctacaaggtgacatgcagcgtcaggcagtctagtgcc
cagaagagaaagtataccatcaaggtggaggtccccaaagtggctacccagaca
gtgggcggagtcgaactgcctgtcgccgcttggaggtcctacctgaacatggag
ctcactatcccaattttcgctaccaattctgactgtgaactcatcgtgaaggca
atgcaggggctcctcaaagacggtaatcctatcccttccgccatcgccgctaac
tcaggtatctacagcgctggaggaggtggaagcggaggaggaggaagcggagga
ggaggtagcggacctaagaaaaagaggaaggtg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cctaagaaaaagaggaaggtg.

Any suitable amino sequence having between 80-99.99% sequence identity to the above sequence, and all other sequences described herein, wherein the sequence has the requisite DNA polymerase activity to facilitate NHEJ or other DNA edits and that provides requisite binding sites to MS2 bacteriophage coat protein, are included in this disclosure.

Any suitable nucleic acid sequence may be used in this invention that encodes any of the foregoing amino sequences having between 80-99.99% sequence identity, wherein the amino acid sequence has the requisite DNA polymerase activity to facilitate the described DNA editing and that provides requisite binding sites to MS2 bacteriophage coat protein, are included in this disclosure.

A utility of the described fusion protein is the “tagging” of the T4 DNA polymerase with the MS2 protein segment. MS2 tagging is used to recruit the MS2 protein and another protein to which the MS2 is linked, such as a Cas enzyme, to RNA sequences that comprise a tetraloop and stem loop 2 of, for example, a guide RNA. These features protrude outside of a Cas9-gRNA ribonucleoprotein complex, with the distal 4 base pairs (bp) of each stem free of interactions with Cas9 amino acid side chains. The tetraloop and stem loop 2 allow the addition of protein-interacting RNA aptamers to facilitate the recruitment of effector domains to the Cas9 complex (e.g. [Nature volume 517, pages 583-588(2015)], from which the disclosure is incorporated herein by reference. Thus, the described system is used to recruit the described T4 DNA or described RB69 polymerase to guide RNA comprising MS2 binding domains, and a Cas enzyme. Other protein recruiting system may be used, such SunTag, a system for recruiting multiple protein copies to a polypeptide scaffold. [Cell. 2014 Oct. 23; 159(3): 635-646, from which the disclosure is incorporated herein by reference].

In embodiments, the DNA polymerase catalyzes the synthesis of DNA in the 5′->3′ direction to create the indel after cleavage by the Cas enzyme. In embodiments, the described system inhibits microhomology-mediated end joining. In embodiments, the disclosure provides for creating a 1˜2 base pairs staggered ends with a 5′ overhang, which allow precise and predictable insertions of 1˜2 nucleotide(s) that are identical to the sequence(s) 4˜5 base pairs upstream of the PAM, by DNA polymerase-mediated fill in over the staggered ends.

In specific and non-limiting embodiments, the Cas comprises a Cas9, such as Streptococcus pyogenes (SpCas9). Derivatives of Cas9 are known in the art and may also be used with the described DNA polymerase. Such derivatives may be, for example, smaller enzymes that Cas9, and/or have different proto adjacent motif (PAM) requirements. In a non-limiting embodiment, the Cas enzyme may be Cas12a, also known as Cpf1, or SpCas9-HF1, or HypaCas9, or xCas9, or Cas9-NG, or SpG, or SpRY.

In a non-limiting embodiment, the DNA endonuclease may be transposon-associated TnpB. The reference sequence of S. pyogenes is available under GenBank accession no. NC_002737, with the cas9 gene at position 854757-858863. The S. pyogenes Cas9 amino acid sequence is available under number is NP_269215. These sequences are incorporated herein by reference as they were provided on the priority date of this application or patent.

The Cas enzyme is provided with one or more suitable guide RNAs, which may be referred to as a “targeting RNA” or “targeting RNAs.” Representative guide RNAs and used in the Examples are provided in Table 1. Table 1 also provides target sites that correspond to the guide RNAs.

In general, the targeting RNA is provided such that it includes suitable MS2 binding sites. In an embodiment, a suitable guide RNA comprises a sequence that is: NNNNNNNNNNNNNNNNNNNNguuuuagagcuaggccaacaugaggaucacccaugucugcagggccu agcaaguuaaaauaaggcuaguccguuaucaacuuggccaacaugaggaucacccaugucugcagggccaaguggcacc gagucggugcuuuuuuu (SEQ ID NO: 59), wherein the bold uppercase letter represents the selected spacer, and the bold lowercase letters represent the MS2 loops to which the T4-MS2 fusion protein binds. However, the present disclosure unexpectedly reveals that the MS2 binding sites are not necessarily required for the CasPlus system to function. Thus, the guide RNA may be provided with or without MS2 binding sites. In embodiments, the DNA polymerase may be provided without any MS2 binding sites. Thus, in non-limiting embodiments, the DNA polymerase may be provided as DNA polymerase that is not a segment of a fusion protein.

Any of the described components may be introduced into cells using any suitable route and form. In embodiments, the disclosure provides for use of one or more plasmids or other suitable expression vectors that encode the targeting RNA, and/or the described proteins. In embodiments, the disclosure provides RNA-protein complexes, e.g., RNAPs.

In embodiments, a viral expression vector may be used for introducing one or more of the components of the described system. Viral expression vectors may be used as naked polynucleotides, or may comprises viral particles. In embodiments, the expression vector comprises a modified viral polynucleotide, such as from an adenovirus, a herpesvirus, or a retrovirus, such as a lentiviral vector. In embodiments, one or more components of the described of CasPlus system variants may be delivered to cells using, for example, a recombinant adeno-associated virus (AAV) vector. Adeno-associated virus (AAV) is a replication-deficient parvovirus, the single stranded DNA genome of which is about 4.7 kb in length including 145 nucleotide inverted terminal repeat (ITRs). The nucleotide sequence of the AAV serotype 2 (AAV2) genome is presented in Ruffing el al., J Gen Virol, 75: 3385-3392 (1994). Cis-acting sequences directing viral DNA replication (rep), encapsidation/packaging and host cell chromosome integration are contained within the ITRs. As the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as an expression cassette, with the rep and cap proteins provided in trans. The sequence located between ITRs of an AAV vector genome is referred to herein as the “payload”. A recombinant AAV (rAAV) may therefore contain up to about 4.7 kb, 4.6 kb, 4.5 kb or 4.4 kb of unique payload sequence. Following infection of a target cell, protein expression and replication from the vector requires synthesis of a complementary DNA strand to form a double stranded genome. This second strand synthesis represents a rate limiting step in transgene expression. AAV vectors are commercially available, such as from TAKARA BIO® and other commercial vendors, and may be adapted for use with the described systems, given the benefit of the present disclosure. In embodiments, for producing AAV vectors, plasmid vectors may encode all or some of the well-known rep, cap and adeno-helper components. In certain embodiments, the expression vector is a self-complementary adeno-associated virus (scAAV). In scAAV vectors, the payload contains two copies of the same transgene payload in opposite orientations to one another, i.e. a first payload sequence followed by the reverse complement of that sequence. These scAAV genomes are capable of adopting either a hairpin structure, in which the complementary payload sequences hybridize intramolecularly with each other, or a double stranded complex of two genome molecules hybridized to one another. Transgene expression from such scAAVs is much more efficient than from conventional AAVs, but the effective payload capacity of the vector genome is halved because of the need for the genome to carry two complementary copies of the payload sequence. Suitable scAAV vectors are commercially available, such as from CELL BIOLABS, INC.® and can be adapted for use in the presently provided embodiments when given the benefit of this disclosure.

In this specification, the term “rAAV vector” is generally used to refer to vectors having only one copy of any given payload sequence (i.e. a rAAV vector is not an scAAV vector), and the term “AAV vector” is used to encompass both rAAV and scAAV vectors. AAV sequences in the AAV vector genomes (e.g. ITRs) may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11 and AAV PHP.B. The nucleotide sequences of the genomes of the AAV serotypes are known in the art. For example, the complete genome of AAV-1 is provided in GenBank Accession No. NC_002077; the complete genome of AAV-2 is provided in GenBank Accession No. NC 001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); AAV PHP.B is described by Deverman et al., Nature Biotech. 34(2), 204-209 and its sequence deposited under GenBank Accession No. KU056473.1.

In embodiments, non-viral delivery systems may be used for introducing one or more of the components of the described system. Non-viral tools including hydrodynamic injection, electroporation and microinjection. Hydrodynamic injection can systemically deliver CasPlus variants into targeted tissues, including but not necessarily limited to liver. To permeate endothelial and parenchymal cells, hydrodynamic injections require a high injection volume, speed and pressure that limit central nervous system therapies. Electroporation and microinjection can be used for germline editing or embryo manipulation. Chemical vectors, such as lipids and nanoparticles, are widely used for delivery. Cationic lipids interact with negatively charged DNA and the cell membrane, protecting the DNA and cellular endocytosis. DNA nanoparticles, such as, are potential delivery strategies. DNA conjugated to gold nanoparticles (CRISPR-gold) complexed with cationic endosomal disruptive polymers can deliver the described CasPlus variants into animal cells.

In embodiments, expression vectors, proteins, RNPs, polynucleotides, and combinations thereof, can be provided as pharmaceutical formulations. A pharmaceutical formulation can be prepared by mixing the described components with any suitable pharmaceutical additive, buffer, and the like. Examples of pharmaceutically acceptable carriers, excipients and stabilizers can be found, for example, in Remington: The Science and Practice of Pharmacy (2005) 21st Edition, Philadelphia, PA. Lippincott Williams & Wilkins, the disclosure of which is incorporated herein by reference. Further, any of a variety of therapeutic delivery agents can be used, and include but are not limited to nanoparticles, lipid nanoparticle (LNP), fusosomes, exosomes, and the like. In embodiments, a biodegradable material can be used. In embodiments, poly(lactide-co-galactide) (PLGA) is a representative biodegradable material, but it is expected that any biodegradable material, including but not necessarily limited to biodegradable polymers. As an alternative to PLGA, the biodegradable material can comprise poly(glycolide) (PGA), poly(L-lactide) (PLA), or poly(beta-amino esters). In embodiments, the biodegradable material may be a hydrogel, an alginate, or a collagen. In an embodiment the biodegradable material can comprise a polyester a polyamide, or polyethylene glycol (PEG). In embodiments, lipid-stabilized micro and nanoparticles can be used.

In embodiments, a combination of proteins, and a combination one or more proteins and polynucleotides described herein, may be first assembled in vitro and then administered to a cell or an organism.

The cells into which the described systems are introduced are not particularly limited, and may include postmitotic adult tissues, which are considered to be refractory to HDR, such as for example, heart and skeletal cells. The disclosure is not necessarily limited to such cells, and may also be used with, for example, with totipotent, pluripotent, multipotent, or oligopotent stem cells. In embodiments, the cells are neural stem cells. In embodiments, the cells are hematopoietic stem cells. In embodiments, the cells are leukocytes. In embodiments, the leukocytes are of a myeloid or lymphoid lineage.

In embodiments, the cells are embryonic stem cells, or adult stem cells. In embodiments, the cells are epidermal stem cells or epithelial stem cells. In embodiments, the cells are muscle precursor cells, such as quiescent satellite cells, or myoblasts, including but not necessarily limited to skeletal myoblasts and cardiac myoblasts.

In some examples the lymphocytes are T cells, In certain examples a modified T cell is also modified such that it expresses a chimeric antigen receptor (CAR). In embodiments, the cells are natural killer (NK) or natural killer T cells, which may also be modified to express a CAR.

As is known in the art, T cells may be modified by using canonical Cas systems to increase safety by knocking out PDCD1, TRBC1, TRBC2, and TRAC. In some embodiments, a described system is used to create an indel in one more of the genes PDCD1, TRBC1, TRBC2, and TRAC, in T cells. The disclosure demonstrates that using a described system inhibits translocation events. Previous Cas systems used to produce modifications to these genes increase the risk of translocation. The disclosure demonstrates that using a described system lowers the risk of translocation, and therefore provides an approach to more safely creating modified cells, including but not necessarily modified T cells that will be used in a CAR format. In embodiments, use of a described CasPlus system reduces balanced or unbalanced translocations. In embodiments, use of a described CasPlus system reduces intra- or inter-chromosomal translocation. In embodiments, use of a described CasPlus system reduces large deletions caused by previous systems. In embodiments, a large deletion is a deletion of at least 500 nucleotides.

Thus, the present invention provides for creating indels using a described CasPlus system as an alternative to previously available Cas systems or other targeted nucleases where a knock-out or other disruption or modification of a gene is desirable, but creates a risk of translocation. Accordingly, in embodiments, the disclosure provides for using a described CasPlus system as an alternative to any other guide-directed or other targeted nuclease that is used to concurrently modify one or more loci. In embodiments, the disclosure provides an alternative to modification using any type of Cas enzyme, a zinc finger nuclease, or a transcription activator-like effector nuclease (TALEN), or a transposon-based DNA editing system. In embodiments, a described CasPlus system is used to modify at least two genetic locations, while reducing risk of translocation. As such, the described CasPlus systems can be used with 2, 3, 4, or more guide RNAs concurrently or sequentially to modify more than one locus, while lowering the risk of translocation events.

In embodiments, the disclosure includes obtaining cells from an individual, modifying the cells ex vivo using a system as described herein, and reintroducing the cells or their progeny into the individual or an immunologically matched individual for prophylaxis and/or therapy of a condition, disease or disorder, as described above. In embodiments, the cells modified ex vivo as described herein are autologous cells. In embodiments, the cells are mammalian cells. The disclosure is thus suitable for a wide range of human, veterinary, experimental animal, and cell culture uses.

The following Examples are intended to illustrate but not limit the disclosure.

### Examples

Identification of T4 and RB69 DNA Polymerase as Proteins that Favor CasPlus Editing.

T4 DNA polymerase-mediated CasPlus editing system can enhance the fill-in of the 5′ overhangs created by Cas9, leading to an enhancement of 1-bp insertions, while simultaneously inhibiting the annealing of micro-homologies (MHs) at the double-strand break (DSB) sites, thereby reducing deletions generated by the microhomology-mediated end-joining (MMEJ) repair pathway (FIG. 1A). We investigated whether overexpression of other bacteriophage-derived DNA polymerases impact Cas9-mediated indel outcomes in tdTomato reporter cell lines. We first constructed MS2-tagged DNA polymerase expression vectors optimized for human codons. We subsequently transfected vectors that either expressed Cas9, GFP or tdTomato-sgRNA alone, or in combination with a distinct MS2-tagged DNA polymerase, into tdTomato reporter cell lines. Transfected cells were sorted into populations expressing either only GFP (tdTomato⁻/GFP⁺) or both tdTomato and GFP (tdTomato⁺/GFP⁺), for genomic DNA isolation and sequencing (FIG. 1B). High-throughput sequencing (HTS) of tdTomato⁻/GFP⁺ populations indicated that overexpression of T4 and RB69 DNA polymerase, which have 74% amino acid similarity⁽²⁷⁾, resulted in an approximate 6-fold increase in the frequency of 2-bp insertions, at the expense of the frequency of deletions (FIG. 1C). This effect was not observed with overexpression of T7 DNA polymerase⁽²⁸⁾. HTS of tdTomato⁺/GFP⁺ populations revealed similar indel profiles from all treatment groups. Further analysis of insertion patterns showed that >95% of 2-bp insertions in tdTomato⁻/GFP⁺ populations were template-dependent (FIG. 1D). We confirmed that the expression of all DNA polymerases expressed in tdTomato reporter cell lines by Western Blot analysis (FIG. 1E). Synthesis of the results described above indicates that RB69 and T4 DNA polymerase favor the CasPlus editing.

### T4 DNA Polymerase Mutant D219A (T4-D219A) Improves T4 DNA Polymerase-Mediated CasPlus Editing Efficiency.

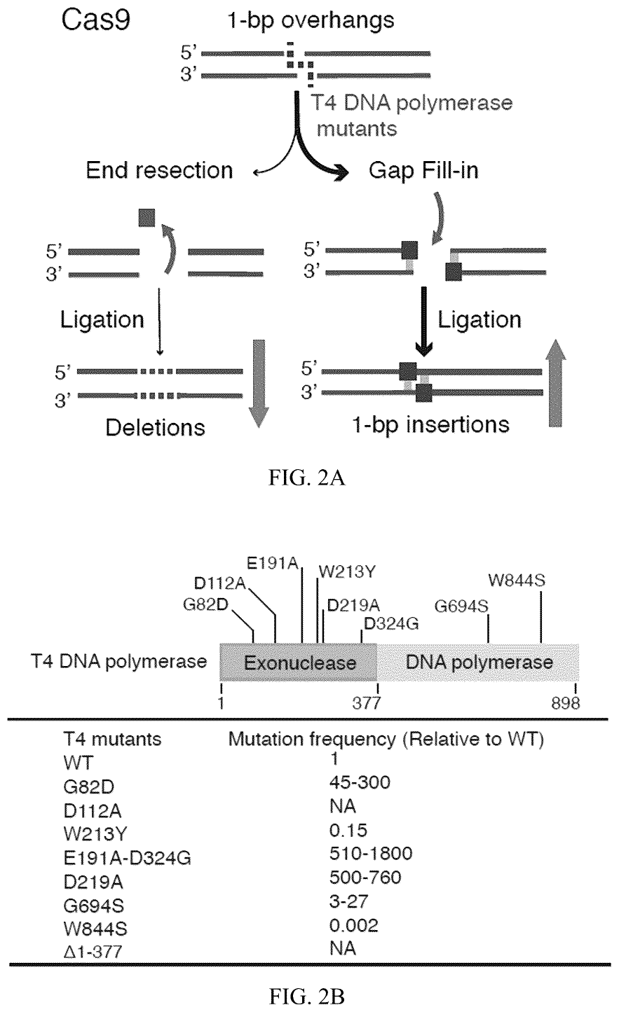

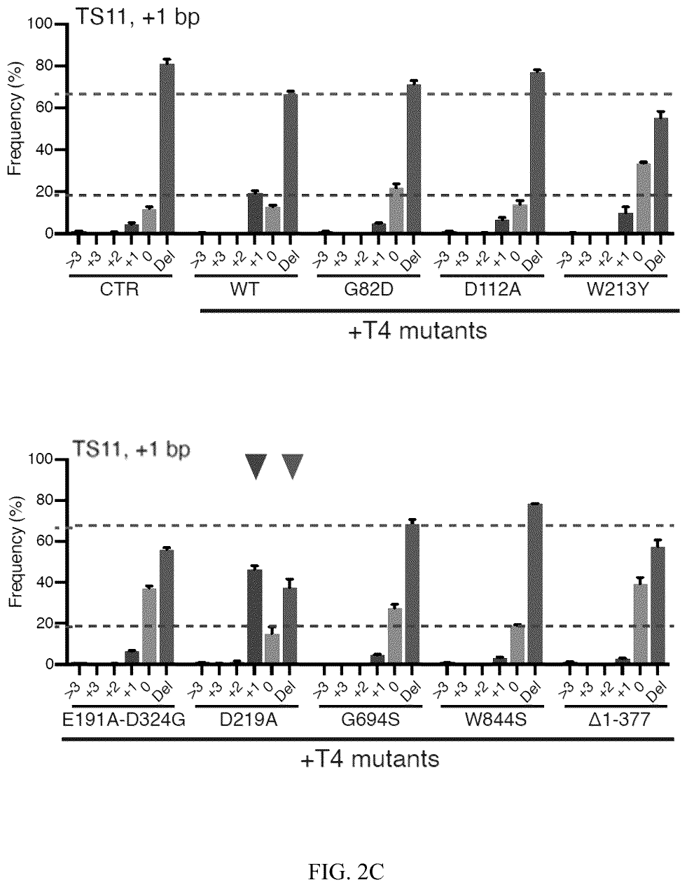

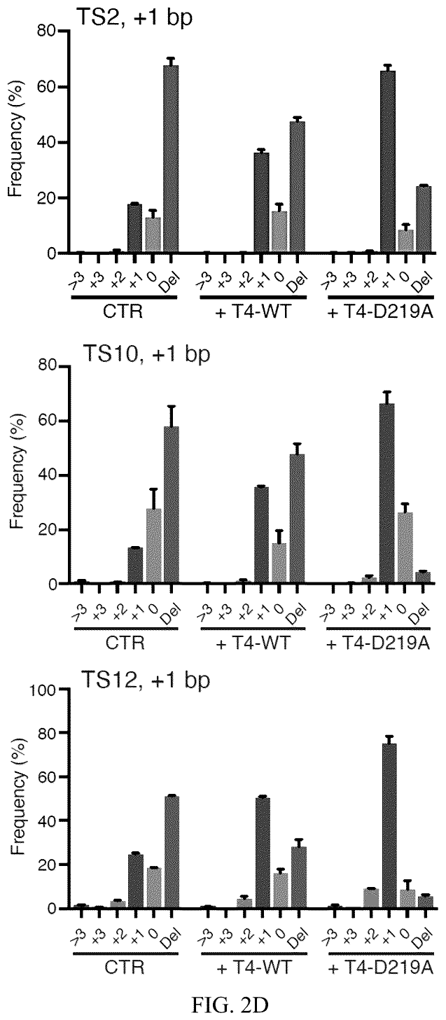

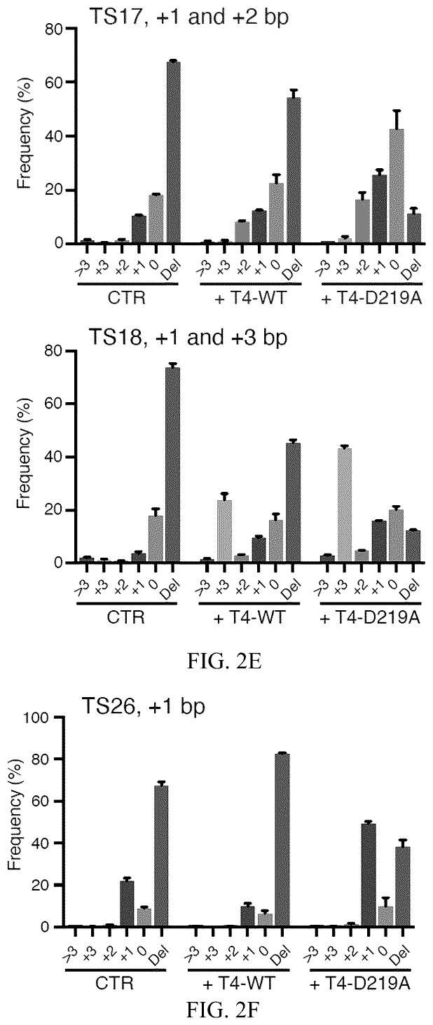

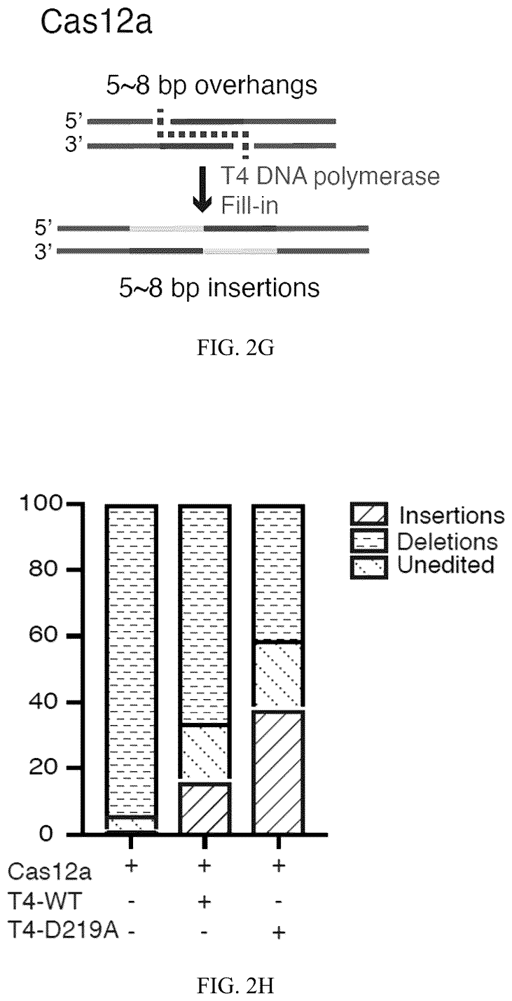

Given that the efficiency of insertions generated by CasPlus editing are highly dependent on the efficiency of filling-in 5′ overhangs via T4 DNA polymerase, we analyzed whether enhancement of T4 DNA polymerase's 5′→3′-polymerase activity or decrement of 3′→5′-exonuclease activity can further increase CasPlus editing efficiency (FIG. 2A). T4 DNA polymerases are multifunctional and can replicate DNA and proofread mis-incorporated nucleotides using an exonuclease domain (FIG. 2B). The 3′-5′ exonuclease activity of T4 DNA polymerase is one of the important determinants of its activity⁽²⁹⁾. Many mutant strains of bacteriophage T4 contain a T4 DNA polymerase with a deficient or highly active exonuclease domain. In the present disclosure, we constructed two T4 mutants (W213Y and W844S) that are associated with decreased DNA mutation rates, five (G82D, D112A, D219A, E191A-D324G and G694S) that increased DNA mutation frequency, and one N-terminus truncation mutant that lacks the 3′-5′ exonuclease domain (delete 1-377 aa⁽²⁴⁻²⁶⁾ (FIG. 2B). To evaluate the efficiency of promoting insertions, we tested target site (TS) 11, which produced a relatively minor increase in 1-bp insertions following overexpression of wild-type T4 DNA polymerase (T4-WT). Strikingly, co-expression of mutant T4-D219A produced a 2.4-fold increase of 1-bp insertions on TS11 in comparison to WT-T4 (FIG. 2C). Conversely, overexpression of other T4 mutants resulted in a decrease of 1-bp insertions on TS11 in comparison to T4-WT.

We further tested the activity of the T4-D219A mutant across other genomic loci. In comparison to T4-WT, T4-D219A mutant led to an additional 1.8 to 2.8-fold increase in 1-bp insertions among all three additional genomic sites tested (FIG. 2D). In comparison to T4-WT, T4-D219A mutant also resulted in a 2-fold increase in 1- and 2-bp insertions at TS17 and a 1.8- and 1.7-fold increase in 3- and 1-bp insertions at TS18 (FIG. 2E). At the TS26, although T4-WT with Cas9 was unable to promote 1-bp insertions, T4-D219A with Cas9 induced a 2.3-fold increase in 1-bp insertions, in comparison to Cas9 alone (FIG. 2F).

Cas12a (also known as Cpf1) is another Cas nuclease that can create 5′ overhangs with 5-8 nucleotides⁽³⁰⁾. We tested whether T4 DNA polymerase can fill in the Cas12a-induced overhangs, thereby resulting in 5-8 nucleotides insertion (FIG. 2G). In contrast, the cleavage site of the Cas12a is distal to the PAM sequence (18˜23-bp from the PAM), therefore Cas12a can re-cut the target sites to generate indels or indels bearing 5-8 nucleotides repeats⁽³¹⁾. Hence, we calculated the frequency of editing products containing insertions but not repeats. HTS results revealed that without T4 DNA polymerase, Cas12a produced editing products with <2% insertions. In contrast, in the presence of T4-WT or T4-D219A, Cas12a produced 17% or 39% insertion frequency, respectively (FIG. 2H). These results revealed that T4-D219A exhibited an improved CasPlus editing efficiency in comparison to T4-WT.

### RB69 DNA Polymerase Mutant D222A (RB69-D222A) Improves RB69 DNA Polymerase-Mediated CasPlus Editing Efficiency.

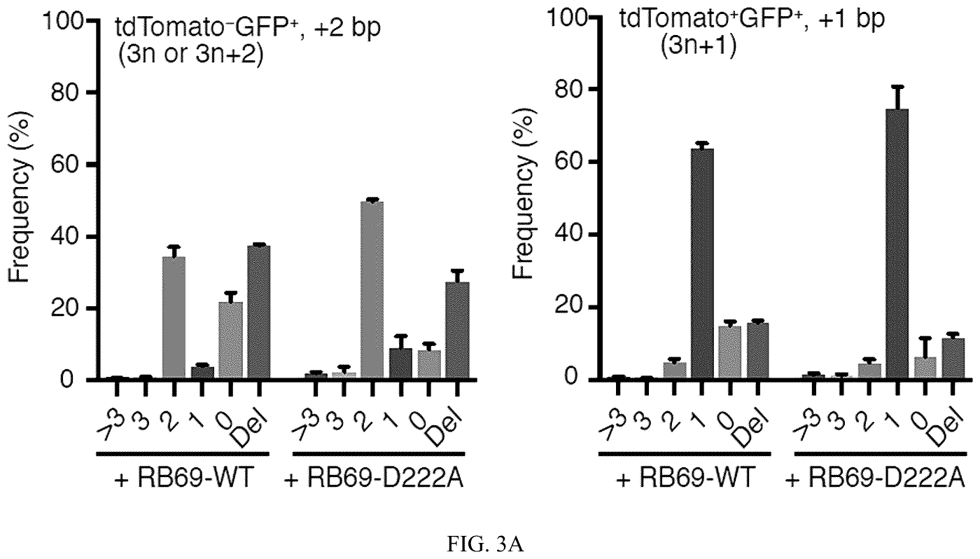

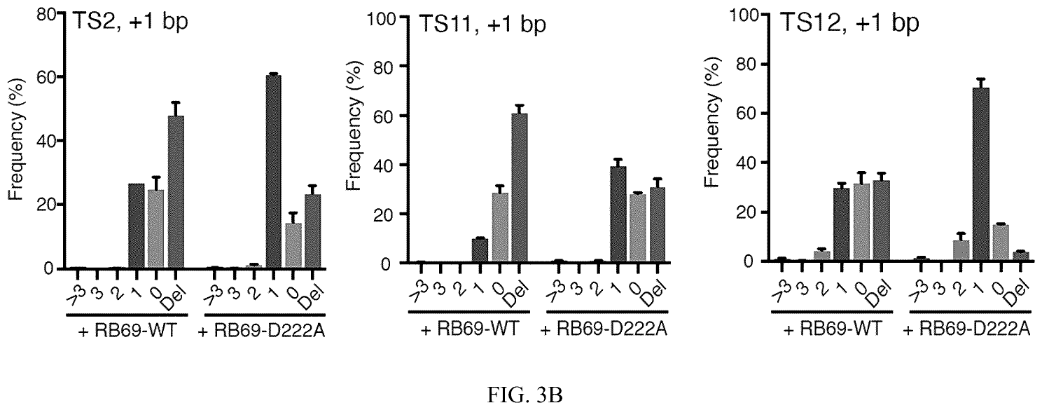

Previous sequence analysis suggested that T4 DNA polymerase residue Asp-219 is analogous to Asp-222 in the wild-type RB69 (RB69-WT) DNA polymerase of RB69 bacteriophage⁽³²⁾. Thus, we investigated the activity of the RB69-D222A mutant across local genomic sites. RB69-D222A increased 2-bp insertions at tdTomato site in comparison to RB69-WT (FIG. 3A). RB69-D222A also led to 2.3-, 3.9- and 2.2-fold increases in 1-bp insertions at TS2, TS11 and TS12, respectively, in comparison to RB69-WT (FIG. 3B). Hence, both the mutations of T4-D219A and RB69-D222A can further improve the 1-bp insertion editing efficiency of CasPlus, in human cells.

Combination of Cas9 Variants and T4 DNA Polymerase Enhances 1-Bp Insertions at Cas9 Target Sites that Predominantly Produce Deletions with Cas9-WT and T4-WT.

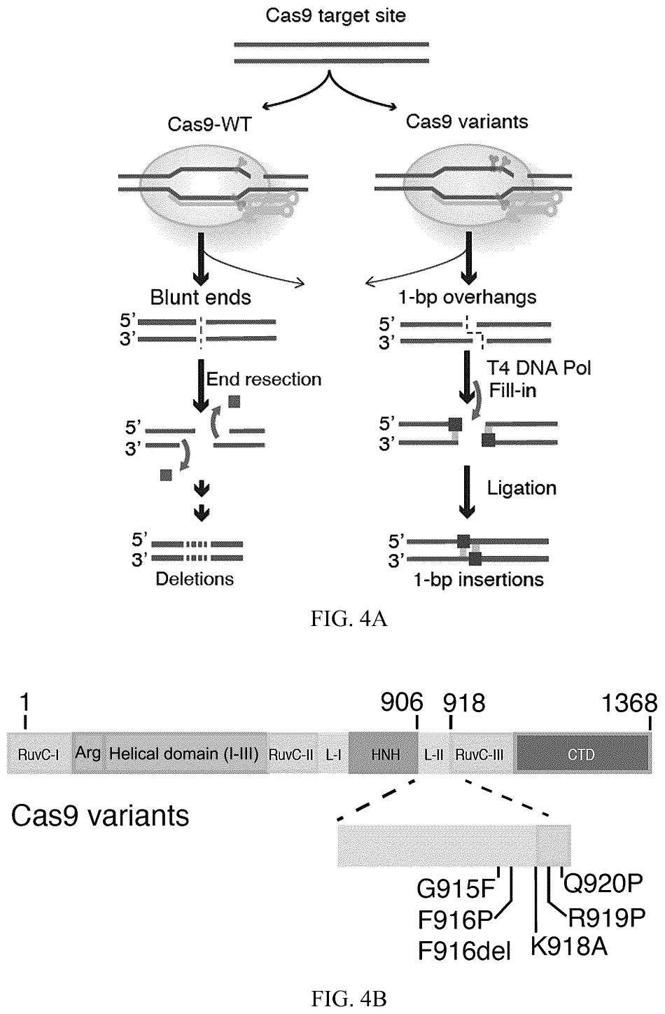

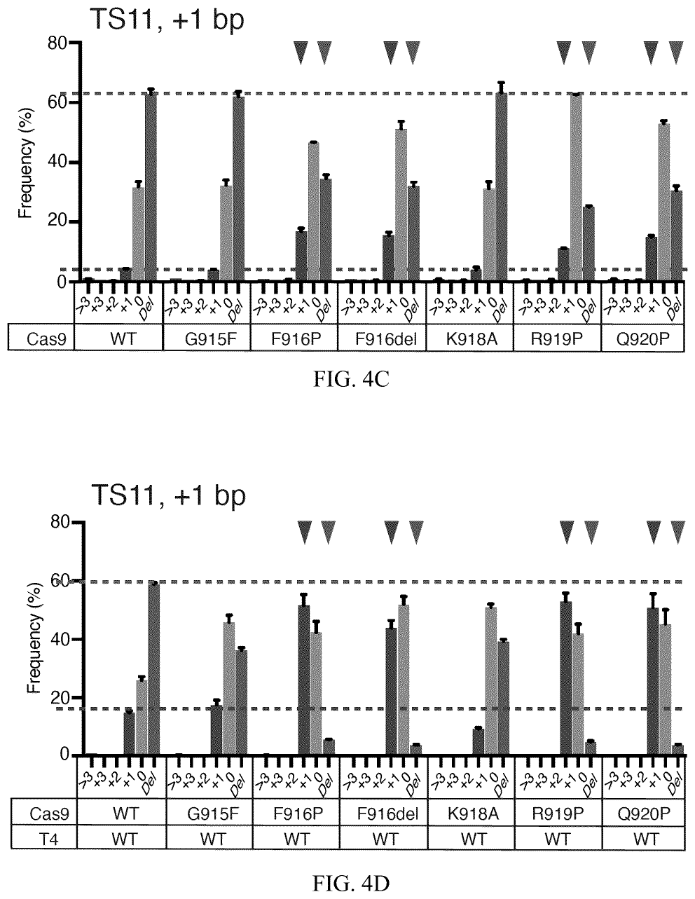

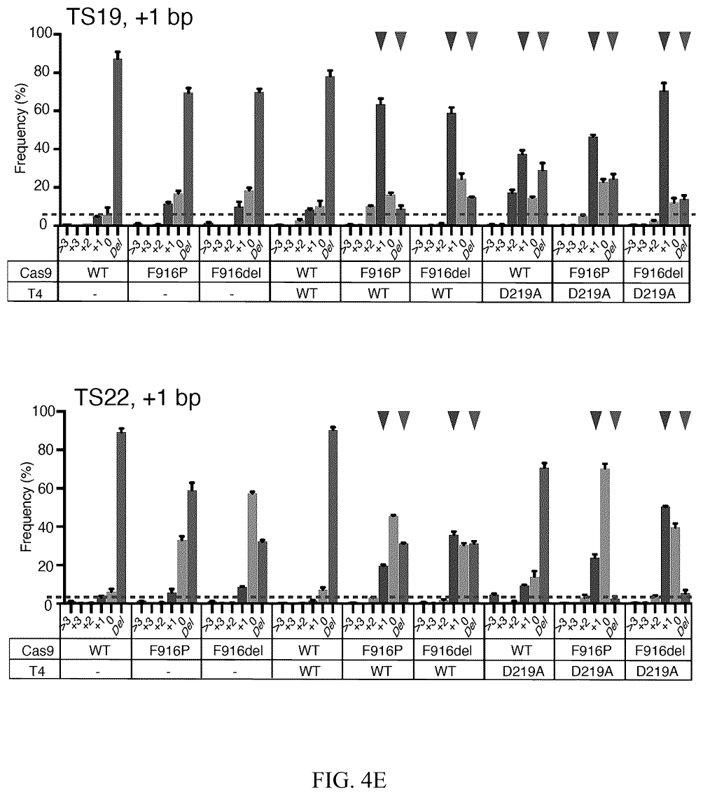

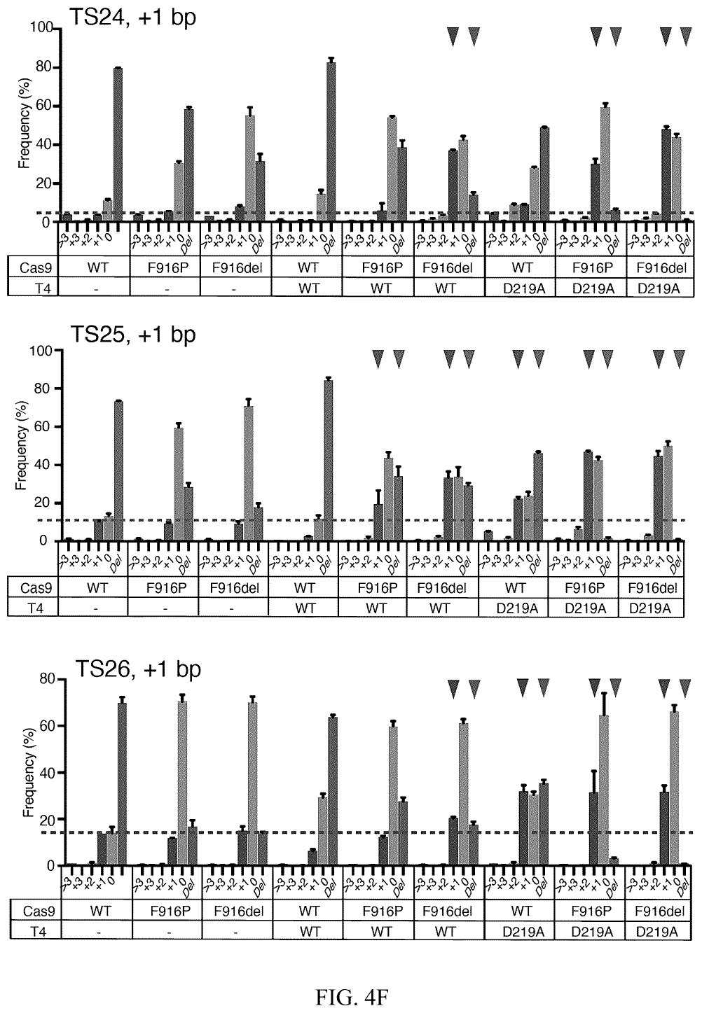

Given that CasPlus editing is correlated with DSB ends with 5′ overhangs, its' editing efficiency is limited by the number and type of staggered ends generated from Cas9 editing. The majority of DSBs induced by Cas9-WT are blunt ends, while some Cas9 variants can be rationally engineered to favor the production of 1-bp overhangs⁽³³⁾. We analyzed whether combining these rationally engineered Cas9 variants with T4 DNA polymerase, could further enhance the frequency of 1-bp insertions (FIGS. 4A-4B). To test this, we transfected cells with either rationally engineered Cas9 variants alone, or in combination with T4-WT, using TS11 as a target. The present disclosure reveals that even though the editing efficiency of Cas9 variants decreased at TS11 in comparison with wild-type Cas9 (Cas9-WT), Cas9 variants F916P, F916del, R919P or Q920P alone led to around 16% of the products with 1-bp insertions whereas Cas9-WT alone produced 4% 1-bp insertions (FIG. 4C). Strikingly, a combination of Cas9 variants F916P, F916del, R919P or Q920P and T4-WT resulted in around 44%-55% 1-bp insertions, whereas the combination of Cas9-WT and T4-WT generated around 15% of edits with 1-bp insertions (FIG. 4D). These results revealed that combination of Cas9 variants and T4 DNA polymerase enables the enhancement of 1-bp insertions. Given that both the deletion of Phe-719 and the mutation of Phe-719 to Pro-719 increased 1-bp insertions in CasPlus editing, we chose to focus the subsequently described examples on Phe-719 mutations.

Our following experiments focused on five target sites, that originally showed insignificant increase in 1-bp insertions in the presence of Cas9-WT and T4-WT. We discovered Cas9 variants F916P and F916del led to an average 4.3-fold or 5.1-fold increase in 1-bp insertions, respectively, in the presence of T4-D219A, across all five target sites in comparison to these Cas9 variants alone. (FIGS. 4E-4F). These results indicate that T4 DNA polymerase can enhance 1-bp insertions when combined with Cas9 variants, at target sites that predominantly produce deletions with Cas9-WT and T4-WT. Overall, the new strategy of combination of Cas9 variants and T4 DNA polymerase expanded the range of their target sites for 1-bp insertions editing results.

Combination of Cas9 Variants and T4 DNA Polymerase Enhances the Production of Longer Insertions (2 to 4 bps)

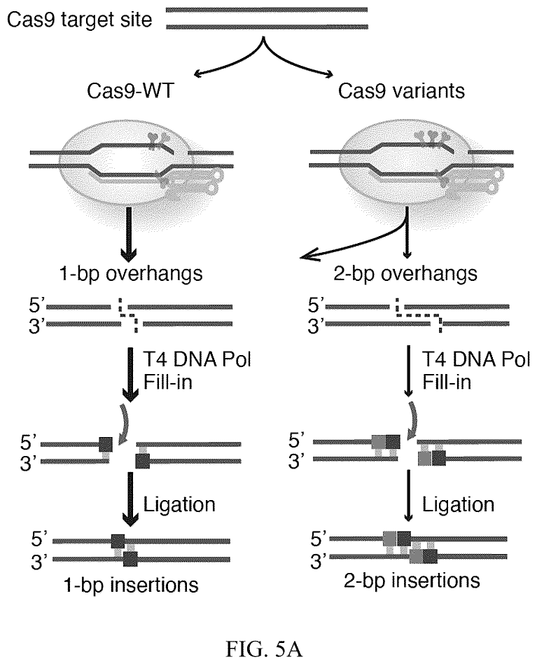

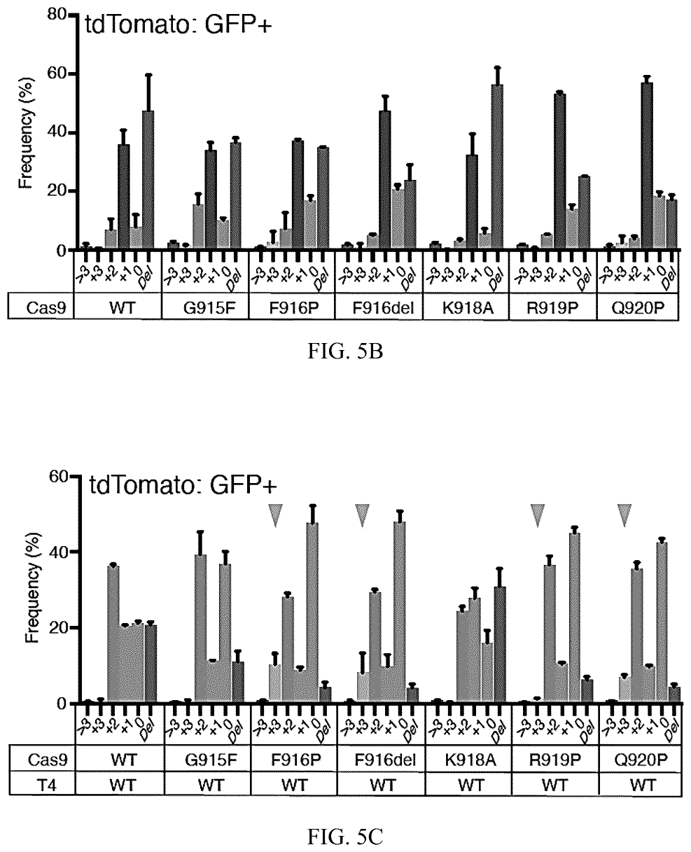

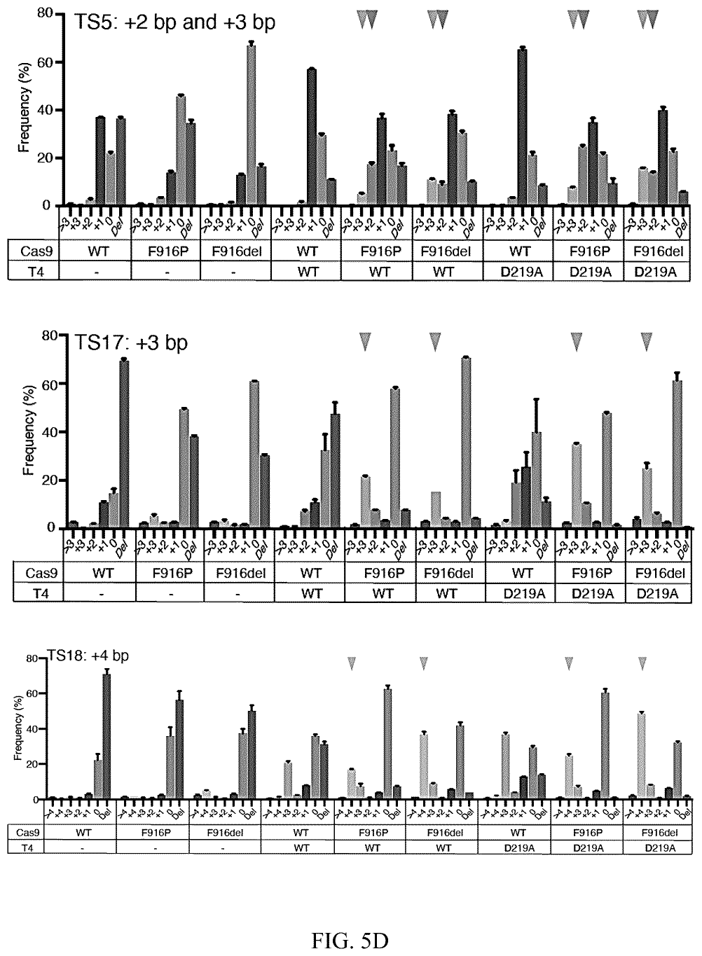

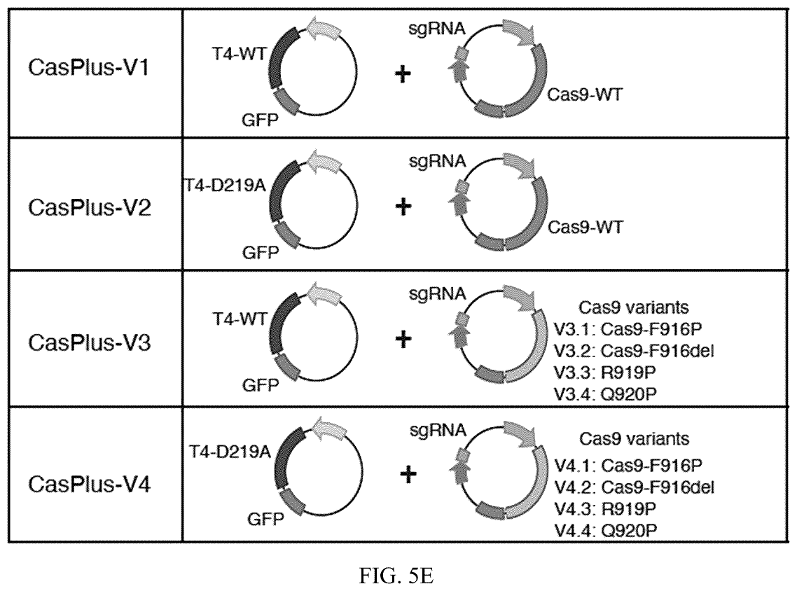

Our previous experiments illustrated that engineered Cas9 variants combined with T4 DNA polymerase can increase the frequency of 1-bp insertions at Cas9 target sites that predominantly produce deletions with Cas9-WT and T4-WT. Therefore, we analyzed whether the same combinations of Cas9 variants and T4 DNA polymerase could increase the frequency of longer insertions, such as 2 to 4-bp insertions, at Cas9 target sites that originally and predominantly generate 1-bp insertions with Cas9-WT and T4-WT (FIG. 5A). We focused on a previous described tdTomato site that predominantly generates 2-bp insertions with Cas9-WT and T4-WT, to determine whether combination of Cas9 variants and T4 DNA polymerase can increase the frequency of 3-bp, or longer insertions. HTS revealed that in the presence of T4 DNA polymerase, Cas9 variants F916P, F916del and Q920P, led to a clear increase in 3-bp insertions in comparison to Cas9-WT, whereas Cas9 variants alone did not alter the frequency of 3-bp insertions (FIGS. 5B-5C).

Next, we investigated the capacity of Cas9-F916P and Cas9-F916del to produce longer insertions at other genomic sites. We used TS5, TS17 and TS18, which predominantly produced 1-bp, 2-bp and 3-bp insertions, respectively, with Cas9-WT and T4-WT. At TS5, Cas9-F916P and Cas9-F916del promoted the generation of 2- or 3-bp insertions when combined with T4 DNA polymerase; At TS17 and TS18, Cas9 variants promoted the generation of 3- and 4-bp insertions, when combined with T4 DNA polymerase (FIG. 5D). These findings led to our conclusion that the combination of Cas9 variants and T4 DNA polymerase can enhance the production of longer insertions (2 to 4 bps).

To elucidate the multi-functionality of the T4 DNA polymerase-mediated CasPlus system, we have categorized it into four versions. CasPlus-V1 is the combination of Cas9-WT and T4-WT. CasPlus-V2 labels the combination of Cas9-WT and T4-D219A. CasPlus-V3 and V4 use the combination of Cas9 variants and either T4-WT or T4-D219A, respectively. CasPlus-V3 and V4 are further divided into subcategories based on the Cas9 variant that is used. Cas9 variants F916P, F916del, R920P and Q920P are named V3.1, V3.2. V3.3 and V3.4, respectively, in CasPlus-V3; or V4.1, V4.2, V4.3 and V4.4, respectively, in CasPlus-V4 (FIG. 5E). All T4 DNA polymerases are MS2-tagged as described before.

### CasPlus System Efficiently Represses On-Target Large Deletions.

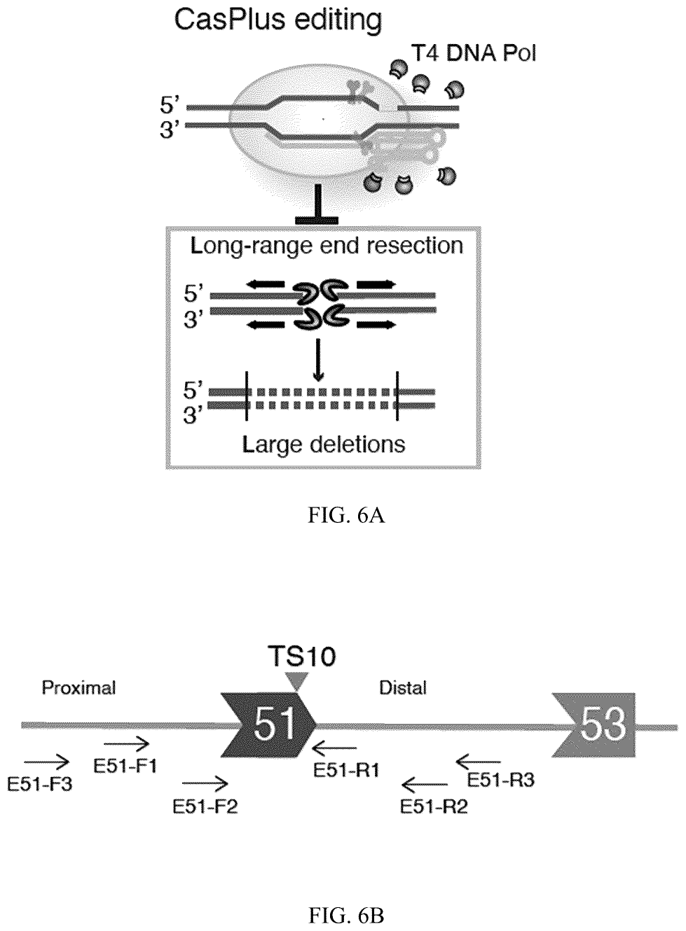

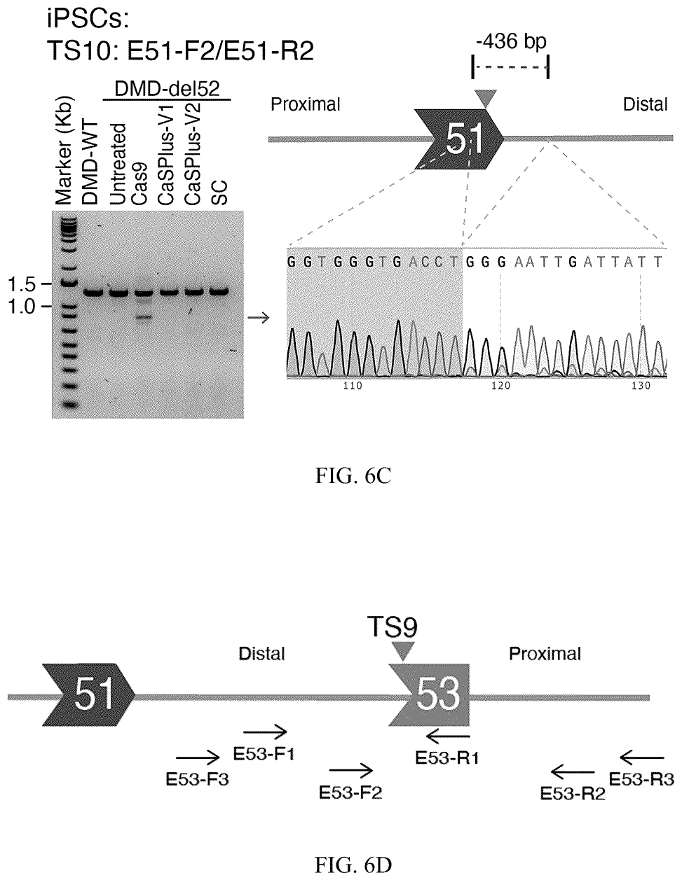

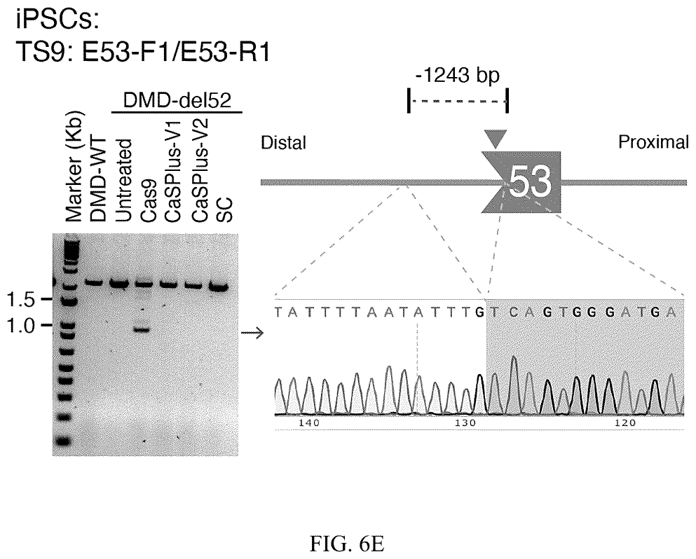

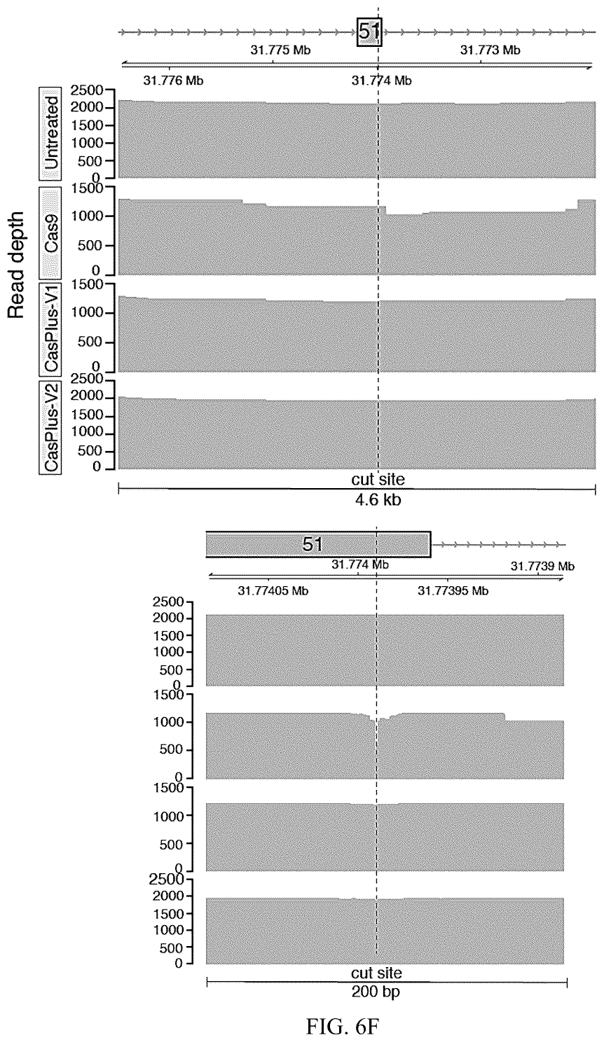

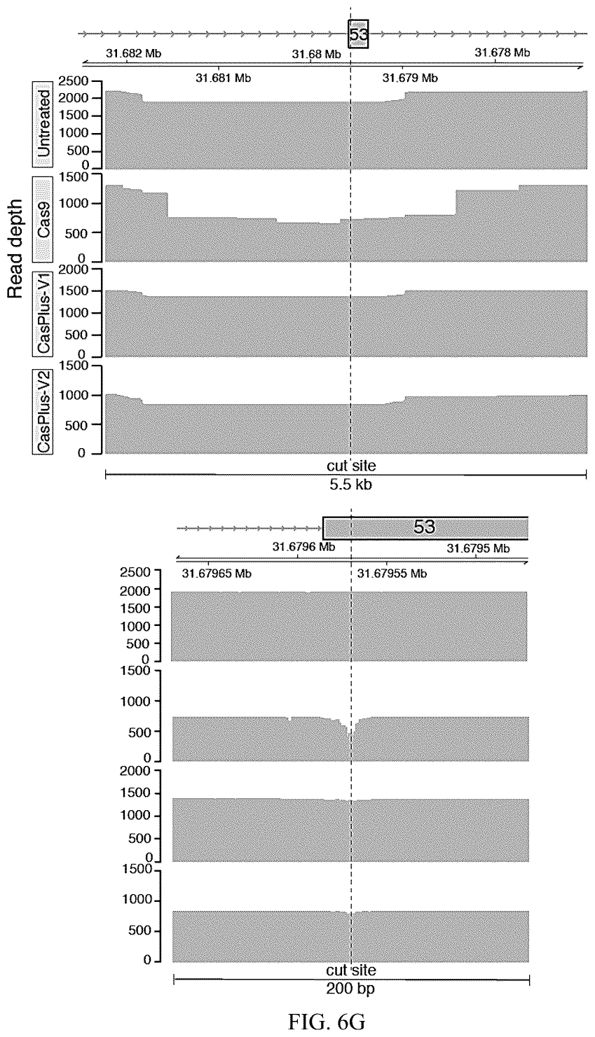

A major concern of regular CRISPR/Cas9 technology in clinical and pre-clinical trials, is the potential for it to generate uncontrollable and unexpected large deletions and complex chromosome rearrangements at Cas9 on-target sites^{(5, 34)}. These large deletions are generally caused by long-range end resection that results from Cas9-induced DSBs (FIG. 6A). Our HTS data, which used PCR amplicons around 300-bp, demonstrated that CasPlus editing predominantly enhanced insertions at the expense of small deletions (<100-bp). We analyzed whether CasPlus editing could also inhibit the production of large deletions (>500-bp) by filling in or binding DSB-induced ends prior to long-range end resection (FIG. 6A). To test this, we evaluated the presence of large deletions at the X-linked DMD locus. We used male iPS cells (iPSCs) to deliver guide RNA targeting TS10 or TS9 on DMD exon 51 or 53, respectively. These guide RNAs were tested in combination with Cas9 and in combination with CasPlus systems. Previous reports have shown that repair of Cas9-induced DSBs leads to asymmetric distribution of on-target indels, favoring changes at the distal, or 5′, region of the PAM⁽³⁵⁾. Therefore, we designed two primer sets to amplify a 1˜2.0 kb PAM distal or proximal region of the target sites from pool of edited cells (FIGS. 6B and 6D). Cas9-edited cells from PAM distal regions were amplified, ran on a gel, and imaged. We observed several lower bands only occurred in Cas9-edited cells in our PCR gel, representing a deletion of around 450 bp and 1.3 kb on TS10 and TS9, respectively. (FIGS. 6C and 6E). We next amplified a ˜5-kb region around the DMD exon 51 and 53 target sites from pools of edited iPSCs and sequenced the PCR amplicons using PacBio sequencing technology. Up to 23.0% of the PacBio reads contained deletions of 0.2-3 kb around the cut site of exon 51 in Cas9-edited cells (FIG. 6F and Table 2). We did not observe this effect in either untreated cells (˜2.0%) or cells edited with CasPlus-V1 (˜3.2%) or -V2 (˜3.5%). In untreated cells, we detected ˜3-kb deletions around DMD exon 53 in 13.2% of the PacBio reads. This result was likely due to a technical problem introduced during the PCR amplification process, as 3-kb deletions of similar scale were observed in all tested samples (Cas9 (11.1%); CasPlus-V1 (9.4%); CasPlus-V2 (14.8%)). On DMD exon 53, Cas9 greatly increased reads with deletions of 0.2-3.5 kb around the cut site in comparison with either untreated cells or those subjected to CasPlus-V1 or -V2 editing (Cas9 (48.9%); CasPlus-V1 (9.5%); CasPlus-V2 (17.4%)) (FIG. 6G and Table 2). Hence, CasPlus-V1- and CasPlus-V2-mediated editing efficiently repressed on-target large deletions.

Enhanced Correction of DMD Exon 52 Deletion in iPSCs Via CasPlus Editing.

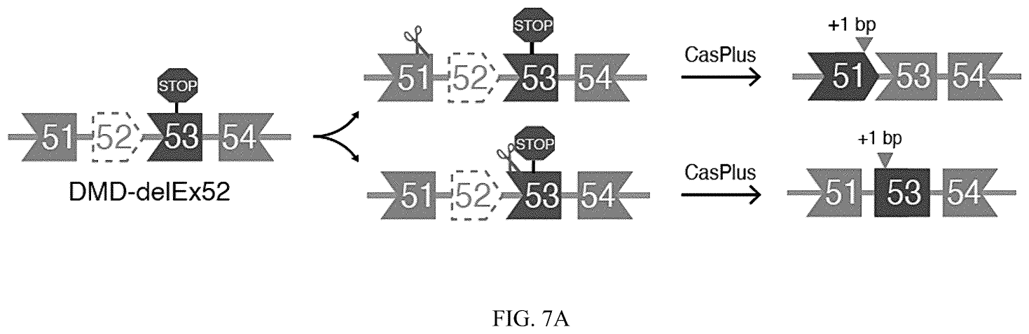

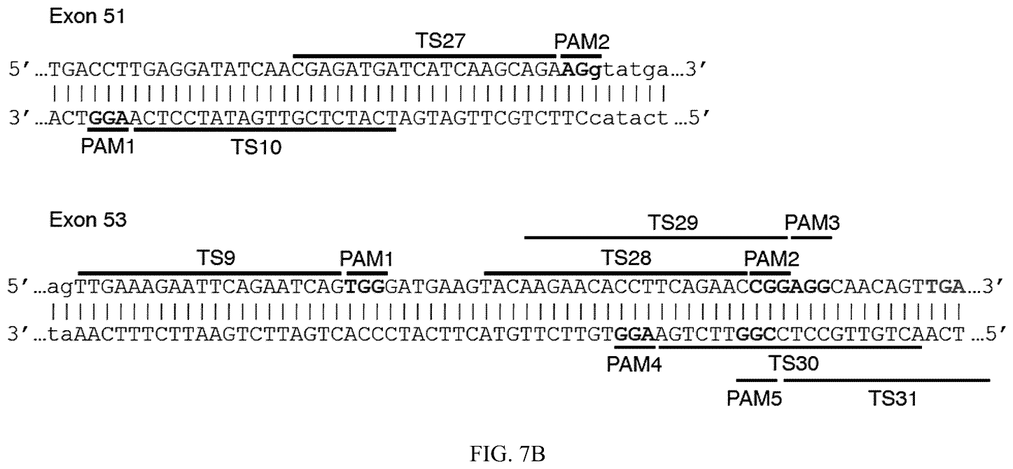

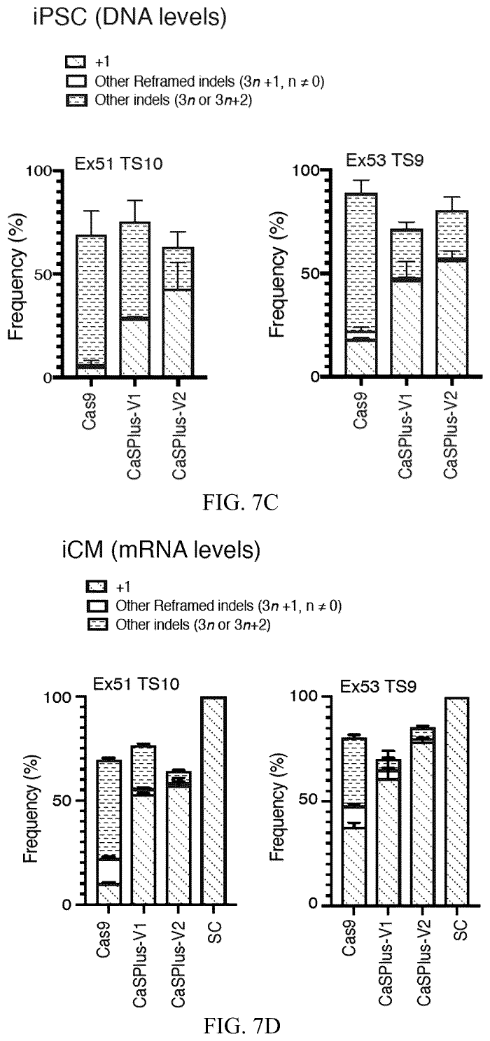

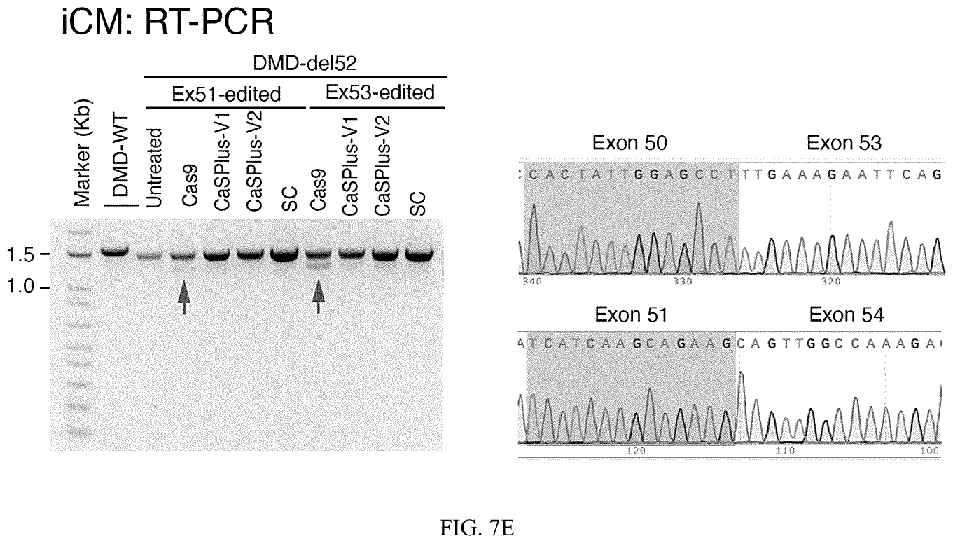

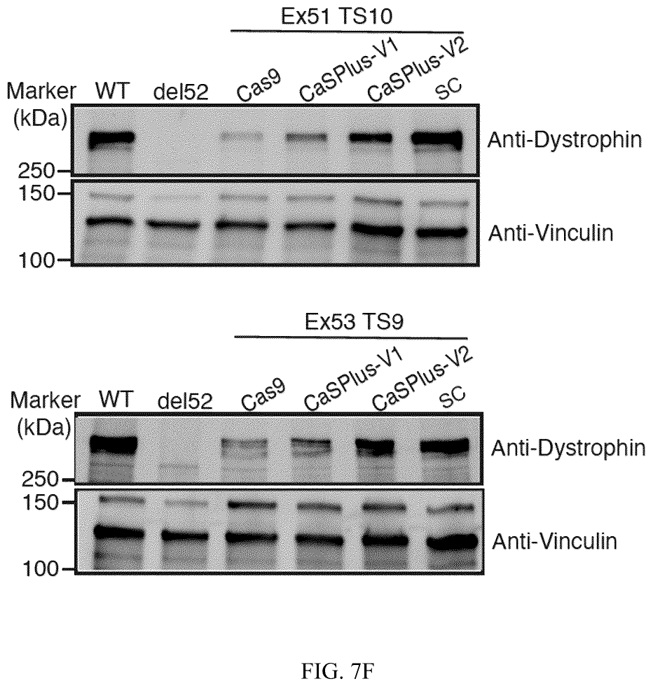

CasPlus system editing can enhance 1-bp insertions at the expense of small or large deletions at Cas9 target sites, making it a valuable tool for gene knock out and for the treatment of diseases caused by indels with 3n−1. Duchenne muscular dystrophy (DMD) is caused by out-of-frame mutations in the dystrophin gene, which lead to lethal degeneration of cardiac and skeletal muscle⁽³⁶⁾. Previously, we corrected DMD mutations via CRISPR/Cas9-mediated single-site editing on RNA splice sites or by double cutting to excise the exon^{(21, 37)}. Both strategies were designed to excise the exon to correct the open reading frame. However, single-site editing is limited to RNA splice sites, and double cutting may increase the risk of undesired large deletions, translocations, and other chromosomal rearrangements. With this in mind, we tested the efficacy of CasPlus-mediated single-site editing to correct DMD mutations. We initially generated an iPSC model of the DMD exon 52 deletion using CRISPR/Cas9 gene editing. We analyzed whether precise reinsertion of 1-bp at the 3′ end of exon 51 or 5′ end of exon 53, could efficiently repair the dystrophin gene in iPSCs with exon 52 deletion (FIG. 7A). We designed a comprehensive pool of guide RNAs containing NGG PAMs on for the two target regions (FIG. 7B) and tested their editing efficiency in HEK293T cells. We found that TS10 had a slightly higher editing efficiency than TS27. We also found that TS9 and TS28 exhibited a much higher editing efficiency than other guide RNAs targeting on exon 53. Therefore, we selected TS10 and TS9 to correct the DMD exon 52 deletion, in iPSCs. HTS revealed that CasPlus-V2 had the highest frequency of both 1-bp insertions and corrected reading frames in comparison to CasPlus-V1 or Cas9 alone (FIG. 7C). We further differentiated the pool of edited iPSCs and an iPSC single clone (SC) with 1-bp insertions into cardiomyocytes (iCMs). For each target site, we designed one set of RT-PCR primers to reveal the profile of small indels, and another to detect exon skipping caused by larger deletions. HTS results illustrated that the highest ratio of mRNA alleles with 1-bp insertions and corrected reading frames, was in CasPlus-V2 edited iCMs (FIG. 7D). We confirmed that large deletions occurred in cells edited with Cas9 alone, when targeting DMD exons 51 and 53 using TS9 and TS10 (FIGS. 6B-6E). We analyzed whether genes with large deletions lost part or all the target exon, thereby inducing target exon skipping on the mRNA levels. Sanger sequencing results confirmed that whole exon 51 and 53 skipping occurred in iCMs edited with Cas9 alone (FIG. 7E). Next, Western blot analysis revealed that dystrophin expression was restored in pools of edited iCMs. CasPlus-V1 and V2 treatment had higher dystrophin expression in comparison to Cas9 only control treatment. (FIG. 7F).

### Exogenous Template-Independent Correction of CFTR F508del Mutation Via Sequential CasPlus Editing.

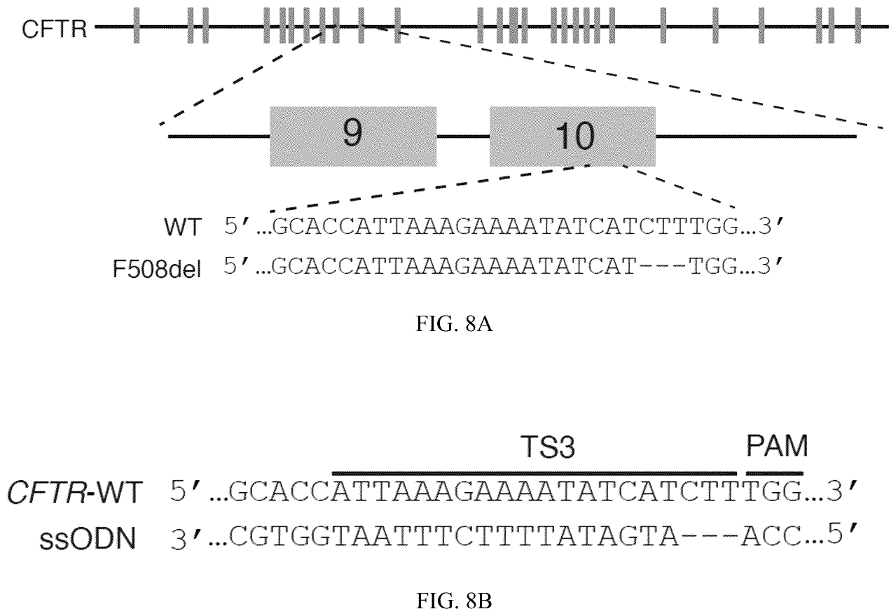

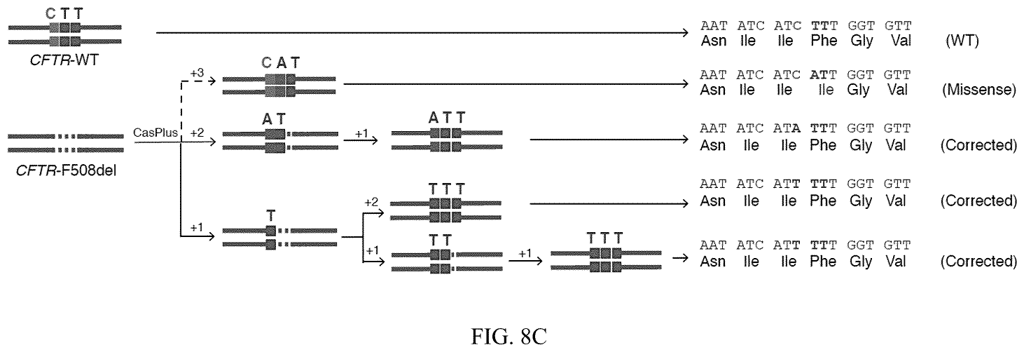

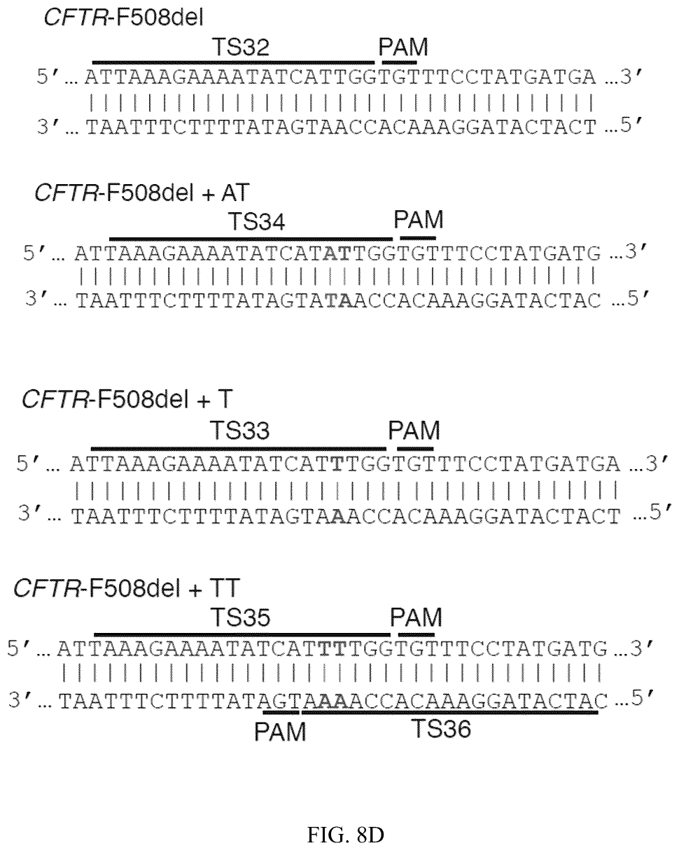

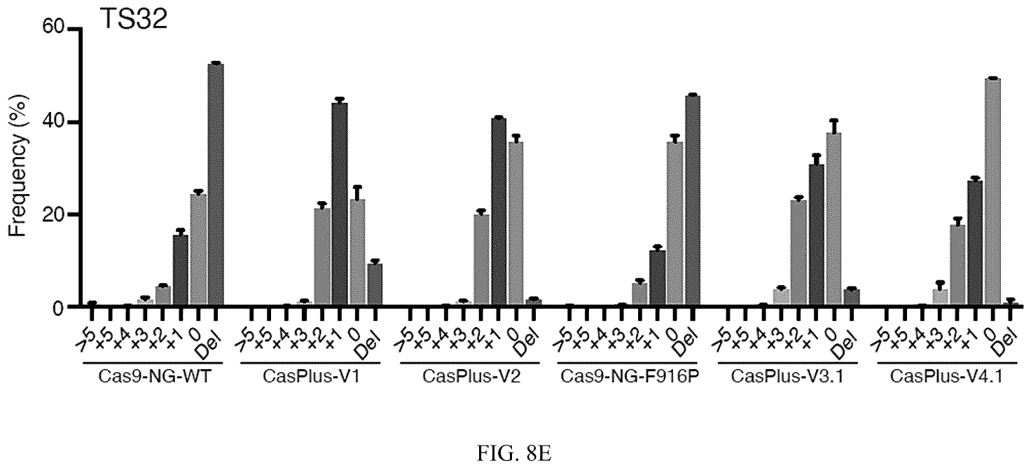

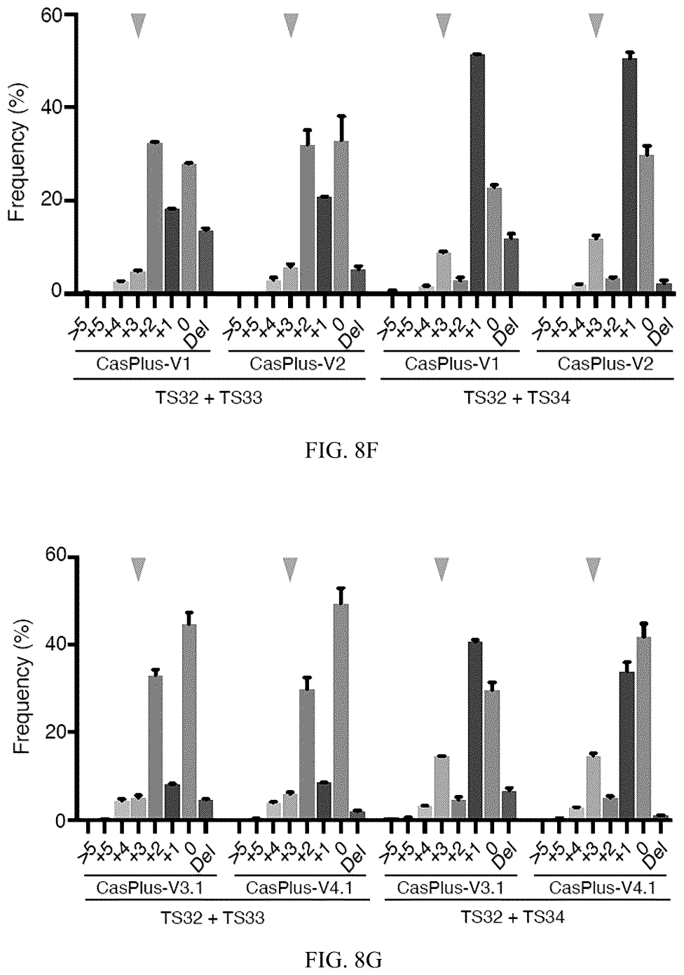

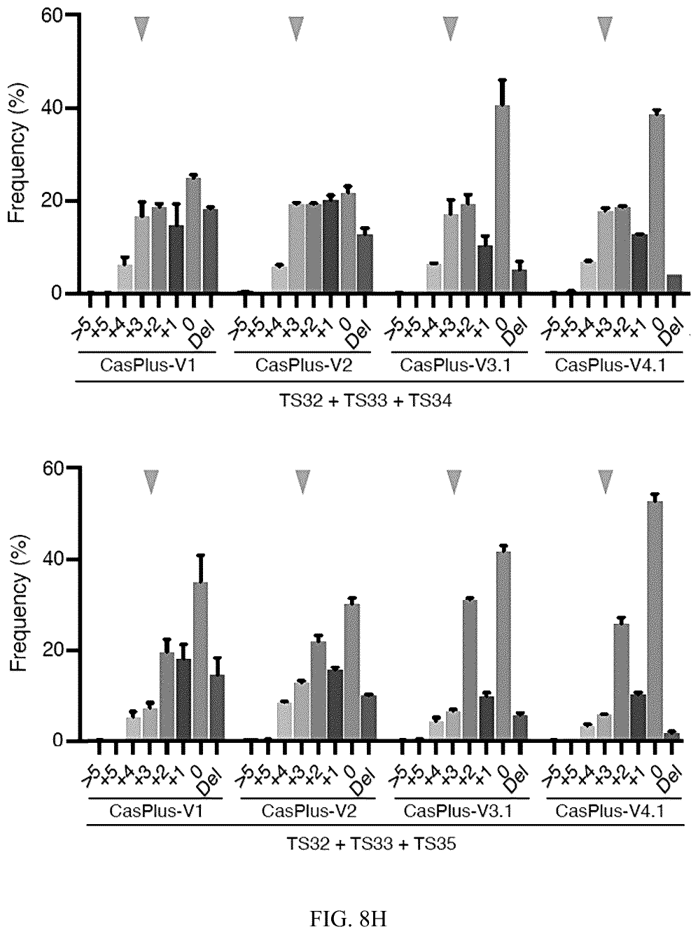

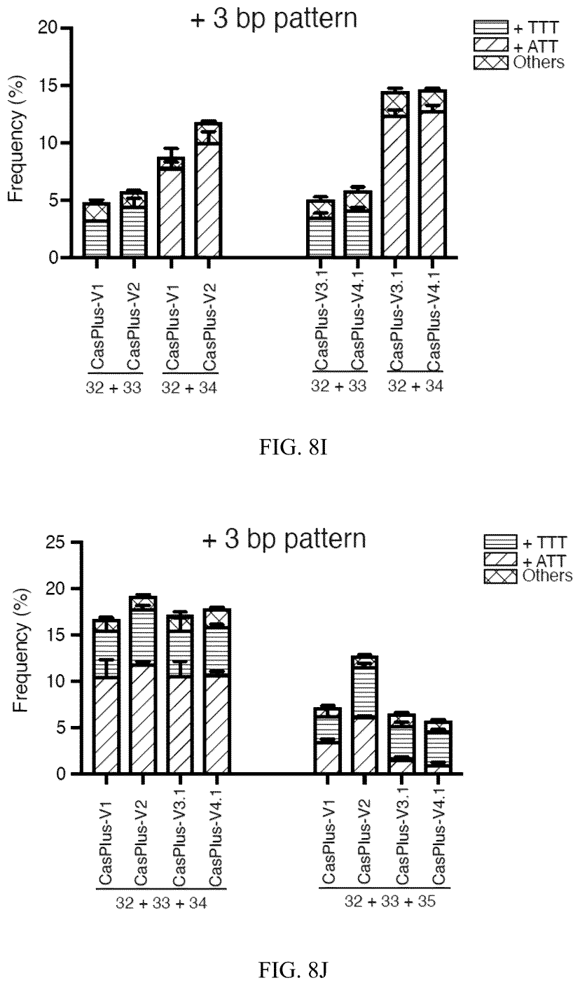

Exogenous template-independent insertions induced by CasPlus editing could be harnessed to precisely correct genetic diseases caused by 1 to 3-bp deletions. Cystic fibrosis is an autosomal recessive disease that involves functional defects in the mucus and sweat-producing cells, and severely affects multiple organs, especially the lungs. It is caused by mutations in the gene that produces the cystic fibrosis transmembrane conductance regulator (CFTR) protein^{(38, 39)} The most prevalent CFTR mutation is a 3-bp deletion that results in deletion of the phenylalanine located at position 508 (F508del), and accounts for approximately 70-80% of all pathogenic mutations in CFTR⁽⁴⁰⁾ (FIG. 8A). Drugs have been developed that improve clinical symptoms and prevent complications in CFTR patients⁽⁴¹⁾, however, the potential for genetic therapeutics that target the DNA level has barely been explored. Here, we employed sequential CasPlus editing to precisely correct the CFTR-F508del mutation. We initially generated a cellular model of CFTR-F508del in HEK293T cells using HDR-mediated knock-in (FIG. 8B). Based on the sequences flanking CFTR-F508del, we tested four potential outcomes of restoring gene expression via CasPlus editing: a CFTR protein with a missense amino acid (one-step editing), AT is inserted in the first step and T in the second step, T is inserted in the first step and TT in the second step, and the three-step incorporation of TTT, which would restore expression of the WT CFTR protein (FIG. 8C). We designed guide RNAs for sequential editing, initially targeting the CFTR-F508del allele (TS32), and then the intermediate AT insertion (TS34) or T, or containing a T (TS33) and/or TT (TS35 and TS36) to produce the desired edit (FIG. 8D). We first delivered vectors expressing guide RNA TS32 in combination with Cas9-NG-WT, Cas9-NG-F916P or CasPlus editors, into HEK293T cells with homozygous CFTR-F508del mutations. We observed that, with guide RNA (TS32), CasPlus-V1 and CasPlus-V2 or CasPlus-V3.1 and CasPlus-V4.1 had a higher frequency of 1 and 2-bp insertions relative to that with Cas9-NG-WT or Cas9-NG-F916P (FIG. 8E). Next, we tested two-step sequential CasPlus editing. We confirmed that CasPlus-V1, CasPlus-V2, CasPlus-V3.1 and CasPlus-V4.1 produced edits with 8%, 10%, 14.5% and 14.6% 3-bp insertions, respectively, with combinations of guide RNA (TS32) and (TS34). On the other hand, CasPlus-V1, CasPlus-V2, CasPlus-V3.1 and CasPlus-V4.1 generated edits with 3.3%, 4.5%, 5% and 6% 3-bp insertions, respectively, with the combination of guide RNA TS32 and TS33 (FIG. 8F-8G). We concluded that the combination of CasPlus-V3.1 or V4.1 with guide RNA TS32 and TS34 exhibited the highest percentage of 3-bp insertions. Additionally, cells treated with CasPlus-V3.1 or CasPlus-V4.1 with combinations of guide RNA TS32 and TS34 had editing profiles with approximately 30-40% of indels that were 1-bp insertions. Therefore, we analyzed whether the combination of guide RNAs TS32, TS33 and TS34 could further enhance the production of 3-bp insertions. We delivered CasPlus systems with guide RNA combination of TS32, TS33 and TS34 into homozygous CFTR-F508del cells, and confirmed that CasPlus-V1, V2, V3.1 and V4.2 induced 16%, 19%, 17% and 18% of edits with 3-bp insertions, respectively (FIG. 81I). We also tested three-step sequential CasPlus editing with guide RNAs TS32, TS34 and TS35. Results revealed that CasPlus-V2 exhibited the highest percentage of 3-bp insertions (12.8%). Analysis of the pattern of 3-bp insertions following sequential CasPlus editing, in combination with different guide RNAs, proved that >90% of 3-bp insertions are corrected CFTR edits with a silent mutation, rather than WT CFTR (FIGS. 8I-8J). Based on the results described above, we concluded that sequential CasPlus editing can efficiently and precisely correct CFTR-F508del mutations.

### Repression of On-Target Chromosomal Translocations Between Two Chromosomes by CasPlus Editing.

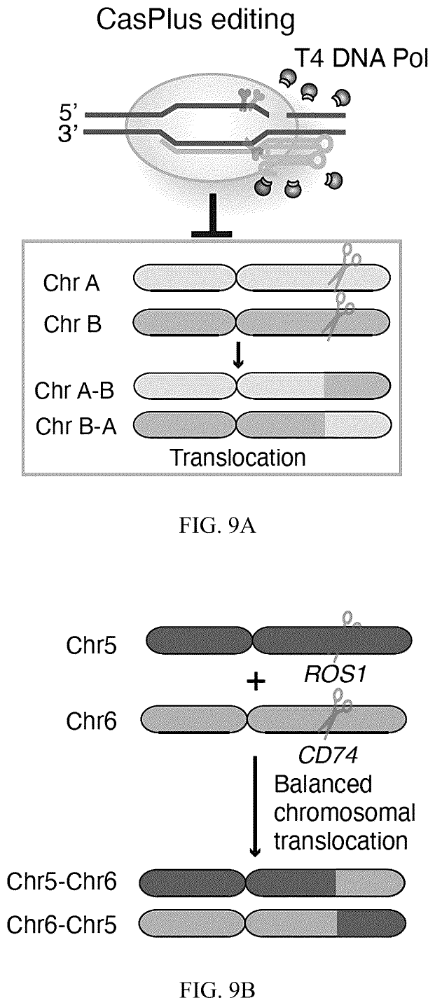

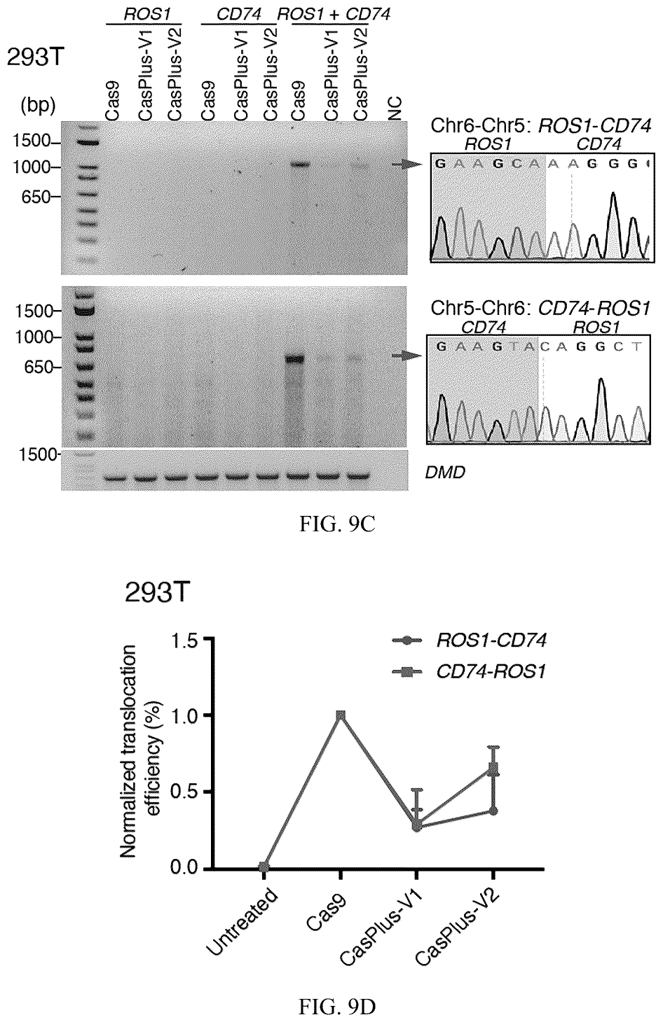

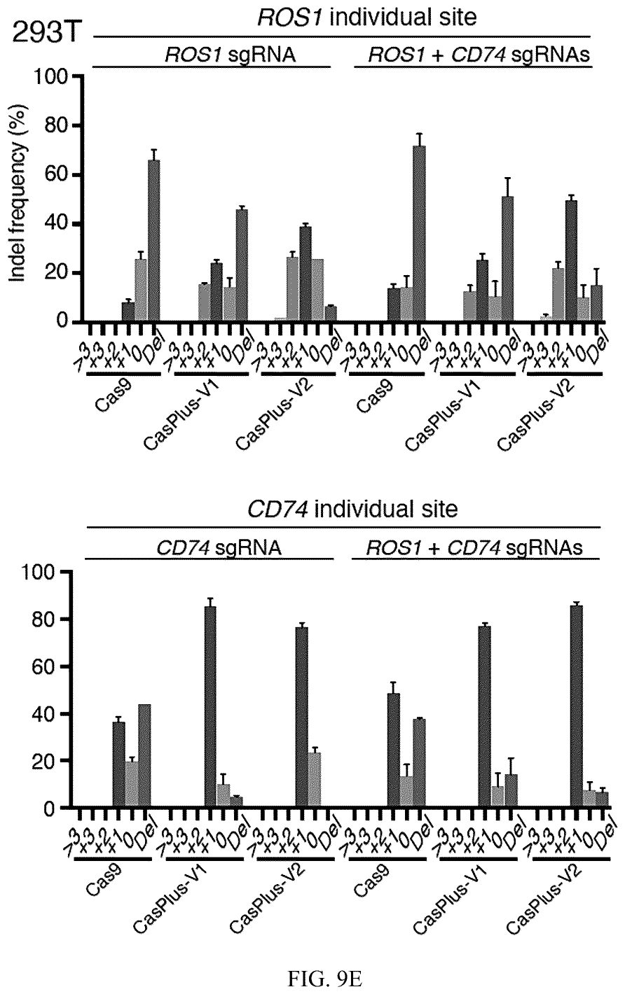

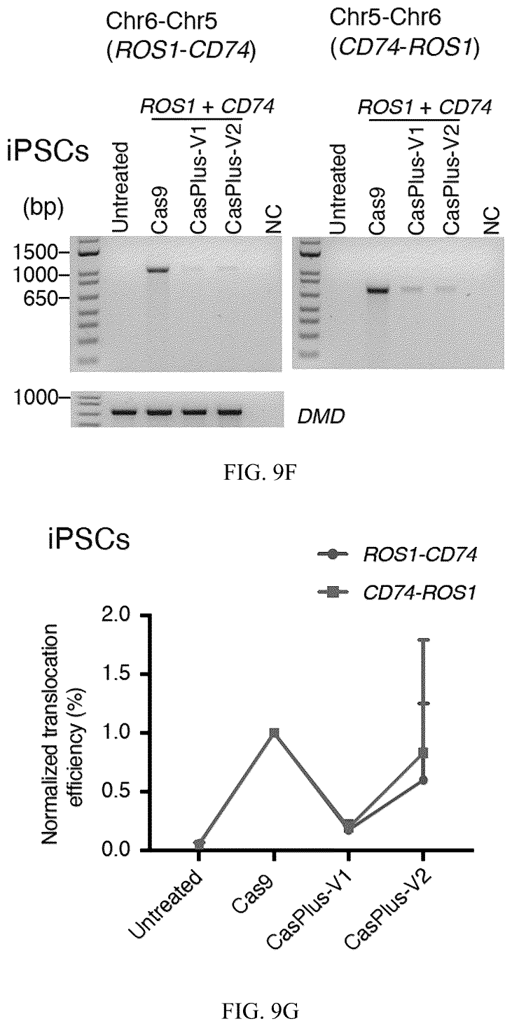

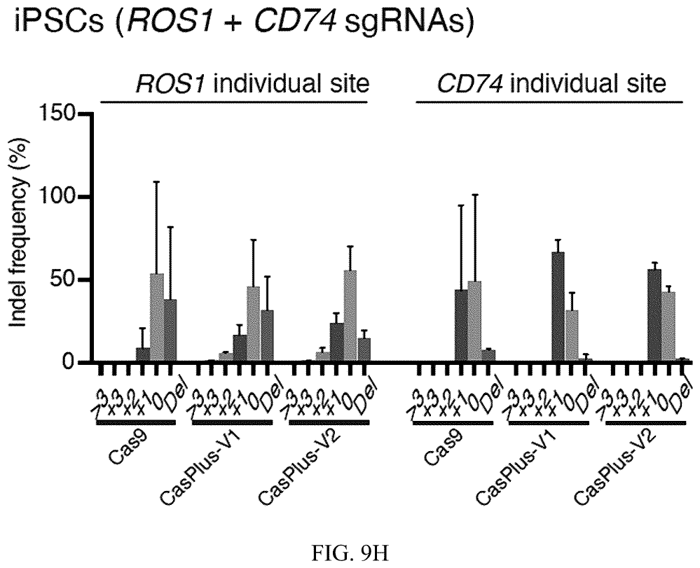

Chromosomal translocations occur when two simultaneous DSBs are present on two chromosomes (FIG. 9A). To investigate whether using CasPlus editing can reduce chromosomal translocations, we recapitulated previously described translocation events between the genes CD74 and ROS1 in HEK293T cells⁽⁴²⁾ (FIG. 9B). We PCR-amplified the breakpoint junction regions on the fused chromosomes and determined translocation efficiencies. We detected and verified both ROS1-CD74 and CD74-ROS1 translocations induced by Cas9 and CasPlus editing (FIG. 9C). The translocation frequencies were −5-fold lower with CasPlus-V1 and ˜2-fold lower with CasPlus-V2 compared to Cas9 editing (FIGS. 9C and 9D). The frequencies of insertions at ROS1 and CD74 individual sites were higher with CasPlus-V1 and -V2 editing compared to Cas9 editing (FIG. 9E). We observed similar trends of repression of chromosomal translocations in iPSCs (FIGS. 9F-91I).

### Repression of On-Target Chromosomal Translocations Among Multiple Chromosomes by CasPlus Editing.

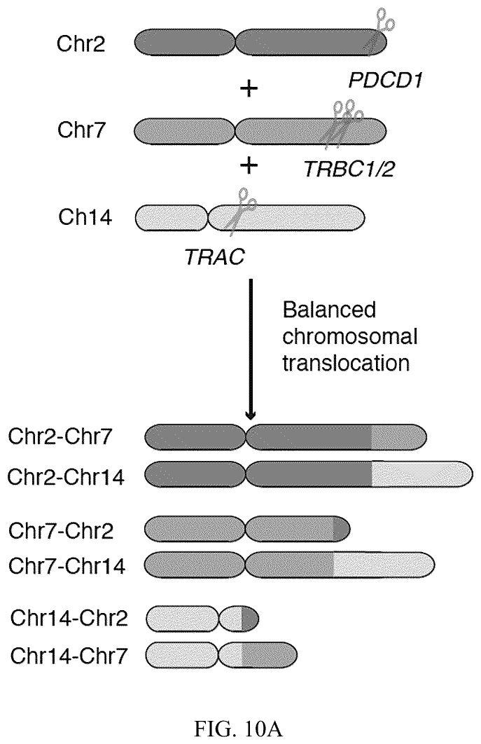

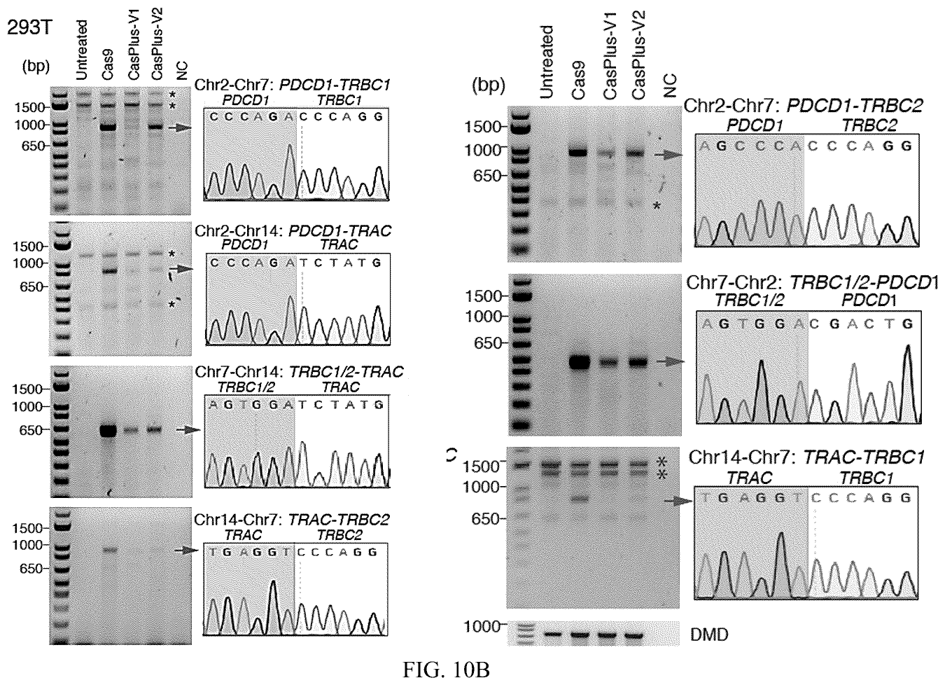

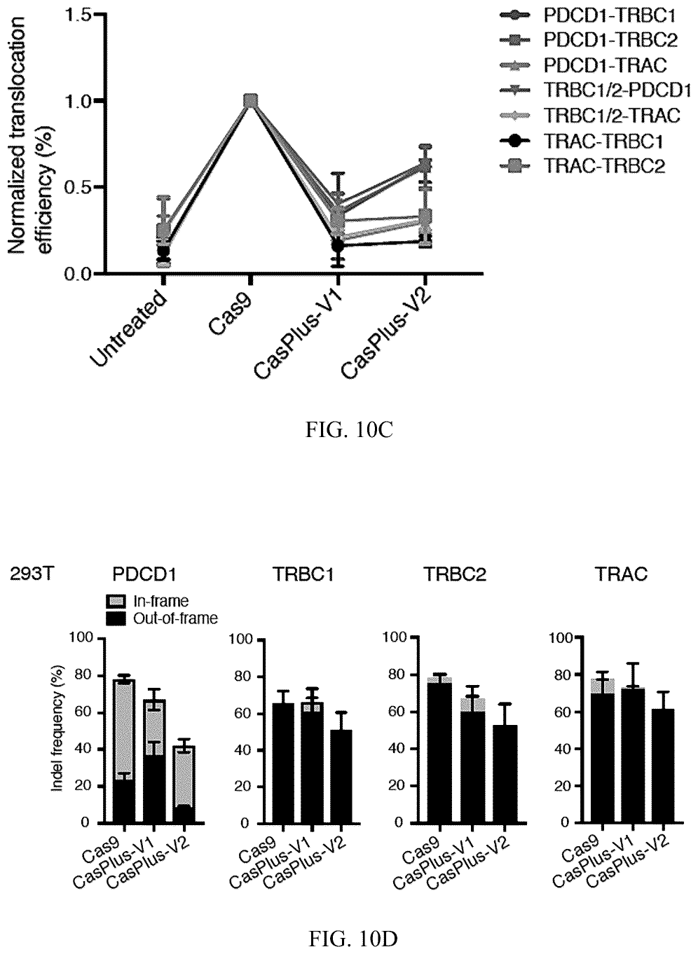

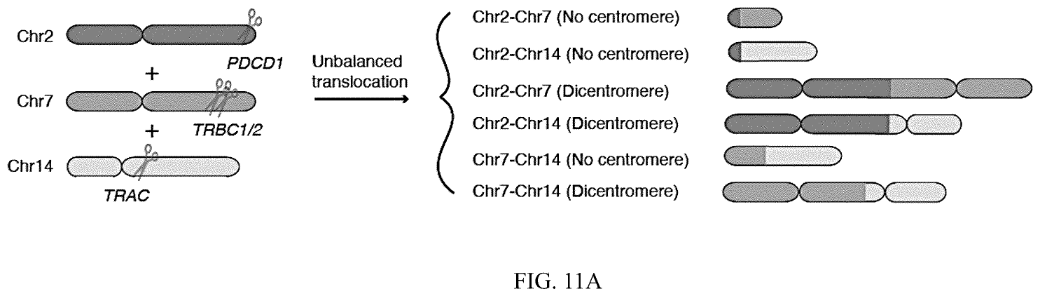

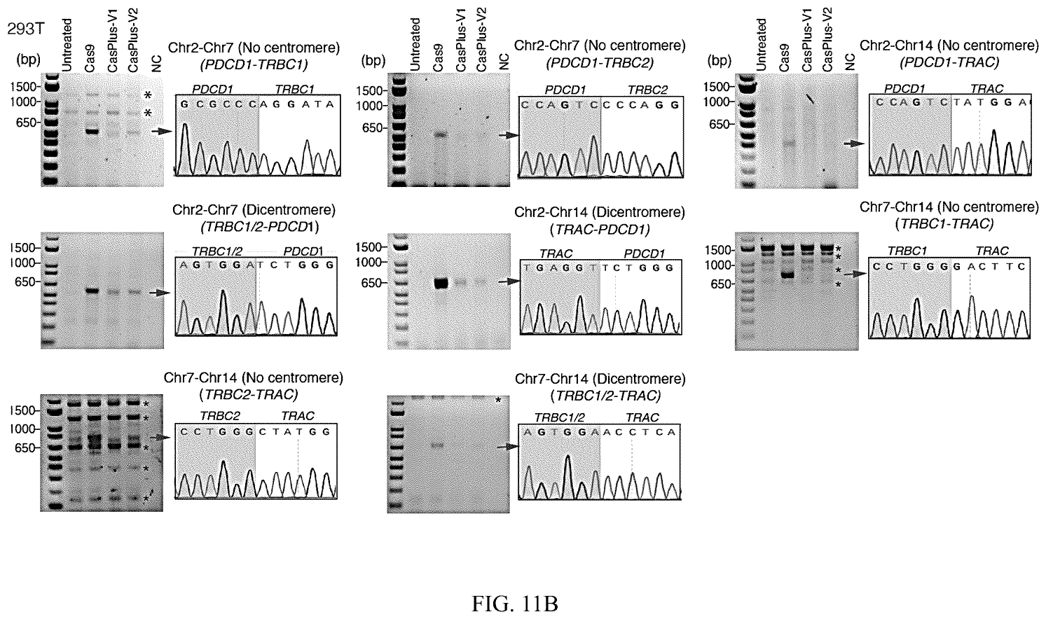

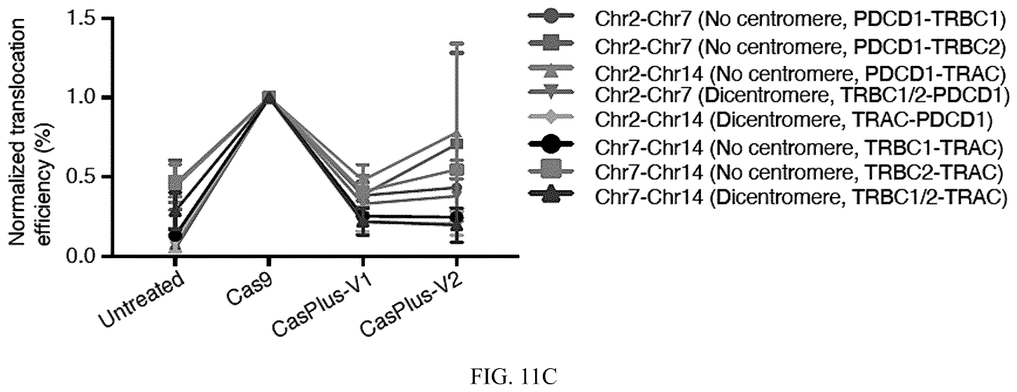

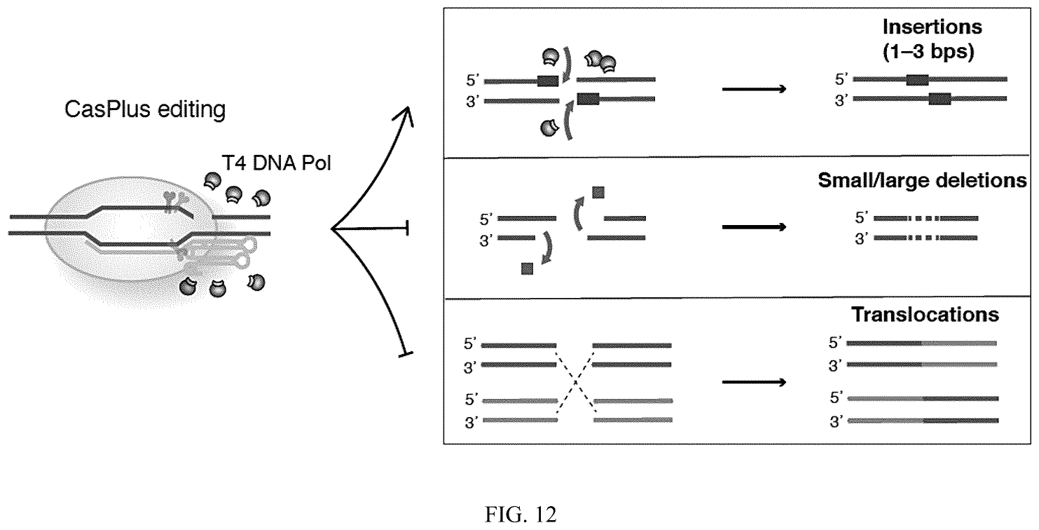

We next investigated the chromosomal translocations among the genes PDCD1, TRBC1, TRBC2, and TRAC (on chromosomes 2, 7, and 14) in HEK293T cells induced by the three gRNAs used in a previously T cell-based clinical trial^{(6, 7)} (FIG. 10A and FIG. 11A). CasPlus-V1 caused a 2.5-to-4.5-fold decrease in all types of translocations tested among these four genes (FIGS. 10B and 10C and FIGS. 11B and 11C). CasPlus-V1 editing induced a comparable knockout efficiency at these four individual sites when compared to Cas9 editing (FIG. 10D). CasPlus-V2 had a similar knockout effect to CasPlus-V1 but was less efficient in repressing translocations. Our proof-of-concept results thus indicate that CasPlus editing significantly represses Cas9-mediated on-target chromosomal translocations and is a potentially safer approach for T cell-relevant therapy.

### REFERENCES—THIS REFERENCE LISTING IS NOT AN INDICATION THAT ANY REFERENCE IS MATERIAL TO PATENTABILITY

1. M. Jinek et al., A programmable dual-RNA-guided DNA endonuclease in adaptive bacterial immunity. Science 337, 816-821 (2012).
2. M. Jinek et al., RNA-programmed genome editing in human cells. Elife 2, e00471 (2013).
3. L. Cong et al., Multiplex genome engineering using CRISPR/Cas systems. Science 339, 819-823 (2013).
4. P. Mali et al., RNA-guided human genome engineering via Cas9. Science 339, 823-826 (2013).
5. M. Kosicki, K. Tomberg, A. Bradley, Repair of double-strand breaks induced by CRISPR-Cas9 leads to large deletions and complex rearrangements. Nat Biotechnol 36, 765-771 (2018).
6. A. D. Nahmad et al., Frequent aneuploidy in primary human T cells after CRISPR-Cas9 cleavage. Nat Biotechnol, (2022).
7. E. A. Stadtmauer et al., CRISPR-engineered T cells in patients with refractory cancer. Science 367, (2020).
8. M. L. Leibowitz et al., Chromothripsis as an on-target consequence of CRISPR-Cas9 genome editing. Nat Genet 53, 895-905 (2021).
9. F. Uddin, C. M. Rudin, T. Sen, CRISPR Gene Therapy: Applications, Limitations, and Implications for the Future. Front Oncol 10, 1387 (2020).
10. X. Shi et al., Cas9 has no exonuclease activity resulting in staggered cleavage with overhangs and predictable di- and tri-nucleotide CRISPR insertions without template donor. Cell Discov 5, 53 (2019).
11. H. H. Y. Chang, N. R. Pannunzio, N. Adachi, M. R. Lieber, Non-homologous DNA end joining and alternative pathways to double-strand break repair. Nat Rev Mol Cell Biol 18, 495-506 (2017).
12. D. D. G. Owens et al., Microhomologies are prevalent at Cas9-induced larger deletions. Nucleic Acids Res 47, 7402-7417 (2019).
13. M. Kosicki et al., Cas9-induced large deletions and small indels are controlled in a convergent fashion. Nat Commun 13, 3422 (2022).
14. M. W. Shen et al., Predictable and precise template-free CRISPR editing of pathogenic variants. Nature 563, 646-651 (2018).
15. F. Allen et al., Predicting the mutations generated by repair of Cas9-induced double-strand breaks. Nat Biotechnol, (2018).
16. R. T. Leenay et al., Large dataset enables prediction of repair after CRISPR-Cas9 editing in primary T cells. Nat Biotechnol 37, 1034-1037 (2019).
17. A. M. Chakrabarti et al., Target-Specific Precision of CRISPR-Mediated Genome Editing. Mol Cell 73, 699-713 e696 (2019).
18. K. F. O'Brien, L. M. Kunkel, Dystrophin and muscular dystrophy: past, present, and future. Mol Genet Metab 74, 75-88 (2001).
19. F. Muntoni, S. Torelli, A. Ferlini, Dystrophin and mutations: one gene, several proteins, multiple phenotypes. Lancet Neurol 2, 731-740 (2003).
20. R. Adorisio et al., Duchenne Dilated Cardiomyopathy: Cardiac Management from Prevention to Advanced Cardiovascular Therapies. J Clin Med 9, (2020).
21. C. Long et al., Correction of diverse muscular dystrophy mutations in human engineered heart muscle by single-site genome editing. Sci Adv 4, eaap9004 (2018).
22. C. Long et al., Postnatal genome editing partially restores dystrophin expression in a mouse model of muscular dystrophy. Science 351, 400-403 (2016).
23. C. Long et al., Prevention of muscular dystrophy in mice by CRISPR/Cas9-mediated editing of germline DNA. Science 345, 1184-1188 (2014).
24. L. J. Reha-Krantz, Amino acid changes coded by bacteriophage T4 DNA polymerase mutator mutants. Relating structure to function. J Mot Biol 202, 711-724 (1988).
25. L. J. Reha-Krantz, Regulation of DNA polymerase exonucleolytic proofreading activity: studies of bacteriophage T4 “antimutator” DNA polymerases. Genetics 148, 1551-1557 (1998).
26. A. K. Abdus Sattar, T. C. Lin, C. Jones, W. H. Konigsberg, Functional consequences and exonuclease kinetic parameters of point mutations in bacteriophage T4 DNA polymerase. Biochemistry 35, 16621-16629 (1996).
27. H. K. Dressman, C. C. Wang, J. D. Karam, J. W. Drake, Retention of replication fidelity by a DNA polymerase functioning in a distantly related environment. Proc Natl Acad Sci USA 94, 8042-8046 (1997).
28. K. Hori, D. F. Mark, C. C. Richardson, Deoxyribonucleic acid polymerase of bacteriophage T7. Characterization of the exonuclease activities of the gene 5 protein and the reconstituted polymerase. J Biol Chem 254, 11598-11604 (1979).
29. T. L. Capson et al., Kinetic characterization of the polymerase and exonuclease activities of the gene 43 protein of bacteriophage T4. Biochemistry 31, 10984-10994 (1992).
30. B. Zetsche et al., Cpf1 is a single RNA-guided endonuclease of a class 2 CRISPR-Cas system. Cell 163, 759-771 (2015).
31. D. Kim et al., Genome-wide analysis reveals specificities of Cpf1 endonucleases in human cells. Nat Biotechnol 34, 863-868 (2016).
32. M. Hogg, W. Cooper, L. Reha-Krantz, S. S. Wallace, Kinetics of error generation in homologous B-family DNA polymerases. Nucleic Acids Res 34, 2528-2535 (2006).
33. J. Shou, J. Li, Y. Liu, Q. Wu, Precise and Predictable CRISPR Chromosomal Rearrangements Reveal Principles of Cas9-Mediated Nucleotide Insertion. Mol Cell 71, 498-509 e494 (2018).
34. H. Y. Shin et al., CRISPR/Cas9 targeting events cause complex deletions and insertions at 17 sites in the mouse genome. Nat Commun 8, 15464 (2017).
35. B. Farboud, A. F. Severson, B. J. Meyer, Strategies for Efficient Genome Editing Using CRISPR-Cas9. Genetics 211, 431-457 (2019).
36. K. P. Campbell, S. D. Kahl, Association of dystrophin and an integral membrane glycoprotein. Nature 338, 259-262 (1989).
37. Y. Zhang et al., CRISPR-Cpf1 correction of muscular dystrophy mutations in human cardiomyocytes and mice. Sci Adv 3, e1602814 (2017).
38. B. P. O'Sullivan, S. D. Freedman, Cystic fibrosis. Lancet 373, 1891-1904 (2009).
39. S. D. Patel, T. R. Bono, S. M. Rowe, G. M. Solomon, CFTR targeted therapies: recent advances in cystic fibrosis and possibilities in other diseases of the airways. Eur Respir Rev 29, (2020).
40. P. B. Davis, Cystic fibrosis since 1938. Am J Respir Crit Care Med 173, 475-482 (2006).
41. M. M. Rafeeq, H. A. S. Murad, Cystic fibrosis: current therapeutic targets and future approaches. J Transl Med 15, 84 (2017).
42. P. S. Choi, M. Meyerson, Targeted genomic rearrangements using CRISPR/Cas technology. Nat Commun 5, 3728 (2014).
43. F. A. Ran et al., Genome engineering using the CRISPR-Cas9 system. Nat Protoc 8, 2281-2308 (2013).
44. L. Pinello et al., Analyzing CRISPR genome-editing experiments with CRISPResso. Nat Biotechnol 34, 695-697 (2016).
45. Statistical Genomics. Methods and Protocols. Anticancer Res 36, 3224 (2016).
46. H. Li, Minimap2: pairwise alignment for nucleotide sequences. Bioinformatics 34, 3094-3100 (2018).

### Materials and Methods

### Plasmids

The vector pSpCas9(BB)-2A-GFP (PX458) (Addgene plasmid #48138) containing the human-codon-optimized SpCas9 gene with 2A-GFP and the sgRNA backbone was purchased from Addgene. pLentiV-SgRNA-tdTomato-P2A-BlasR (Addgene plasmid #110854) and EF1A-CasRx-2A-EGFP (Addgene Plasmid #109049) were gifts from Dr. Lukas Dow and Dr. Patrick Hsu, respectively. To construct the lentiviral vector expressing tdTomato-d151A, the tdTomato-d151A gene was synthesized by Integrated DNA Technologies (IDT). First, it was cloned into vector p3×Flag-CMV-10, then the CMV-10-tdtomato-d151A was cloned into pLentiv-SgRNA-tdTomato-P2A-BlasR using MluI and BamHI restriction sites. For DNA polymerase cloning, the coding sequences of DNA polymerase 4, DNA polymerase I, Klenow fragment, T4 DNA polymerase, RB69 DNA polymerase, and T7 DNA polymerase were codon-optimized for human cell expression using the Genewiz Codon Optimization tool. For each DNA polymerase, an expression cassette containing the polymerase, an MS2 (MS2 bacteriophage coat protein) and a hemagglutinin (HA) tag, two copies of a nuclear localization sequence (NLS), and a flexible linker was synthesized from Genewiz and cloned into EF1A-CasRx-2A-EGFP via Gibson assembly. Mutations of T4 DNA polymerase and RB69 DNA polymerase were introduced into the vectors EF1A-MS2-T4-DNA-Polymerase-2A-EGFP and EF1A-MS2-RB69-DNA-polymerase-2A-EGFP, respectively, via Gibson assembly. Mutations of Cas9 were generated in the backbone pSpCas9(BB)-2A-GFP (PX458) via Gibson assembly. Guide RNA cloning was carried out according to the CRIPSR plasmid instructions from the Feng Zhang Lab(43). All guide RNA sequences are listed in Table 1. All sequences synthesized for either tdTomato-d151A or DNA polymerase clones are listed in Table 3.

### Cell Lines

Generation of a HEK293T cell line containing the tdTomato-d151A reporter. To generate a stable tdTomato-d151A reporter cell line in HEK293T cells, we co-transfected pLentiV vector expressing tdTomato-d151A and the lentiviral helper plasmids psPAX2, pMD2G, and pEGFP into HEK293T cells. Single cells expressing GFP were isolated in 96-well plates 72 h post-transfection and genotyped 2 weeks later. Positive clones were then stored and expanded for subsequent experiments.

Generation of HEK293T cells containing homozygous CFTR-F508del mutations. HEK293T cell lines containing homozygous CFTR-F508del mutations were generated via HDR-mediated gene editing. The DNA template for CFTR-F508del knock-in was synthesized by IDT. To generate the mutant HEK293T cell line, the DNA template was co-transfected with a vector expressing Cas9, GFP, and TS3. Single cells expressing GFP were isolated in 96-well plates 72 h post-transfection and genotyped 2 weeks later. Positive clones containing the homozygous CFTR-F508del mutation were stored and expanded for subsequent experiments. The template for knock-in is shown in table 3. The sequence of TS3 is shown in Table 1.

Generation of male iPS cells containing the DMD exon 52 deletion. Male iPSCs were electroporated with vectors expressing Cas9, GFP, and a pair of guide RNAs specific for the deletion (DMD-Ex52-g1 and DMD-Ex52-g2, see Table 1). Single cells expressing GFP were isolated in 96-well plates 72 h post-transfection and genotyped 2 weeks later. Positive clones containing the DMD exon 52 deletion were stored and expanded for subsequent experiments.

Sample Preparation, DNA Isolation and PCR Amplicon Preparation for Deep Sequencing

Transfection and sorting of HEK293T cells. HEK293T cells were transfected using Lipofectamine 2000 Transfection Reagent (ThermoFisher LifeTech) according to the manufacturer's instructions. Cell sorting was performed by the Flow Cytometry Core Facility at New York University Grossman Medical Center 72 h post-transfection. Briefly, HEK293T cells were co-transfected with vectors expressing Cas9, a sgRNA targeting different genomic site, GFP and one of the DNA polymerases. Seventy-two hours post-transfection, transfected cells were dissociated using a trypsin-EDTA solution (Corning) for 2 min at 37° C. Subsequently, 2 ml of warm Dulbecco's modified Eagle's medium (DMEM) (Corning) supplemented with 10% fetal bovine serum (FBS) (Gemini Bio-Products) was added. The resuspended cells were transferred into a 15-ml Falcon tube and centrifuged at 1000 rpm for 5 min at room temperature. The medium was then removed, and the cells resuspended in 0.4-1 ml DMEM. Cells were filtered through the 50-μm-mesh cap of a CellTrix strainer (Sysmex). Cells expressing GFP were sorted by flow cytometry into a 5-ml polypropylene round-bottom Tube (Corning) for immediate DNA extraction.

Isolation of raw DNA from sorted cells. Protease K (20 mg/ml) was added to DirectPCR Lysis Reagent (Viagen Biotech Inc.) to a final concentration of 1 mg/ml. Sorted cells (4×10⁴-1×10⁵) were centrifuged at 4° C. at 12000 rpm for 5 min and the supernatant discarded. Cell pellets were resuspended in 20-50 μL of DirectPCR/protease K solution, incubated at 55° C. for >2 hours or until no clumps were observed, incubated at 85° C. for 30 min, and then spin down briefly (10 sec). 1-2 μL DNA was used for PCR amplification. All PCR primer sequences are described herein.

PCR amplicon preparation for deep sequencing. To prepare for deep sequencing, PCR amplicons of −300 bp were amplified using a GoTaq kit (Promega), separated on a 2% agarose gel, and purified with the MinElute Gel Extraction Kit (Qiagen). For each sample, 100 ng of gel-purified PCR product was barcoded with the Nextera Flex Prep HT kit according to the manufacturer's instructions and sequenced using the MiSeq paired-end 150-cycle format by the Genome Technology Center Core Facility at New York University Grossman Medical Center.

Detection of large deletions. Male DMD-del52 iPSCs were electroporated with vectors expressing Cas9, GFP, and the guide RNA G10 or G9 either alone or in combination with either T4-WT or T4-D219A. Electrorated cells were then sorted into GFP⁺ populations 72 hr post-electroporation. Sorted cells were expanded. DNA was isolated from expanded cells 2 weeks later and subjected to large deletions detection. Single cells were isolated from edited cell pools into 96-well plates 2 weeks after electroporation and genotyped 2 weeks later. Single cells containing one insert of G at DMD exon 51 or T at DMD exon 53 were stored and expanded for subsequent experiments. Edited iPSCs and the single clones containing 1-bp insertion were further differentiated into iCMs. DNA was isolated from iCMs and subjected to large deletions detection.

Detection of chromosomal translocations. HEK293T cells were co-transfected with vectors expressing Cas9, GFP, and guide RNAs targeting either ROS1 and CD74 or PDCD1, TRAC, and TRBC1/TRBC2 either alone or in combination with T4-WT or T4-D219A. Transfected cells were sorted into GFP⁺ populations 72 hr after transfection and sorted cells (1×10⁶) were immediately subjected to DNA extraction. Chromosomal translocations were detected by PCR using primers specifically recognizing the breakpoint junction region of each fused chromosomes. All the guide RNAs used were summarized in Table 1.

Human iPSC maintenance and nucleofection. Human iPSC lines were cultured in Stemflex™ medium (ThermoFisher) and passaged approximately every 3 days (1:8-1:12 split ratio). One hour before nucleofection, iPSCs were treated with 10 μM ROCK inhibitor (Y-27632) and dissociated into single cells using Accutase (Innovative Cell Technologies Inc.). Cells (8×10⁵) were mixed with 2 μg of a vector expressing Cas9, GFP, and guide RNA, as well as 2 μg of a vector encoding a DNA polymerase. This mixture was electroporated into cells using the P3 Primary Cell 4D-Nucleofector X kit (Lonza) according to the manufacturer's protocol. After nucleofection, iPSCs were cultured in StemFlex medium supplemented with CloneR (10×) (StemCell Technologies) and antibiotic-antimycotic (100×) (ThermoFisher). Three days after nucleofection, cells expressing GFP were sorted as described above and replated in StemFlex medium. Ten to fifteen days after sorting, cells were harvested for DNA isolation.

Cardiomyocyte differentiation and purification. Human iPSCs (edited iPSC pools or single clones with 1-bp insertions) were induced for differentiation into cardiomyocytes according to the manufacturer's instructions using the PSC Cardiomyocyte Differentiation Kit (ThermoFisher Scientific). At 15-20 days after differentiation initiation, cells were purified in RPMI-1640 medium lacking glucose supplemented with B27 (ThermoFisher Scientific). Cells were cultured in this medium for 2-4 days. Cardiomyocytes were used for experiments on day 40-50 after the initiation of differentiation.

RNA extraction and cDNA synthesis. RNA from iPSC-derived cardiomyocytes was extracted using TRIzol (catalog 15596026; Thermo Fisher Scientific) according to the manufacturer's protocol. cDNA was synthesized using the Superscript III First-Strand cDNA Synthesis Kit (ThermoFisher LifeTech) according to the manufacturer's instructions. All RT-PCR primer sequences are described herein.

Western blotting. HEK293T cells and cardiomyocytes (iCMs) differentiated from iPSCs were harvested, centrifuged, and lysed with RIPA lysis buffer (Santa Cruz Biotechnology) according to the manufacturer's protocol. Samples were lysed and centrifuged, and the supernatant was incubated at 95° C. for 10 minutes in the presence of Laemmli sample buffer (catalog 161-0747; Bio-Rad). Proteins (20 μg per sample) were separated on Mini-PROTEAN TGX 4-15% precast SDS-PAGE gels (Bio-Rad) for 1-2 h at 120 V and then transferred to PVDF membrane at 250 mA for 1-4 h. Membranes were probed overnight at 4° C. either with anti-HA antibody (catalog no. M180-3; MBL) and anti-glyceraldehyde-3-phosphate dehydrogenase antibody (catalog no. MAB374; Sigma) or with anti-dystrophin (catalog no. ab7817; abcam) and anti-vinculin antibody (catalog no. V9131; Sigma-Aldrich). Membranes were then washed, probed with a goat anti-mouse or goat anti-rabbit IgG H+L-HRP conjugated secondary antibody (1:10000) (Bio-Rad) for 1 h, and visualized by western blot with Luminol reagent (Santa Cruz) according to the manufacturer's protocol.

PCR amplicon preparation for PacBio sequencing. To prepare samples for PacBio sequencing, genomic DNA was extracted from iPSCs using the DNeasy Blood and Tissue Kit. Barcodes were added to the target region via a two-step PCR reaction. The first-round PCR was performed using LA Taq DNA polymerase (Takara) according to the manufacturer's instructions. The first round amplified a 5-kb region around the target site using target-specific primers tailed with universal forward and reverse sequences. The second round of PCR re-amplified and barcoded the first round of PCR products using universal, barcoded forward and reverse primers. The final barcoded PCR products were sequenced using the SMRTCell (1M v3 LR) platform by the Genome Technology Center Core Facility at New York University Grossman Medical Center.

### Bioinformatic Analysis

Deep sequencing. To detect indels in the deep sequencing data, unmapped paired-end amplicon deep sequencing reads were used as inputs into the CRISPResso2 tool to quantify the frequency of editing events⁽⁴⁴⁾. The tool was run with default parameters (https://github.com/pinellolab/CRISPResso2).

PacBio sequencing. Raw PacBio data were demultiplexed with the corresponding barcode using the SMRTlink software to assign barcoded reads to each sample (smrtlink version: 8.0.0.80529, chemistry bundle: 8.0.0.778409, params: 8.0.0). Analysis of demultiplexed data was performed using PacBio tools distributed via Bioconda (https://github.com/PacificBiosciences/pbbioconda). For DMD exon 51 and 53 locus pileup, circular consensus sequences were converted to HiFi calls using the pbccs command and filtering for reads with support from at least three full-length subreads. The resulting fastq files were used as inputs to a custom python script that filtered for reads containing specific 50-bp index sequences at both the 5′ and 3′ regions of each read. Resulting filtered reads were mapped to the reference genome using minimap2 (ax splice --splice-flank=no -u no -G 5000). The genome coverage of the alignment files was calculated using the “bedtools genomecov -d” (v 2.27.1) command with all downstream analyses performed using custom R script (v4.1.1) and visualized with the Gvizl package^{(45, 46)}. For DMD exon 51, the 5′ index sequence is tttttccaaacgtgcttttcaggaaacagtggtctgcttgttgaagtctg (SEQ ID NO: 60), and the 3′ index sequence is aatcctggaccagaggttccattgagctgagatcacaccattgcactcca (SEQ ID NO: 61). For DMD exon 53, the 5′ index sequence is ggactatatttttgatttcatgttacaatcactagttttgtggggtcttt (SEQ ID NO: 62), and the 3′ index sequence is tgatgtgtattgctgcagattcaatgtaagttcccgatacagataaagat (SEQ ID NO: 63).

TABLE 1

| Target | Target | | Sequence |
|---|---|---|---|
| site | gene | Guide RNA | Identifier |
| TS2 | DHPS | UCCAGGAACAGCUGGGUACC | SEQ ID NO: 64 |
| TS3 | CFTR | AUUAAAGAAAAUAUCAUCUU | SEQ ID NO: 65 |
| TS5 | DMD | ACCUUCACUGGCUGAGUGGC | SEQ ID NO: 66 |
| TS9 | DMD | UUGAAAGAAUUCAGAAUCAG | SEQ ID NO: 67 |
| TS10 | DMD | UCAUCUCGUUGAUAUCCUCA | SEQ ID NO: 68 |
| TS11 | DMD | UCCUACUCAGACUGUUACUC | SEQ ID NO: 69 |
| TS12 | LMNA | GGGGCCAGGUGGCCAAGGUG | SEQ ID NO: 70 |
| TS17 | DMD | UAUGUGUUACCUACCCUUGU | SEQ ID NO: 71 |
| TS18 | DMD | GGUUGCUUCAUUACCUUCAC | SEQ ID NO: 72 |
| TS19 | HEXA | UACCUGAACCGUAUAUCCUA | SEQ ID NO: 73 |
| TS22 | DMD | UCCAGGAUGGCAUUGGGCAG | SEQ ID NO: 74 |
| TS24 | DMD | ACCAGAGUAACAGUCUGAGU | SEQ ID NO: 75 |
| TS25 | DMD | UAUAAAAUCACAGAGGGUGA | SEQ ID NO: 76 |
| TS26 | LMNA | CCUGCAGGGUGGCCUCACCU | SEQ ID NO: 77 |
| TS27 | DMD | CGAGAUGAUCAUCAAGCAGA | SEQ ID NO: 78 |
| TS28 | DMD | UACAAGAACACCUUCAGAAC | SEQ ID NO: 79 |
| TS29 | DMD | AAGAACACCUUCAGAACCGG | SEQ ID NO: 80 |
| TS30 | DMD | ACUGUUGCCUCCGGUUCUGA | SEQ ID NO: 81 |
| TS31 | DMD | UUUCAUUCAACUGUUGCCUC | SEQ ID NO: 82 |
| TS32 | CFTR- | AUUAAAGAAAAUAUCAUUGG | SEQ ID NO: 83 |
| | F508del | | |
| TS33 | CFTR- | UUAAAGAAAAUAUCAUUUGG | SEQ ID NO: 84 |
| | F508del* | | |
| TS34 | CFTR- | UAAAGAAAAUAUCAUAUUGG | SEQ ID NO: 85 |
| | F508del* | | |
| TS35 | CFTR- | UAAAGAAAAUAUCAUUUUGG | SEQ ID NO: 86 |
| | F508del* | | |
| TS36 | CFTR- | CAUCAUAGGAAACACCAAAA | SEQ ID NO: 87 |
| | F508del* | | |
| Lb1 | LMNA | UCUCCAAAUCCUGCAGGCGG | SEQ ID NO: 88 |
| | | GUC | |
| ROS1 | ROS1 | UUAAAUUUAGUUGAAGCAC | SEQ ID NO: 89 |
| sgRNA | | | |
| CD74 | CD74 | UCCUGAAGUAGAAGGUCAA | SEQ ID NO: 90 |
| sgRNA | | | |
| PDCD1 | PDCD1 | GGCGCCCUGGCCAGUCGUCU | SEQ ID NO: 91 |
| sgRNA | | | |
| TRBC1/2 | TRBC1/2 | GGAGAAUGACGAGUGGACCC | SEQ ID NO: 92 |
| sgRNA | | | |
| TRAC | TRAC | UGUGCUAGACAUGAGGUCUA | SEQ ID NO: 93 |
| sgRNA | | | |
| CFTR-g1 | CFTR-WT | AUUAAAGAAAAUAUCAUCUU | SEQ ID NO: 94 |
| DMD- | DMD | UAAGGGAUAUUUGUUCUUAC | SEQ ID NO: 95 |
| Ex52-g1 | | | |
| DMD- | DMD | AGAGGCUAGAACAAUCAUUA | SEQ ID NO: 96 |
| Ex52-g2 | | | |

*Intermediate products created during sequential CasPlus editing.

TABLE 2
Large deletions generated by Cas9 and CasPlus editing using guide RNA TS10 or
TS9 in male DMD-del52 cells.

| | No. of reads | | | | | | | |
|---|---|---|---|---|---|---|---|---|
| | TS10 | | | | TS9 | | | |
| Deletion | | | CasPlus- | CasPlus- | | | CasPlus- | CasPlus- |
| size (bp) | Untreated | Cas9 | V1 | V2 | Untreated | Cas9 | V1 | V2 |
| 201-500 | 0 | 19 | 0 | 0 | 0 | 11 | 0 | 2 |
| 501-1000 | 0 | 47 | 4 | 1 | 0 | 5 | 0 | 2 |
| 1001-1500 | 0 | 68 | 4 | 0 | 0 | 22 | 0 | 3 |
| 1501-2000 | 0 | 196 | 0 | 1 | 1 | 6 | 0 | 1 |
| 2001-2500 | 2 | 0 | 0 | 0 | 2 | 49 | 0 | 1 |
| 2501-3000 | 49 | 66 | 41 | 61 | 394 | 197 | 190 | 205 |
| 3001-3500 | 2 | 2 | 1 | 3 | 1 | 568 | 0 | 0 |
| 3501-4000 | 2 | 0 | 3 | 8 | 4 | 0 | 0 | 5 |
| 4001-4500 | 1 | 1 | 1 | 15 | 5 | 5 | 0 | 4 |
| 4501-5000 | 3 | 2 | 1 | 5 | 8 | 1 | 1 | 11 |
| 5001-5500 | NA | NA | NA | NA | 6 | 0 | 1 | 7 |
| Total* | 2902 | 1742 | 1699 | 2700 | 2988 | 1767 | 2029 | 1385 |

*Only those circular consensus sequencing (CCS) reads containing both the 5′ and 3′ index sequences were analyzed.

TABLE 3
Summary of the synthetic sequences and vector
information used in this disclosure.
CFTR-F508del knock-in template
taatcaaaaagttttcacatagtttcttacCTCTTCTAGTTGGCATGCTTTGATGACGCTTCTG
TATCTATATTCATCATAGGAAACACCAATGATATTTTCTTTAATGGTGCCAGGCATAATCCAG
(SEQ ID NO: 97).
tdTomato-d151A
atggtgagcaagggcgaggaggtcatcaaagagttcatgcgcttcaaggtgcgcatggagggct
ccatgaacggccacgagttcgagatcgagggcgagggcgagggccgcccctacgagggcaccca
gaccgccaagctgaaggtgaccagggcggccccctgcccttcgcctgggacatcctgtcccccc
agttcatgtacggctccaaggcgtacgtgaagcaccccgccgacatccccgattacaagaagct
gtccttccccgagggcttcaagtgggagcgcgtgatgaacttcgaggacggcggtctggtgacc
gtgacccaggactcctccctgcaggacggcacgctgatctacaaggtgaagatgcgcggcacca
acttcccccccgacggccccgtaatgcagaagaagaccatgggctgggaggcctccaccgagcg
cctgtacccccgcgacggcgtgctgaagggcgagatccaccaggccctgaagctgaaggacggc
ggccactacctggtggagttcaagaccatctacatggccaagaagcccgtgcaactgcccggct
actactacgtggacaccaagctggacatcacctcccacaacgaggactacaccatcgtggaaca
gtacgagcgctccgagggccgccaccacctgttcctggggcatggcaccggcagcaccggcagc
ggcagctccggcaccgcctcctccgaggacaacaacatggccgtcatcaaagagttcatgcgct
tcaaggtgcgcatggagggctccatgaacggccacgagttcgagatcgagggogagggcgaggg
ccgcccctacgagggcacccagaccgccaagctgaaggtgaccaagggcggccccctgcccttc
gcctgggacatcctgtccccccagttcatgtacggctccaaggcgtacgtgaagcaccccgccg
acatccccgattacaagaagctgtccttccccgagggcttcaagtgggagcgcgtgatgaactt
cgaggacggcggtctggtgaccgtgacccaggactcctccctgcaggacggcacgctgatctac
aaggtgaagatgcgcggcaccaacttcccccccgacggccccgtaatgcagaagaagaccatgg
gctgggaggcctccaccgagcgcctgtacccccgcgacggcgtgctgaagggcgagatccacca
ggccctgaagctgaaggacggcggccactacctggtggagttcaagaccatctacatggccaag
aagcccgtgcaactgcccggctactactacgtggacaccaagctggacatcacctcccacaacg
aggactacaccatcgtggaacagtacgagcgctccgagggccgccaccacctgttcctg (SEQ
ID NO: 98).
T4-D219A Protein sequence
MS2-Linker-NLS-T4-D219A-NLS
MASNFTQFVLVDNGGTGDVTVAPSNFANGVAEWISSNSRSQAYKVTCSVRQSSAQKR
KYTIKVEVPKVATQTVGGVELPVAAWRSYLNMELTIPIFATNSDCELIVKAMQGLLK
DGNPIPSAIAANSGIYSAGGGGSGGGGSGGGGSGPKKKRKV

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

PKKKRKVAAA (SEQ ID NO: 51).
T4-D219A DNA sequences
MS2-Linker-NLS-T4-D219A-NLS
atggcttcaaactttactcagttcgtgctcgtggacaatggtgggacaggggatgtgacagtgg
ctccttctaatttcgctaatggggtggcagagtggatcagctccaactcacggagccaggccta
caaggtgacatgcagcgtcaggcagtctagtgcccagaagagaaagtataccatcaaggtggag
gtccccaaagtggctacccagacagtgggcggagtcgaactgcctgtcgccgcttggaggtcct
acctgaacatggagctcactatcccaattttcgctaccaattctgactgtgaactcatcgtgaa
ggcaatgcaggggctcctcaaagacggtaatcctatcccttccgccatcgccgctaactcaggt
atctacagcgctggaggaggtggaagcggaggaggaggaagcggaggaggaggtagcggaccta
agaaaaagaggaaggtg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cctaagaaaaagaggaaggtg (SEQ ID NO: 52).
RB69 DNA polymerase protein sequences
MS2-Linker-NLS-T4-D219A-NLS
MASNFTQFVLVDNGGTGDVTVAPSNFANGVAEWISSNSRSQAYKVTCSVRQSSAQKR
KYTIKVEVPKVATQTVGGVELPVAAWRSYLNMELTIPIFATNSDCELIVKAMQGLLK
DGNPIPSAIAANSGIYSAGGGGSGGGGSGGGGSGPKKKRKV

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

PKKKRKVAAA (SEQ ID NO: 53).
RB69 DNA polymerase DNA sequences
MS2-Linker-NLS-RB69-NLS
atggcttcaaactttactcagttcgtgctcgtggacaatggtgggacaggggatgtgacagtgg
ctccttctaatttcgctaatggggtggcagagtggatcagctccaactcacggagccaggccta
caaggtgacatgcagcgtcaggcagtctagtgcccagaagagaaagtataccatcaaggtggag
gtccccaaagtggctacccagacagtgggcggagtcgaactgcctgtcgccgcttggaggtcct
acctgaacatggagctcactatcccaattttcgctaccaattctgactgtgaactcatcgtgaa
ggcaatgcaggggctcctcaaagacggtaatcctatcccttccgccatcgccgctaactcaggt
atctacagcgctggaggaggtggaagcggaggaggaggaagcggaggaggaggtagcggaccta
agaaaaagaggaaggtg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cctaagaaaaagaggaaggtg (SEQ ID NO: 54).
RB69-D222A Protein sequences
MS2-Linker-NLS-RB69-D222A-NLS
MASNFTQFVLVDNGGTGDVTVAPSNFANGVAEWISSNSRSQAYKVTCSVRQSSAQKR
KYTIKVEVPKVATQTVGGVELPVAAWRSYLNMELTIPIFATNSDCELIVKAMQGLLK
DGNPIPSAIAANSGIYSAGGGGSGGGGSGGGGSGPKKKRKV

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

PKKKRKVAAA (SEQ ID NO: 55).
RB69-D222A DNA sequences
MS2-Linker-NLS-RB69-D222A-NLS
atggcttcaaactttactcagttcgtgctcgtggacaatggtgggacaggggatgtgacagtgg
ctccttctaatttcgctaatggggtggcagagtggatcagctccaactcacggagccaggccta
caaggtgacatgcagcgtcaggcagtctagtgcccagaagagaaagtataccatcaaggtggag
gtccccaaagtggctacccagacagtgggcggagtcgaactgcctgtcgccgcttggaggtcct
acctgaacatggagctcactatcccaattttcgctaccaattctgactgtgaactcatcgtgaa
ggcaatgcaggggctcctcaaagacggtaatcctatcccttccgccatcgccgctaactcaggt
atctacagcgctggaggaggtggaagcggaggaggaggaagcggaggaggaggtagcggaccta
agaaaaagaggaaggtg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cctaagaaaaagaggaaggtg (SEQ ID NO: 56).
T7 DNA polymerase Protein sequence
MS2-Linker-NLS-T7-DNA-Pol-NLS
MASNFTQFVLVDNGGTGDVTVAPSNFANGVAEWISSNSRSQAYKVTCSVRQSSAQKR
KYTIKVEVPKVATQTVGGVELPVAAWRSYLNMELTIPIFATNSDCELIVKAMQGLLK
DGNPIPSAIAANSGIYSAGGGGSGGGGSGGGGSGPKKKRKV

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

PKKKRKVAAA (SEQ ID NO: 57).
T7 DNA polymerase DNA sequence
MS2-Linker-NLS-T7-DNA-Pol-NLS
atggcttcaaactttactcagttcgtgctcgtggacaatggtgggacaggggatgtgacagtgg
ctccttctaatttcgctaatggggtggcagagtggatcagctccaactcacggagccaggccta
caaggtgacatgcagcgtcaggcagtctagtgcccagaagagaaagtataccatcaaggtggag
gtccccaaagtggctacccagacagtgggcggagtcgaactgcctgtcgccgcttggaggtcct
acctgaacatggagctcactatcccaattttcgctaccaattctgactgtgaactcatcgtgaa
ggcaatgcaggggctcctcaaagacggtaatcctatcccttccgccatcgccgctaactcaggt
atctacagcgctggaggaggtggaagcggaggaggaggaagcggaggaggaggtagcggaccta
agaaaaagaggaaggtg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cctaagaaaaagaggaaggtg (SEQ
ID NO: 58).

What is claimed is:

1. A DNA polymerase protein that is optionally present in a fusion protein that comprises a segment of an MS2 bacteriophage coat protein, wherein the DNA polymerase is selected from:
i) T4 DNA polymerase, said T4 DNA polymerase comprising a mutation of D219, wherein the mutation is optionally a D219A mutation; and
ii) RB69 DNA polymerase, said RB69 comprising a mutation of D222, and wherein the mutation is optionally D222A.

2. The DNA polymerase protein of claim 1, wherein the DNA polymerase is the T4 DNA polymerase and comprises the D219A mutation.

3. The DNA polymerase of claim 1, wherein the DNA polymerase is the RB69 DNA polymerase protein and comprises the mutation of D222A.

4. The DNA polymerase of any one of claims 1-3, wherein the DNA polymerase protein is present in the fusion protein that comprises the segment of the MS2 bacteriophage coat protein.

5. A system for editing a DNA substrate, said system comprising the DNA polymerase protein of claim 4, and a Cas9 nuclease, said Cas9 nuclease optionally comprising a mutation selected from a mutation at position F916, R919 or Q920, wherein said mutations are optionally selected from F916P, F916del, R919P and Q920P, and a combination thereof.

6. The system of claim 5, wherein DNA polymerase is the T4 DNA polymerase protein and comprises a mutation of D219, and wherein the Cas9 nuclease comprises a mutation selected from F916P, F916del, R920P and Q920P.

7. The system of claim 6, further comprising at least one guide RNA that directs the system to a specific genomic location and creates an indel without using a DNA repair template, and wherein the guide RNA optionally comprises MS2 bacteriophage coat protein binding sites.

8. The system of claim 7, wherein the DNA polymerase protein comprises the segment of the MS2 bacteriophage coat protein.

9. The system of claim 5, wherein the DNA polymerase protein is the RB69 DNA polymerase protein that comprises the mutation of D222, and wherein the Cas9 nuclease comprises the mutation selected from F916P, F916del, R920P and Q920P.

10. The system of claim 9, further comprising at least one guide RNA that directs the system to a specific genomic location and creates an indel without using a DNA repair template, and wherein the guide RNA optionally comprises MS2 bacteriophage coat protein binding sites.

11. The system of claim 10, wherein the DNA polymerase protein comprises the segment of the MS2 bacteriophage coat protein.

12. A method comprising introducing the system of claim 5 into eukaryotic cells, wherein the DNA polymerase protein, the Cas9 nuclease, and an included guide RNA create an indel at a location in DNA that is determined by the sequence of the guide RNA.

13. The method of claim 12, wherein DNA polymerase is the T4 DNA polymerase protein and comprises a mutation of D219, and wherein the Cas9 nuclease that comprises a mutation selected from F916P, F916del, R920P and Q920P.

14. The method of claim 13, wherein the guide RNA optionally comprises MS2 bacteriophage coat protein binding sites.

15. The method of claim 13, wherein the DNA polymerase protein comprises the segment of the MS2 bacteriophage coat protein.

16. The method of claim 12, wherein the DNA polymerase protein is the RB69 DNA polymerase protein and comprises the mutation of D222, and wherein the Cas9 nuclease comprises the mutation selected from F916P, F916del, R920P and Q920P.

17. The method of claim 16, wherein the guide RNA optionally comprises MS2 bacteriophage coat protein binding sites.

18. The system of claim 17, wherein the DNA polymerase protein comprises the segment of the MS2 bacteriophage coat protein.

19. The method of claim 12, wherein the indel corrects a mutation in a gene associated with muscular dystrophy or cystic fibrosis.

20. The method of claim 12, wherein the eukaryotic cells are leukocytes.

21. The method of claim 20, wherein the eukaryotic cells leukocytes are T cells.

22. The method of claim 21, wherein the indel is in one or more of PDCD1, TRBC1, TRBC2, or TRAC.

23. The method of claim 22, wherein the T cells are also modified such that they express a chimeric antigen receptor.


SEQUENCE LISTING (98 sequences)

SEQ ID NO: 1 - DNA, 26 bp, synthetic construct
  source 1..26: mol_type = other DNA
ggtgggtgacctgggaattgattatt

SEQ ID NO: 2 - DNA, 26 bp, synthetic construct
  source 1..26: mol_type = other DNA
tattttaatatttgtcagtgggatga

SEQ ID NO: 3 - DNA, 46 bp, synthetic construct
  source 1..46: mol_type = other DNA
tgaccttgaggatatcaacgagatgatcatcaagcagaaggtatga

SEQ ID NO: 4 - DNA, 46 bp, synthetic construct
  source 1..46: mol_type = other DNA
tcataccttctgcttgatgatcatctcgttgatatcctcaaggtca

SEQ ID NO: 5 - DNA, 68 bp, synthetic construct
  source 1..68: mol_type = other DNA
agttgaaagaattcagaatcagtgggatgaagtacaagaacaccttcagaaccggaggcaacagttga

SEQ ID NO: 6 - DNA, 68 bp, synthetic construct
  source 1..68: mol_type = other DNA
tcaactgttgcctccggttctgaaggtgttcttgtacttcatcccactgattctgaattctttcaact

SEQ ID NO: 7 - DNA, 28 bp, synthetic construct
  source 1..28: mol_type = other DNA
cactattggagcctttgaaagaattcag

SEQ ID NO: 8 - DNA, 28 bp, synthetic construct
  source 1..28: mol_type = other DNA
tcatcaagcagaagcagttggccaaaga

SEQ ID NO: 9 - DNA, 28 bp, synthetic construct
  source 1..28: mol_type = other DNA
gcaccattaaagaaaatatcatctttgg

SEQ ID NO: 10 - DNA, 25 bp, synthetic construct
  source 1..25: mol_type = other DNA
gcaccattaaagaaaatatcattgg

SEQ ID NO: 11 - DNA, 28 bp, synthetic construct
  source 1..28: mol_type = other DNA
gcaccattaaagaaaatatcatctttgg

SEQ ID NO: 12 - DNA, 25 bp, synthetic construct
  source 1..25: mol_type = other DNA
ccaatgatattttctttaatggtgc

SEQ ID NO: 13 - DNA, 18 bp, synthetic construct
  source 1..18: mol_type = other DNA
aatatcatctttggtgtt

SEQ ID NO: 14 - DNA, 18 bp, synthetic construct
  source 1..18: mol_type = other DNA
aatatcatcattggtgtt

SEQ ID NO: 15 - DNA, 18 bp, synthetic construct
  source 1..18: mol_type = other DNA
aatatcatatttggtgtt

SEQ ID NO: 16 - DNA, 18 bp, synthetic construct
  source 1..18: mol_type = other DNA
aatatcatttttggtgtt

SEQ ID NO: 17 - DNA, 35 bp, synthetic construct
  source 1..35: mol_type = other DNA
attaaagaaaatatcattggtgtttcctatgatga

SEQ ID NO: 18 - DNA, 35 bp, synthetic construct
  source 1..35: mol_type = other DNA
tcatcataggaaacaccaatgatattttctttaat

SEQ ID NO: 19 - DNA, 36 bp, synthetic construct
  source 1..36: mol_type = other DNA
attaaagaaaatatcatttggtgtttcctatgatga

SEQ ID NO: 20 - DNA, 36 bp, synthetic construct
  source 1..36: mol_type = other DNA
tcatcataggaaacaccaaatgatattttctttaat

SEQ ID NO: 21 - DNA, 37 bp, synthetic construct
  source 1..37: mol_type = other DNA
attaaagaaaatatcatattggtgtttcctatgatga

SEQ ID NO: 22 - DNA, 37 bp, synthetic construct
  source 1..37: mol_type = other DNA
tcatcataggaaacaccaatatgatattttctttaat

SEQ ID NO: 23 - DNA, 37 bp, synthetic construct
  source 1..37: mol_type = other DNA
attaaagaaaatatcattttggtgtttcctatgatga

SEQ ID NO: 24 - DNA, 37 bp, synthetic construct
  source 1..37: mol_type = other DNA
tcatcataggaaacaccaaaatgatattttctttaat

SEQ ID NO: 25 - DNA, 11 bp, synthetic construct
  source 1..11: mol_type = other DNA
gaagcaaaggg

SEQ ID NO: 26 - DNA, 12 bp, synthetic construct
  source 1..12: mol_type = other DNA
gaagtacaggct

SEQ ID NO: 27 - DNA, 12 bp, synthetic construct
  source 1..12: mol_type = other DNA
cccagacccagg

SEQ ID NO: 28 - DNA, 12 bp, synthetic construct
  source 1..12: mol_type = other DNA
agcccacccagg

SEQ ID NO: 29 - DNA, 12 bp, synthetic construct
  source 1..12: mol_type = other DNA
cccagatctatg

SEQ ID NO: 30 - DNA, 12 bp, synthetic construct
  source 1..12: mol_type = other DNA
agtggacgactg

SEQ ID NO: 31 - DNA, 12 bp, synthetic construct
  source 1..12: mol_type = other DNA
agtggatctatg

SEQ ID NO: 32 - DNA, 12 bp, synthetic construct
  source 1..12: mol_type = other DNA
tgaggtcccagg

SEQ ID NO: 33 - DNA, 12 bp, synthetic construct
  source 1..12: mol_type = other DNA
tgaggtcccagg

SEQ ID NO: 34 - DNA, 12 bp, synthetic construct
  source 1..12: mol_type = other DNA
gcgcccaggata

SEQ ID NO: 35 - DNA, 12 bp, synthetic construct
  source 1..12: mol_type = other DNA
ccagtccccagg

SEQ ID NO: 36 - DNA, 12 bp, synthetic construct
  source 1..12: mol_type = other DNA
ccagtctatgga

SEQ ID NO: 37 - DNA, 12 bp, synthetic construct
  source 1..12: mol_type = other DNA
agtggatctggg

SEQ ID NO: 38 - DNA, 12 bp, synthetic construct
  source 1..12: mol_type = other DNA
tgaggttctggg

SEQ ID NO: 39 - DNA, 12 bp, synthetic construct
  source 1..12: mol_type = other DNA
cctggggacttc

SEQ ID NO: 40 - DNA, 12 bp, synthetic construct
  source 1..12: mol_type = other DNA
cctgggctatgg

SEQ ID NO: 41 - DNA, 12 bp, synthetic construct
  source 1..12: mol_type = other DNA
agtggaacctca

SEQ ID NO: 42 - protein, 18 aa, synthetic construct
EGRGSLLTCGDVEENPGP

SEQ ID NO: 43 - protein, 19 aa, synthetic construct
ATNFSLLKQAGDVEENPGP

SEQ ID NO: 44 - protein, 20 aa, synthetic construct
QCTNYALLKLAGDVESNPGP

SEQ ID NO: 45 - protein, 22 aa, synthetic construct
VKQTLNFDLLKLAGDVESNPGP

SEQ ID NO: 46 - protein, 18 aa, synthetic construct
SAGGGGSGGGGSGGGGSG

SEQ ID NO: 47 - protein, 897 aa, synthetic construct
KEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 48 - protein, 130 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 49 - protein, 11 aa, synthetic construct
GPKKKRKVAAA

SEQ ID NO: 50 - protein, 1065 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFGGSGPKKKRKVAAA

SEQ ID NO: 51 - protein, 1065 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFAVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFGGSGPKKKRKVAAA

SEQ ID NO: 52 - DNA, 3186 bp, synthetic construct
  source 1..3186: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtgaaggaattctacatcagcatcgagaccgtgggtaacaacatcgtggaaagatatattgacgaaaacggcaaggagagaaccagagaggtggaatacctgcctacaatgttccggcactgtaaagaggaatccaagtacaaggatatctacggcaaaaactgcgcccctcagaaattccccagcatgaaagacgccagagattggatgaagagaatggaggatatcggactggaagccctgggcatgaacgatttcaagctggcctacatctccgatacatacggaagcgagatcgtgtatgatagaaaattcgtgcgggtggccaattgtgacattgaggtgaccggcgacaagttccctgatcccatgaaagctgaatatgagatcgacgccattacccactacgacagcatcgacgacagattctacgtgttcgacctgctgaactccatgtacggcagcgtgtccaagtgggacgctaagctggccgccaagctggactgcgagggcggcgacgaggttccacaagagatcctggaccgggtcatctacatgcccttcgacaacgagagggacatgctgatggaatacatcaacctgtgggagcagaagcgccccgccatttttacaggctggaacatcgagggcttcgccgtgccttatatcatgaatagagtgaaaatgatcctgggagaacggagcatgaaaagattcagccctatcggcagagtgaagagcaagctgatccaaaacatgtacggctccaaggaaatctatagcatcgatggcgtgtccatcctggattacctggacctgtacaaaaagttcgccttcaccaacctgccatctttctctcttgagagcgtcgcccagcacgagacaaagaagggcaagctgccgtacgacggtcctatcaacaagctgagagaaacaaatcaccagagatacatcagctacaacatcatcgatgtggaaagcgttcaggccatcgataaaatcagaggcttcatcgacctggtgctgtctatgtcttactacgccaagatgccttttagcggagtgatgagccctatcaagacctgggatgccatcatcttcaacagcctgaagggcgaacacaaggtgatcccccaacagggcagccacgtgaagcagagcttcccaggcgcttttgtgttcgagcccaagcccatagcgcggagatacatcatgagctttgatctgaccagcctgtaccccagcatcattcggcaagtgaacatttctccagaaaccatcagaggccagtttaaggtgcaccctatccacgagtatattgcaggcaccgctcctaaacctagcgacgagtacagctgctctcctaacggctggatgtacgacaagcaccaggagggaatcatccctaaggaaattgccaaggtgtttttccagcggaaggactggaagaaaaaaatgttcgccgaggaaatgaacgccgaggccatcaagaagatcatcatgaagggcgccggcagctgctccaccaagcctgaggtggaaagatacgtgaagttcagcgacgatttcctgaatgagctcagcaactacaccgagtctgtcctgaactcactgattgaggaatgcgagaaggccgccaccctggctaataccaaccagctgaaccggaagattctgatcaacagcctgtacggagctctgggcaatattcacttcagatactacgatctgcgaaacgccacagctattacaattttcggccaggtgggcatccagtggatcgccagaaagatcaatgagtacctgaacaaggtgtgcggcaccaacgacgaggacttcatcgccgctggcgatactgatagcgtgtacgtttgtgtggacaaggtcatcgagaaggttggcctggacagatttaaggaacagaacgacctcgtggagttcatgaaccagttcggaaagaagaagatggaacccatgatcgatgtggcttatagagagctgtgcgactacatgaacaacagagagcacctgatgcacatggatagagaagctatttcttgccctcctctgggctctaagggagtgggcggattttggaaagccaaaaagagatacgccctgaatgtgtacgacatggaagataagagattcgccgagcctcacctgaaaatcatgggcatggaaacacagcagagcagcacccctaaggctgtgcaggaggccctggaagagtctatccggagaatcttgcaggagggcgaggaaagcgtgcaggagtactacaagaacttcgagaaagaatacagacagctggactacaaggtgatcgcggaggtgaagaccgctaatgatatcgccaagtacgacgacaagggctggcccggcttcaagtgccccttccacatcagaggcgtgctcacctaccgcagagccgtttccggcctgggcgtggcccctatcctggatggaaacaaagtcatggtgctgcctctgagagagggcaacccctttggagataaatgcatcgcttggcctagcggcactgagctgcccaaggaaatccgctccgacgtgctgagctggatcgatcacagcaccctgttccaaaagtccttcgtgaagcccctggccggcatgtgcgagtccgccggcatggactacgaggaaaaggccagcctggatttcctgttcggcggatccggacctaagaaaaagaggaaggtg

SEQ ID NO: 53 - protein, 1070 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVKEFYLTVEQIGDSIFERYIDSNGRERTREVEYKPSLFAHCPESQATKYFDIYGKPCTRKLFANMRDASQWIKRMEDIGLEALGMDDFKLAYLSDTYNYEIKYDHTKIRVANFDIEVTSPDGFPEPSQAKHPIDAITHYDSIDDRFYVFDLLNSPYGNVEEWSIEIAAKLQEQGGDEVPSEIIDKIIYMPFDNEKELLMEYLNFWQQKTPVILTGWNVESFDIPYVYNRIKNIFGESTAKRLSPHRKTRVKVIENMYGSREIITLFGISVLDYIDLYKKFSFTNQPSYSLDYISEFELNVGKLKYDGPISKLRESNHQRYISYNIIDVYRVLQIDAKRQFINLSLDMGYYAKIQIQSVFSPIKTWDAIIFNSLKEQNKVIPQGRSHPVQPYPGAFVKEPIPNRYKYVMSFDLTSLYPSIIRQVNISPETIAGTFKVAPLHDYINAVAERPSDVYSCSPNGMMYYKDRDGVVPTEITKVFNQRKEHKGYMLAAQRNGEIIKEALHNPNLSVDEPLDVDYRFDFSDEIKEKIKKLSAKSLNEMLFRAQRTEVAGMTAQINRKLLINSLYGALGNVWFRYYDLRNATAITTFGQMALQWIERKVNEYLNEVCGTEGEAFVLYGDTDSIYVSADKIIDKVGESKFRDTNHWVDFLDKFARERMEPAIDRGFREMCEYMNNKQHLMFMDREAIAGPPLGSKGIGGFWTGKKRYALNVWDMEGTRYAEPKLKIMGLETQKSSTPKAVQKALKECIRRMLQEGEESLQEYFKEFEKEFRQLNYISIASVSSANNIAKYDVGGFPGPKCPFHIRGILTYNRAIKGNIDAPQVVEGEKVYVLPLREGNPFGDKCIAWPSGTEITDLIKDDVLHWMDYTVLLEKTFIKPLEGFTSAAKLDYEKKASLFDMFDFGSGPKKKRKVAAA

SEQ ID NO: 54 - DNA, 3201 bp, synthetic construct
  source 1..3201: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtgaaggagttctacctgaccgtggagcagatcggcgacagcatcttcgagagatacatcgacagcaacggcagagagagaacaagagaggtggagtacaagccaagcctgtttgcgcactgccccgagagccaagccaccaagtacttcgacatctacggcaagccctgcacaagaaagctgttcgccaacatgagagacgcttctcagtggatcaagagaatggaggacatcggcctggaggccctgggcatggacgacttcaagctggcctacctgagcgacacctacaactacgagatcaagtacgaccacaccaagatcagagtggccaacttcgacatcgaggtgacaagccccgacggcttccccgagcctagccaagccaagcaccccatcgacgccatcacccactacgacagcatcgacgacagattctacgtgttcgacctgctgaacagcccctacggcaacgtggaggagtggagcatcgagatcgccgcgaagctgcaagagcaaggcggcgacgaggtgcctagcgaaatcatcgataagataatctacatgcccttcgacaacgagaaggagctgctgatggaatatctgaacttttggcagcagaagacccccgtgatcctgaccggctggaacgtggagagcttcgacatcccctacgtgtacaacagaatcaagaacatcttcggcgagagcaccgccaagagactgagcccccacagaaagacaagagtgaaggtgatcgagaacatgtacggcagcagagagatcatcaccctgttcggcatcagcgtgctggactacatagacctgtacaagaagttcagcttcaccaatcagcctagctacagcctggactacatcagtgaattcgagctgaacgtgggcaagctgaagtacgacggccccatcagcaagctgagagagagcaaccatcagagatacatcagctacaacatcatcgatgtgtaccgcgtactgcagatcgacgccaagagacagttcatcaacctgagcctggacatgggctactacgccaagattcagattcagagcgtgttcagccccatcaagacctgggacgccatcatcttcaacagcctgaaggagcagaacaaggtgatcccccaaggcagaagccaccccgtgcagccctaccccggcgccttcgtgaaggagcccatccccaacagatacaagtacgtgatgagcttcgacctgacaagcctgtaccctagcatcatcagacaagtgaacatcagccccgagaccatcgccggcaccttcaaggtggcccccctgcacgactatatcaatgccgtggccgagagacctagcgatgtctacagctgcagccccaacggcatgatgtactacaaggacagagacggcgtggtgcccaccgaaatcacgaaagtgttcaatcagagaaaggagcacaagggctacatgctggccgctcagagaaacggcgagatcatcaaggaggccctgcacaaccccaacctgagcgtggacgagcccctggacgtggactacagattcgacttcagcgacgagatcaaggagaagatcaagaagctgagcgccaagagcctgaacgagatgctgttcagagctcagagaaccgaggtggccggcatgaccgctcagatcaacagaaagctgctgatcaacagcctgtacggcgccctgggcaacgtgtggttcagatactacgacctgagaaacgccaccgccatcaccaccttcgggcagatggccctgcagtggatcgagagaaaggtgaacgaatacctgaacgaggtatgcggcaccgagggcgaggccttcgtgctgtacggcgacaccgacagcatctacgtgagcgccgacaagataatagacaaggtgggtgagagcaagttcagagacaccaaccactgggtggacttcctggacaagttcgctagagagagaatggagcccgccatcgacagaggcttcagagagatgtgcgagtacatgaacaacaagcagcacctgatgttcatggacagagaggccatcgccggcccccccctgggcagcaagggcatcggcggcttctggaccggcaagaagagatacgccctgaacgtgtgggacatggagggcacaagatacgccgagcccaagctgaagatcatgggcctggagacacagaagagcagcacccccaaggccgtgcagaaggccctgaaggagtgcatcagaagaatgctgcaagagggcgaggagagcctgcaagagtacttcaaggagttcgagaaggagttcagacagctgaattacatcagcatcgcaagcgtgagcagcgccaacaacatcgccaagtacgacgtgggcggcttccccggccccaagtgccccttccacatcagaggcatcctgacctacaacagagccatcaagggcaacatcgacgccccccaagtggtggagggcgagaaggtgtacgtgctgcccctgagagagggcaaccccttcggcgacaagtgcatcgcctggcctagcggcaccgagatcaccgacctgatcaaggacgacgtgctgcactggatggactacaccgtgctgctggagaagaccttcatcaagcccctggagggcttcacaagcgccgccaagctggattacgagaagaaggctagcctctttgacatgttcgacttcggatccggacctaagaaaaagaggaaggtg

SEQ ID NO: 55 - protein, 1070 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVKEFYLTVEQIGDSIFERYIDSNGRERTREVEYKPSLFAHCPESQATKYFDIYGKPCTRKLFANMRDASQWIKRMEDIGLEALGMDDFKLAYLSDTYNYEIKYDHTKIRVANFDIEVTSPDGFPEPSQAKHPIDAITHYDSIDDRFYVFDLLNSPYGNVEEWSIEIAAKLQEQGGDEVPSEIIDKIIYMPFDNEKELLMEYLNFWQQKTPVILTGWNVESFAIPYVYNRIKNIFGESTAKRLSPHRKTRVKVIENMYGSREIITLFGISVLDYIDLYKKFSFTNQPSYSLDYISEFELNVGKLKYDGPISKLRESNHQRYISYNIIDVYRVLQIDAKRQFINLSLDMGYYAKIQIQSVFSPIKTWDAIIFNSLKEQNKVIPQGRSHPVQPYPGAFVKEPIPNRYKYVMSFDLTSLYPSIIRQVNISPETIAGTFKVAPLHDYINAVAERPSDVYSCSPNGMMYYKDRDGVVPTEITKVFNQRKEHKGYMLAAQRNGEIIKEALHNPNLSVDEPLDVDYRFDFSDEIKEKIKKLSAKSLNEMLFRAQRTEVAGMTAQINRKLLINSLYGALGNVWFRYYDLRNATAITTFGQMALQWIERKVNEYLNEVCGTEGEAFVLYGDTDSIYVSADKIIDKVGESKFRDTNHWVDFLDKFARERMEPAIDRGFREMCEYMNNKQHLMFMDREAIAGPPLGSKGIGGFWTGKKRYALNVWDMEGTRYAEPKLKIMGLETQKSSTPKAVQKALKECIRRMLQEGEESLQEYFKEFEKEFRQLNYISIASVSSANNIAKYDVGGFPGPKCPFHIRGILTYNRAIKGNIDAPQVVEGEKVYVLPLREGNPFGDKCIAWPSGTEITDLIKDDVLHWMDYTVLLEKTFIKPLEGFTSAAKLDYEKKASLFDMFDFGSGPKKKRKVAAA

SEQ ID NO: 56 - DNA, 3201 bp, synthetic construct
  source 1..3201: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtgaaggagttctacctgaccgtggagcagatcggcgacagcatcttcgagagatacatcgacagcaacggcagagagagaacaagagaggtggagtacaagccaagcctgtttgcgcactgccccgagagccaagccaccaagtacttcgacatctacggcaagccctgcacaagaaagctgttcgccaacatgagagacgcttctcagtggatcaagagaatggaggacatcggcctggaggccctgggcatggacgacttcaagctggcctacctgagcgacacctacaactacgagatcaagtacgaccacaccaagatcagagtggccaacttcgacatcgaggtgacaagccccgacggcttccccgagcctagccaagccaagcaccccatcgacgccatcacccactacgacagcatcgacgacagattctacgtgttcgacctgctgaacagcccctacggcaacgtggaggagtggagcatcgagatcgccgcgaagctgcaagagcaaggcggcgacgaggtgcctagcgaaatcatcgataagataatctacatgcccttcgacaacgagaaggagctgctgatggaatatctgaacttttggcagcagaagacccccgtgatcctgaccggctggaacgtggagagcttcgccatcccctacgtgtacaacagaatcaagaacatcttcggcgagagcaccgccaagagactgagcccccacagaaagacaagagtgaaggtgatcgagaacatgtacggcagcagagagatcatcaccctgttcggcatcagcgtgctggactacatagacctgtacaagaagttcagcttcaccaatcagcctagctacagcctggactacatcagtgaattcgagctgaacgtgggcaagctgaagtacgacggccccatcagcaagctgagagagagcaaccatcagagatacatcagctacaacatcatcgatgtgtaccgcgtactgcagatcgacgccaagagacagttcatcaacctgagcctggacatgggctactacgccaagattcagattcagagcgtgttcagccccatcaagacctgggacgccatcatcttcaacagcctgaaggagcagaacaaggtgatcccccaaggcagaagccaccccgtgcagccctaccccggcgccttcgtgaaggagcccatccccaacagatacaagtacgtgatgagcttcgacctgacaagcctgtaccctagcatcatcagacaagtgaacatcagccccgagaccatcgccggcaccttcaaggtggcccccctgcacgactatatcaatgccgtggccgagagacctagcgatgtctacagctgcagccccaacggcatgatgtactacaaggacagagacggcgtggtgcccaccgaaatcacgaaagtgttcaatcagagaaaggagcacaagggctacatgctggccgctcagagaaacggcgagatcatcaaggaggccctgcacaaccccaacctgagcgtggacgagcccctggacgtggactacagattcgacttcagcgacgagatcaaggagaagatcaagaagctgagcgccaagagcctgaacgagatgctgttcagagctcagagaaccgaggtggccggcatgaccgctcagatcaacagaaagctgctgatcaacagcctgtacggcgccctgggcaacgtgtggttcagatactacgacctgagaaacgccaccgccatcaccaccttcgggcagatggccctgcagtggatcgagagaaaggtgaacgaatacctgaacgaggtatgcggcaccgagggcgaggccttcgtgctgtacggcgacaccgacagcatctacgtgagcgccgacaagataatagacaaggtgggtgagagcaagttcagagacaccaaccactgggtggacttcctggacaagttcgctagagagagaatggagcccgccatcgacagaggcttcagagagatgtgcgagtacatgaacaacaagcagcacctgatgttcatggacagagaggccatcgccggcccccccctgggcagcaagggcatcggcggcttctggaccggcaagaagagatacgccctgaacgtgtgggacatggagggcacaagatacgccgagcccaagctgaagatcatgggcctggagacacagaagagcagcacccccaaggccgtgcagaaggccctgaaggagtgcatcagaagaatgctgcaagagggcgaggagagcctgcaagagtacttcaaggagttcgagaaggagttcagacagctgaattacatcagcatcgcaagcgtgagcagcgccaacaacatcgccaagtacgacgtgggcggcttccccggccccaagtgccccttccacatcagaggcatcctgacctacaacagagccatcaagggcaacatcgacgccccccaagtggtggagggcgagaaggtgtacgtgctgcccctgagagagggcaaccccttcggcgacaagtgcatcgcctggcctagcggcaccgagatcaccgacctgatcaaggacgacgtgctgcactggatggactacaccgtgctgctggagaagaccttcatcaagcccctggagggcttcacaagcgccgccaagctggattacgagaagaaggctagcctctttgacatgttcgacttcggatccggacctaagaaaaagaggaaggtg

SEQ ID NO: 57 - protein, 871 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSGPKKKRKVIVSDIEANALLESVTKFHCGVIYDYSTAEYVSYRPSDFGAYLDALEAEVARGGLIVFHNGHKYDVPALTKLAKLQLNREFHLPRENCIDTLVLSRLIHSNLKDTDMGLLRSGKLPGKRFGSHALEAWGYRLGEMKGEYKDDFKRMLEEQGEEYVDGMEWWNFNEEMMDYNVQDVVVTKALLEKLLSDKHYFPPEIDFTDVGYTTFWSESLEAVDIEHRAAWLLAKQERNGFPFDTKAIEELYVELAARRSELLRKLTETFGSWYQPKGGTEMFCHPRTGKPLPKYPRIKTPKVGGIFKKPKNKAQREGREPCELDTREYVAGAPYTPVEHVVFNPSSRDHIQKKLQEAGWVPTKYTDKGAPVVDDEVLEGVRVDDPEKQAAIDLIKEYLMIQKRIGQSAEGDKAWLRYVAEDGKIHGSVNPNGAVTGRATHAFPNLAQIPGVRSPYGEQCRAAFGAEHHLDGITGKPWVQAGIDASGLELRCLAHFMARFDNGEYAHEILNGDIHTKNQIAAELPTRDNAKTFIYGFLYGAGDEKIGQIVGAGKERGKELKKKFLENTPAIAALRESIQQTLVESSQWVAGEQQVKWKRRWIKGLDGRKVHVRSPHAALNTLLQSAGALICKLWIIKTEEMLVEKGLKHGWDGDFAYMAWVHDEIQVGCRTEEIAQVVIETAQEAMRWVGDHWNFRCLLDTEGKMGPNWAICHGSGPKKKRKVAAA

SEQ ID NO: 58 - DNA, 2604 bp, synthetic construct
  source 1..2604: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggaggaggtggaagcggaggaggaggaagcggaggaggaggtagcggacctaagaaaaagaggaaggtgatcgtgagcgacatcgaggccaacgccctgctggagagcgttaccaagttccactgcggcgtgatctacgactacagcaccgccgagtacgtgagctacagacctagcgacttcggcgcctacctggatgctctggaggccgaagtcgctagaggcggcctgatcgtgttccacaacggccacaagtacgacgtgcccgccctgaccaagctggccaagctgcagctgaacagagagttccacctgcctagagagaactgcatcgacaccctggtgctgagcagactgatccacagcaacctgaaggacaccgacatgggcctgctgagaagcggcaagctgcccggcaagagattcggcagccacgccctagaggcctggggttacagactgggcgagatgaagggcgagtacaaggacgacttcaagagaatgctggaggagcaaggcgaggagtacgtggacggcatggagtggtggaacttcaacgaggagatgatggactacaacgtgcaagacgtggtggtgaccaaggcactgctggagaaattgctgagcgacaagcactacttcccccccgagatcgacttcaccgacgtgggctacaccaccttctggagcgagagcctggaggccgtggacatcgagcacagagccgcctggctgctggccaagcaagagagaaacggcttccccttcgacaccaaggccatcgaggagctgtacgtggagctggccgctagaagaagcgagctgctgagaaagctgaccgagaccttcggcagctggtatcagcccaagggcggcaccgagatgttctgccaccctagaaccggaaagccgctccccaagtaccctagaatcaagacccccaaggtgggcggcatcttcaagaagcccaagaacaaggctcagagagagggcagagagccctgcgagctggacacaagagagtacgtggccggcgccccctacacccccgtggagcacgtggtgttcaaccctagcagcagagaccacattcagaagaagctgcaagaggccggctgggtgcccaccaagtacaccgacaagggcgcccccgtggttgacgacgaagtactggagggcgtgagagtggacgaccccgagaagcaagccgccatcgacctgatcaaggagtacctgatgattcagaagagaatcgggcagagcgccgagggcgacaaggcctggctgagatacgtggccgaggacggcaaaattcacggctcggtcaaccccaacggcgccgtaaccggcagagcaacccacgccttccccaacctggctcagatccccggcgtaagaagtccttatggggaacagtgccgcgcggcgtttggcgccgagcaccatctggatggcataaccggcaagccctgggtgcaagccggcatcgatgctagcggcctggagctgagatgcctggcccacttcatggctagattcgacaacggcgagtacgcccacgagatcctgaacggcgacatccacaccaagaatcagatcgccgccgagctgcccacaagagacaacgccaagaccttcatctacggcttcctgtacggcgccggcgacgagaagatcgggcagatcgtgggcgccggcaaggagagaggcaaggagctgaagaagaagttcctggagaacacccccgccatcgccgccctgagagagagcattcagcagaccctggtggagagctctcagtgggtggccggcgaacagcaagtcaagtggaagagaagatggatcaagggcctggacggcagaaaggtgcacgtgagaagtccgcacgccgccctgaacaccctgctgcagagcgccggcgccctgatctgcaagctgtggatcatcaagaccgaggagatgctggtggagaagggcctgaagcacggctgggacggcgacttcgcctacatggcctgggtgcacgacgagatccaagtgggctgcagaaccgaggagatcgcccaagtggtgatcgagaccgcccaagaggccatgagatgggtgggcgaccactggaacttcagatgcctgctggacaccgagggcaagatgggccccaactgggccatctgccacggatccggacctaagaaaaagaggaaggtg

SEQ ID NO: 59 - RNA, 163 nt, synthetic construct
  source 1..163: mol_type = other RNA
nnnnnnnnnnnnnnnnnnnngttttagagctaggccaacatgaggatcacccatgtctgcagggcctagcaagttaaaataaggctagtccgttatcaacttggccaacatgaggatcacccatgtctgcagggccaagtggcaccgagtcggtgcttttttt

SEQ ID NO: 60 - DNA, 50 bp, synthetic construct
  source 1..50: mol_type = other DNA
tttttccaaacgtgcttttcaggaaacagtggtctgcttgttgaagtctg

SEQ ID NO: 61 - DNA, 50 bp, synthetic construct
  source 1..50: mol_type = other DNA
aatcctggaccagaggttccattgagctgagatcacaccattgcactcca

SEQ ID NO: 62 - DNA, 50 bp, synthetic construct
  source 1..50: mol_type = other DNA
ggactatatttttgatttcatgttacaatcactagttttgtggggtcttt

SEQ ID NO: 63 - DNA, 50 bp, synthetic construct
  source 1..50: mol_type = other DNA
tgatgtgtattgctgcagattcaatgtaagttcccgatacagataaagat

SEQ ID NO: 64 - RNA, 20 nt, synthetic construct
  source 1..20: mol_type = other RNA
tccaggaacagctgggtacc

SEQ ID NO: 65 - RNA, 20 nt, synthetic construct
  source 1..20: mol_type = other RNA
attaaagaaaatatcatctt

SEQ ID NO: 66 - RNA, 20 nt, synthetic construct
  source 1..20: mol_type = other RNA
accttcactggctgagtggc

SEQ ID NO: 67 - RNA, 20 nt, synthetic construct
  source 1..20: mol_type = other RNA
ttgaaagaattcagaatcag

SEQ ID NO: 68 - RNA, 20 nt, synthetic construct
  source 1..20: mol_type = other RNA
tcatctcgttgatatcctca

SEQ ID NO: 69 - RNA, 20 nt, synthetic construct
  source 1..20: mol_type = other RNA
tcctactcagactgttactc

SEQ ID NO: 70 - RNA, 20 nt, synthetic construct
  source 1..20: mol_type = other RNA
ggggccaggtggccaaggtg

SEQ ID NO: 71 - RNA, 20 nt, synthetic construct
  source 1..20: mol_type = other RNA
tatgtgttacctacccttgt

SEQ ID NO: 72 - RNA, 20 nt, synthetic construct
  source 1..20: mol_type = other RNA
ggttgcttcattaccttcac

SEQ ID NO: 73 - RNA, 20 nt, synthetic construct
  source 1..20: mol_type = other RNA
tacctgaaccgtatatccta

SEQ ID NO: 74 - RNA, 20 nt, synthetic construct
  source 1..20: mol_type = other RNA
tccaggatggcattgggcag

SEQ ID NO: 75 - RNA, 20 nt, synthetic construct
  source 1..20: mol_type = other RNA
accagagtaacagtctgagt

SEQ ID NO: 76 - RNA, 20 nt, synthetic construct
  source 1..20: mol_type = other RNA
tataaaatcacagagggtga

SEQ ID NO: 77 - RNA, 20 nt, synthetic construct
  source 1..20: mol_type = other RNA
cctgcagggtggcctcacct

SEQ ID NO: 78 - RNA, 20 nt, synthetic construct
  source 1..20: mol_type = other RNA
cgagatgatcatcaagcaga

SEQ ID NO: 79 - RNA, 20 nt, synthetic construct
  source 1..20: mol_type = other RNA
tacaagaacaccttcagaac

SEQ ID NO: 80 - RNA, 20 nt, synthetic construct
  source 1..20: mol_type = other RNA
aagaacaccttcagaaccgg

SEQ ID NO: 81 - RNA, 20 nt, synthetic construct
  source 1..20: mol_type = other RNA
actgttgcctccggttctga

SEQ ID NO: 82 - RNA, 20 nt, synthetic construct
  source 1..20: mol_type = other RNA
tttcattcaactgttgcctc

SEQ ID NO: 83 - RNA, 20 nt, synthetic construct
  source 1..20: mol_type = other RNA
attaaagaaaatatcattgg

SEQ ID NO: 84 - RNA, 20 nt, synthetic construct
  source 1..20: mol_type = other RNA
ttaaagaaaatatcatttgg

SEQ ID NO: 85 - RNA, 20 nt, synthetic construct
  source 1..20: mol_type = other RNA
taaagaaaatatcatattgg

SEQ ID NO: 86 - RNA, 20 nt, synthetic construct
  source 1..20: mol_type = other RNA
taaagaaaatatcattttgg

SEQ ID NO: 87 - RNA, 20 nt, synthetic construct
  source 1..20: mol_type = other RNA
catcataggaaacaccaaaa

SEQ ID NO: 88 - RNA, 23 nt, synthetic construct
  source 1..23: mol_type = other RNA
tctccaaatcctgcaggcgggtc

SEQ ID NO: 89 - RNA, 19 nt, synthetic construct
  source 1..19: mol_type = other RNA
ttaaatttagttgaagcac

SEQ ID NO: 90 - RNA, 19 nt, synthetic construct
  source 1..19: mol_type = other RNA
tcctgaagtagaaggtcaa

SEQ ID NO: 91 - RNA, 20 nt, synthetic construct
  source 1..20: mol_type = other RNA
ggcgccctggccagtcgtct

SEQ ID NO: 92 - RNA, 20 nt, synthetic construct
  source 1..20: mol_type = other RNA
ggagaatgacgagtggaccc

SEQ ID NO: 93 - RNA, 20 nt, synthetic construct
  source 1..20: mol_type = other RNA
tgtgctagacatgaggtcta

SEQ ID NO: 94 - RNA, 20 nt, synthetic construct
  source 1..20: mol_type = other RNA
attaaagaaaatatcatctt

SEQ ID NO: 95 - RNA, 20 nt, synthetic construct
  source 1..20: mol_type = other RNA
taagggatatttgttcttac

SEQ ID NO: 96 - RNA, 20 nt, synthetic construct
  source 1..20: mol_type = other RNA
agaggctagaacaatcatta

SEQ ID NO: 97 - DNA, 127 bp, synthetic construct
  source 1..127: mol_type = other DNA
taatcaaaaagttttcacatagtttcttacctcttctagttggcatgctttgatgacgcttctgtatctatattcatcataggaaacaccaatgatattttctttaatggtgccaggcataatccag

SEQ ID NO: 98 - DNA, 1403 bp, synthetic construct
  source 1..1403: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctg